(19) United States
(12) Patent Application Publication
(10) Pub. No.: US 2012/0135424 A1
(43) Pub. Date: May 31, 2012

(54) MER DIAGNOSTIC AND THERAPEUTIC AGENTS

(75) Inventors: Douglas Kim GRAHAM, Aurora, CO (US); Susan Louise SATHER, Denver, CO (US)

(73) Assignee: THE REGENTS OF THE UNIVERSITY OF COLORADO, a body corporate, Denver, CO (US)

(21) Appl. No.: 13/230,005

(22) Filed: Sep. 12, 2011

Related U.S. Application Data
(62) Division of application No. 11/720,185, filed on Apr. 7, 2008, now abandoned.
(60) Provisional application No. 60/630,192, filed on Nov. 24, 2004.

(51) Int. Cl. G01N 33/574; C12Q 1/48

(52) U.S. Cl. 435/7.23; 435/15

(57) ABSTRACT

Mer diagnostic and therapeutic agents are disclosed. The agents are useful in the diagnosis and treatment of a variety of diseases including leukemia, lymphoma, and clotting disorders.

### CROSS-REFERENCE TO RELATED APPLICATIONS

This application is a divisional of U.S. patent application Ser. No. 11/720,185, filed Apr. 7, 2008, which is a national stage application under 35 U.S.C. §371 of PCT Application Serial No. PCT/US2005/042724, filed Nov. 23, 2005, which published in English, and which claims the benefit of priority under 35 U.S.C. §119(e) from U.S. Provisional Application Ser. No. 60/630,192, filed Nov. 24, 2004. Each of these applications is incorporated herein by reference in their entirety.

### FIELD OF THE INVENTION

The present invention relates to compositions useful for the diagnosis and treatment of disorders associated with the Mer transmembrane receptor tyrosine kinase.

### BACKGROUND OF THE INVENTION

Tyrosine kinases play an important role in normal cellular growth and differentiation. Deregulation of tyrosine kinase activity can result in cellular transformation leading to the development of human cancer. Mer is a transmembrane receptor tyrosine kinase that is likely the human homologue of the chicken retroviral gene, v-eyk, which causes many types of cancer in chicken. The human Mer gene and the mouse Mer gene and cDNA have been sequenced and characterized, and the expression of Mer has been profiled in cell lines and tissues (Graham et al., Cell Growth and Differentiation, 1994, 5:647-657; Graham et al., Oncogene, 1995, 10:2349-2359; and U.S. Pat. No. 5,585,269). The Mer receptor tyrosine kinase, initially cloned from a human B lymphoblastoid cell line, is expressed in a spectrum of hematopoietic, epithelial, and mesenchymal cell lines. Interestingly, while the RNA transcript of Mer is detected in numerous T and B lymphoblastic cell lines, Mer RNA is not found in normal human thymocytes, lymphocytes or in PMA/PHA stimulated lymphocytes. Mer is composed of two immunoglobulin domains and two fibronectin III domains in the extracellular portion, and a tyrosine kinase domain in the intracellular portion (Graham et al., (1994), supra and Graham et al., (1995), supra). Human Mer is known to be transforming and anti-apoptotic, and Mer overexpression has been linked to a number of different human cancers including subsets of B and T cell leukemia, lymphoma, pituitary adenoma, gastric cancer, and rhabdomyosarcoma.

Mer is related to two other receptor tyrosine kinases, Axl and Tyro-3. Mer, Axl, and Tyro-3 are all expressed in a spectrum of hematopoeitic, epithelial, and mesenchymal cell lines. Each protein has been shown to have the capability to transform cells in vitro. The overexpression of Mer leads to the transformation of NIH 3T3 cells and Ba/F3 pro lymphocytes (Ling et al., Mol. Cell Bio. 15:6582-6592 (1995) and Georgescu et al., Mol. Cell Bio. 19:1171-1181 (1999)). Axl, originally identified as a protein encoded by a transforming gene from primary human myeloid leukemia cells, is overexpressed in a number of different tumor cell types and transforms NIH3T3 fibroblasts (O'Bryan et al., Mol. Cell Bio. 11:5016-5031 (1991)). Tyro-3 is expressed at elevated levels in mammary tumors (Taylor et al., J. Biol. Chem., 270:6872-6880 (1995)) and its overexpression causes transformed growth of fibroblasts (Lai et al., Oncogene 9:2567-2578 (1995)).

Within hematopoietic cell lines, the Mer receptor tyrosine kinase is normally expressed in monocytes/macrophages, dendritic cells, megakaryocytes, and platelets. Mer RNA transcript or protein is not detected in lymphocytes or thymocytes. However, in acute lymphoblastic leukemia cell lines and patient samples, Mer RNA transcript and protein is present (Graham et al., (1994), supra; Graham et al., (1995), supra; and U.S. Pat. No. 5,585,269).

Mer, Axl, and Tyro-3 are all activated by the ligand Gas6. Gas6 is structurally similar to Protein S, a cofactor for anticoagulant Protein C, and shares 48% protein identity with Protein S. Gas6 plays a role in coagulation (Angelillo-Scherrer et al., Nature Medicine 7:215-21 (2002)). Gas6 antibodies may be used to protect wild type mice against fatal thromboembolism (Angelillo-Scherrer et al., (2002)). Mice with an inactivated Gas6 and/or Mer gene have platelet dysfunction that prevents venous and arterial thrombosis. These knockout mice are protected against fatal collagen/epinephrine induced thromboembolism and inhibited ferric chloride-induced thrombosis in vivo. Gas6 amplifies platelet aggregation and secretion response of platelets to known agonists (Chen et al., Aterioscler. Thromb. Vasc. Biol. 24:1118-1123 (2004)). The platelet dysfunction caused by Gas6 is thought to be mediated through the Mer, Axl, or Tyro-3. Thus, the Mer receptor tyrosine kinase may play a significant role in the development of hemostasis and human cancer.

Various types of thrombosis and the complications associated with thrombosis represent a major cause of morbidity and death in the world. Malignant cellular growth or tumors (cancer) are also a leading cause of death worldwide. The development of effective therapy for cardiovascular and neoplastic disease is the subject of a large body of research. Although a variety of innovative approaches to treat and prevent such diseases have been proposed, these diseases continue to have a high rate of mortality and may be difficult to treat or relatively unresponsive to conventional therapies. Therefore, there is a continued need in the art for new therapies that can effectively target and prevent or treat these diseases.

### SUMMARY OF THE INVENTION

One embodiment of the present invention relates to a method of diagnosing leukemia or lymphoma. The method includes detecting aberrant glycoforms of Mer transmembrane receptor tyrosine kinase (Mer) in lymphocytes from an individual, wherein the expression of aberrant glycoforms of the Mer transmembrane receptor tyrosine kinase by lymphocytes in the individual is indicative of leukemia or lymphoma. The individual is preferably a mammal, and more preferably, a human.

In one aspect, the method comprises detecting an aberrant glycoform of Mer selected from: a Mer glycoform having a molecular weight between about 170 kD and about 190 kD, a Mer glycoform having a molecular weight between about 135 kD and about 140 kD, and a Mer glycoform having a molecular weight between about 190 kD and about 195 kD. In one aspect, the leukemia is a lymphoblastic leukemia, and wherein the method includes the detection of at least one Mer glycoform having a molecular weight of between about 170 kD and about 190 kD, or having a molecular weight between about 135 kD and about 140 kD. In another aspect, a Mer glycoform having a molecular weight of between about 170 kD and about 190 kD is detected, and the method further comprises detecting the amount of the Mer glycoform expressed by the cells, wherein expression of Mer and lack of CD3 expression by the cells, indicates a poor prognosis for the individual. In one aspect, the leukemia is a myelogenous leukemia or lymphoma, and the method includes the detection of a Mer glycoform having a molecular weight of between about 190 kD and about 195 kD.

In this method of the invention, the step of detection can include contacting the sample with an antibody or antigen binding fragment thereof that selectively binds to said Mer transmembrane receptor tyrosine kinase glycoform.

Another embodiment of the present invention relates to a method for the production of a monoclonal antibody that selectively binds to a specific glycoform of the Mer transmembrane receptor tyrosine kinase comprising: (a) immunizing a non-human mammal with a glycosylated Mer transmembrane receptor tyrosine kinase such that antibody-producing cells are produced, wherein the glycosylated Mer transmembrane receptor tyrosine kinase is at least an extracellular portion of a full-length Mer transmembrane receptor tyrosine kinase, wherein the full-length Mer transmembrane receptor tyrosine kinase has a molecular weight of less than about 195 kD; (b) removing and immortalizing said antibody producing cells; (c) selecting and cloning the immortalized antibody producing cells producing the desired antibody; and (d) isolating the antibodies produced by the selected, cloned immortalized antibody producing cells. In one aspect, the Mer transmembrane receptor tyrosine kinase is selected from the group consisting of: a Mer glycoform having a molecular weight between about 165 kD and about 170 kD, a Mer glycoform having a molecular weight between about 170 kD and about 190 kD, a Mer glycoform having a molecular weight between about 135 kD and about 140 kD, and a Mer glycoform having a molecular weight between about 190 kD and about 195 kD. In another aspect, the Mer transmembrane receptor tyrosine kinase is expressed by a cell type selected from the group consisting of: platelets, lymphoblastic leukemia cells, and myelogenous leukemia cells. Included in this embodiment is any antibody produced by the method, including a monoclonal antibody.

Yet another embodiment of the present invention relates to an isolated antibody that selectively binds to a glycosylated Mer transmembrane receptor tyrosine kinase protein, wherein the antibody detects any glycosylation form of the Mer protein.

Another embodiment of the invention relates to an isolated antibody that selectively binds to a Mer transmembrane receptor tyrosine kinase glycoform (Mer glycoform) selected from: a Mer glycoform having a molecular weight between about 195 kD and about 210 kD, a Mer glycoform having a molecular weight between about 165 kD and about 170 kD, a Mer glycoform having a molecular weight between about 170 kD and about 190 kD, a Mer glycoform having a molecular weight between about 135 kD and about 140 kD, and a Mer glycoform having a molecular weight between about 190 kD and about 195 kD.

In one aspect, any of the above-identified antibodies selectively bind to all of the Mer glycoforms selected from the group consisting of: a Mer glycoform having a molecular weight between about 195 kD and about 210 kD, a Mer glycoform having a molecular weight between about 165 kD and about 170 kD, a Mer glycoform having a molecular weight between about 170 kD and about 190 kD, a Mer glycoform having a molecular weight between about 135 kD and about 140 kD, and a Mer glycoform having a molecular weight between about 190 kD and about 195 kD. In another aspect, the antibody selectively binds to one of said Mer glycoforms and not to the other Mer glycoforms.

Another embodiment of the present invention relates to an isolated antibody that selectively binds to a Mer transmembrane receptor tyrosine kinase glycoform (Mer glycoform), wherein the Mer glycoform has a molecular weight of less than about 195 kD. In one aspect, the Mer glycoform has a molecular weight of between about 170 kD and about 190 kD or less than about 160 kD. In another aspect, the Mer glycoform has a molecular weight of between about 170 kD and about 190 kD or between about 135 kD and about 140 kD. In yet another aspect, the Mer glycoform has a molecular weight of between about 190 kD and about 195 kD. In yet another aspect, the antibody selectively binds to a Mer glycoform expressed by leukemia or lymphoma cells. In yet another aspect, the antibody selectively binds to a Mer glycoform expressed by lymphoblastic leukemia cells. In yet another aspect, the antibody selectively binds to a Mer glycoform expressed by myelogenous leukemia cells.

Another embodiment of the invention includes a composition comprising any of the above-identified antibodies. In one aspect, the composition further comprises a pharmaceutically acceptable carrier.

Another embodiment of the invention includes the use of any of the above-identified antibodies in a composition for diagnosing leukemia or lymphoma.

Yet another embodiment of the invention includes the use of any of the above-identified antibodies in a pharmaceutical formulation for treating a cancer in an individual.

Another embodiment of the present invention relates to a method of treating cancer in an individual positive for surface expression of a Mer glycoform, comprising administering to the individual an antibody that selectively binds to the Mer glycoform and inhibits the activity of the Mer glycoform. In one aspect, the cancer is a leukemia or lymphoma. In another aspect, the Mer glycoform is selected from the group consisting of: a Mer glycoform having a molecular weight between about 170 kD and about 190 kD, a Mer glycoform having a molecular weight between about 135 kD and about 140 kD, and a Mer glycoform having a molecular weight between about 190 kD and about 195 kD. In one aspect, the antibody is any of the above-described antibodies.

Yet another embodiment of the present invention relates to a method of treating or preventing a clotting disorder in an individual, comprising administrating to the individual a therapeutically effective amount of an antibody that selectively binds to and inhibits the activity of a Mer glycoform having a molecular weight of between about 165 kD and about 170 kD.

Another embodiment of the present invention relates to a method of treating or preventing a clotting disorder in an individual, comprising administrating to the individual an effective amount of a soluble glycoform of a Mer transmembrane receptor tyrosine kinase, wherein the soluble Mer glycoform is glyosylated in a manner similar to the glycoform of Mer present on platelets. In one aspect, the soluble Mer glycoform has a molecular weight of between about 165 kD and about 170 kD.

In either of the above-identified embodiments, the clotting disorder can include, but is not limited to, thrombophilia.

Another embodiment of the present invention relates to an isolated soluble Mer transmembrane receptor tyrosine kinase (Mer) protein, wherein the soluble Mer protein is glycosylated, wherein the soluble Mer protein binds to a Mer ligand, and wherein the soluble Mer protein is a soluble portion of a full-length Mer glycoform or a homologue thereof having a molecular weight of less than about 195 kD. In one aspect, the full-length Mer glycoform is a Mer glycoform expressed by platelets. In another aspect, the full-length Mer glycoform is a Mer glycoform having a molecular weight of from about 165 kD to about 170 kD. In another aspect, the full-length Mer glycoform has a molecular weight of between about 170 kD and about 190 kD or less than about 160 kD. In yet another aspect, the full-length Mer glycoform is a Mer glycoform expressed by a leukemia or lymphoma cell. In yet another aspect, the full-length Mer glycoform is a Mer glycoform expressed by a lymphoblastic leukemia cell. In this aspect, the full-length Mer glycoform can have a molecular weight of between about 170 kD and 190 kD or between about 135 kD and about 140 kD. In another aspect, the full-length Mer glycoform is a Mer glycoform expressed by a myelogenous leukemia cell. In this aspect, the full-length Mer glycoform can have a molecular weight of between about 190 kD and about 195 kD. In yet another aspect, the full-length Mer glycoform comprises an amino acid sequence that is at least about 90% identical to SEQ ID NO:2. In another aspect, the soluble portion is an extracellular portion of the full-length Mer glycoform that binds to the Mer ligand. The Mer ligands include, but are not limited to, Gas6 and Protein S. In yet another aspect, the soluble Mer transmembrane receptor tyrosine kinase binds to Protein S and not to Gas6. In another aspect, the soluble Mer transmembrane receptor tyrosine kinase binds to Protein S with a higher affinity than to Gas 6.

Yet another embodiment of the present invention relates to a fusion protein, comprising any of the above-described isolated soluble Mer transmembrane receptor tyrosine kinases, linked to a heterologous protein. In one aspect, the heterologous protein is an Fc fragment of an immunoglobulin protein.

Another embodiment of the invention relates to the use of any of the above-identified isolated soluble Mer transmembrane receptor tyrosine kinases or fusion proteins in a pharmaceutical formulation.

Another embodiment of the invention relates to the use of certain of the above-identified isolated soluble Mer transmembrane receptor tyrosine kinases or fusion proteins in a pharmaceutical formulation for treating a cancer.

Yet another embodiment of the invention relates to the use of certain of the above-identified isolated soluble Mer transmembrane receptor tyrosine kinases or fusion proteins in a pharmaceutical formulation for treating a clotting disorder.

Another embodiment of the present invention relates to a method of treating cancer in an individual positive for surface expression of a Mer glycoform in cancer cells, comprising administrating to the individual a soluble form of the Mer glycoform or a fusion protein comprising the soluble form of the Mer glycoform, including the above identified soluble Mer transmembrane receptor tyrosine kinases.

Yet another embodiment of the present invention relates to a method of treating or preventing a clotting disorder in an individual comprising administering to the individual a soluble form of a Mer glycoform having a molecular weight of between about 165 kD and about 170 kD, or a fusion protein comprising the soluble form of the Mer glycoform.

Another embodiment of the present invention relates to a method of treating or preventing a clotting disorder in an individual comprising administering to the individual an agent which modulates the cleavage of the extracellular domain of the Mer transmembrane receptor tyrosine kinase. In one aspect, the agent inhibits cleavage of the extracellular domain of the Mer transmembrane receptor tyrosine kinase, and can include, but is not limited to, a TACE inhibitor. In another aspect, the agent cleaves the extracellular domain of the Mer transmembrane receptor tyrosine kinase, and can include, but is not limited to, a TACE-like metalloprotease.

In one aspect of the above-described methods related to clotting disorders, the disorder is thrombophilia.

Yet another embodiment of the present invention relates to a method of screening for compounds that regulate blood clotting comprising: (a) contacting a putative regulatory compound with cells expressing a Mer transmembrane receptor tyrosine kinase; and (b) detecting compounds that cleave an extracellular fragment of the Mer transmembrane receptor kinase into the medium as compared to cells not in contact with said compound. In one aspect, the cells are platelets. In another aspect, the compound is a cleavage agent.

Another embodiment of the invention relates to a method for screening for compounds that modulate the activity of a specific glycoform of Mer transmembrane receptor tyrosine kinase comprising: (a) contacting a putative regulatory compound with a Mer transmembrane receptor tyrosine kinase, wherein the Mer transmembrane receptor tyrosine kinase is a glycoform of Mer selected from the group consisting of: a Mer glycoform having a molecular weight between about 195 kD and about 210 kD and a Mer glycoform having a molecular weight of less than about 195 kD; and (b) detecting compounds that selectively bind to the Mer glycoform. In one aspect, the Mer glycoform is selected from: a Mer glycoform having a molecular weight between about 195 kD and about 210 kD, a Mer glycoform having a molecular weight between about 165 kD and about 170 kD, a Mer glycoform having a molecular weight between about 170 kD and about 190 kD, a Mer glycoform having a molecular weight between about 135 kD and about 140 kD, and a Mer glycoform having a molecular weight between about 190 kD and about 195 kD. In one aspect, the Mer glycoform has a molecular weight of between about 170 kD and about 195 kD or less than about 160 kD. In another aspect, the Mer glycoform is a Mer glycoform expressed by lymphoblastic leukemia cells. In this aspect, the Mer glycoform can have a molecular weight of between about 170 kD and about 190 kD, or a molecular weight of between about 135 kD and about 140 kD. In another aspect, the Mer glycoform is a Mer glycoform expressed by myelogenous leukemia cells. In this aspect, the Mer glycoform can have a molecular weight of between about 190 kD and about 195 kD.

In any of the above screening methods, in one aspect, the step of detecting comprises a step of detecting compounds that bind to one Mer glycoform and not to another Mer glycoform. In another aspect, the Mer glycoform is expressed by a cell. In yet another aspect, the Mer glycoform is a soluble Mer glycoform. In yet another aspect, the method further comprises detecting whether the compound inhibits the binding of a Mer ligand to Mer expressed by a cell. For example, a Mer ligand can include, but is not limited to, Gas6 and Protein S. In another aspect, the method further includes detecting compounds that inhibit the binding of Protein S to Mer expressed by a cell and do not inhibit the binding of Gas6 to Mer expressed by a cell. In one aspect, the method further comprises detecting whether the compound inhibits the activity of Mer expressed by a cell. In another aspect, the compound is selected from the group consisting of: a small molecule, a nucleic acid, a protein, a peptide and an antibody. In another aspect, the compound is a soluble Mer protein. In yet another aspect, such a soluble Mer protein is a different glycoform than the Mer transmembrane receptor tyrosine kinase of step (a).

### BRIEF DESCRIPTION OF THE DRAWINGS

FIG. 1 is a Western Blot illustrating that the monoclonal antibody 311 directed against human Mer recognizes a 205 kD protein in the monocytic cell line U937 and a 185 kD protein in the Jurkat leukemia cell line.

FIG. 2 is a Western blot showing that the human Mer 185 kD protein in leukemia cell lines is distinct from Mer in other hematopoietic lineages.

FIG. 3 is a Western blot confirming through deglycosylation that the Jurkat leukemia cell line expresses unique Mer proteins.

FIG. 4 is a Western blot illustrating detection of two different glycosylation forms of Mer, 185 kD and 140 kD, in leukemia cell lines and T cell acute lymphoblastic leukemia (ALL) patient samples.

FIGS. 5A-5C are Western blots showing that mutations/truncations of Mer protein exist in some leukemia patient samples.

FIG. 6 is a Western blot illustrating that both Gas6 and Protein S are ligands for Mer.

FIGS. 7A-7D are a Western blots showing that soluble Mer is shed into the medium of cultured cells.

FIGS. 8A-8D are flow cytometry (FIGS. 8A-8B) and Western blots (FIGS. 8C-8D) demonstrating that LPS and PMA induce cleavage (shedding) of the Mer extracellular domain.

FIG. 9 is a Western blot showing that soluble Mer is shed into the medium by cultured mouse macrophage and spleen cells and is present in mouse blood.

FIG. 10 is a Western blot illustrating that soluble Mer is present in human blood.

FIG. 11 is a Western blot showing that a specific metalloprotease inhibitor (TAPI) blocks production of soluble Mer.

FIGS. 12A and 12B are a schematic drawing (FIG. 12A) and a Western blot (FIG. 12B) indicating that soluble Mer (Mer/Fc) binds to Gas6.

FIGS. 13A and 13B are Western blots illustrating that soluble Mer (Mer/Fc) inhibits Gas6 signaling.

FIG. 14 is a Western blot demonstrating that soluble Mer (Mer/Fc) is glycosylated differently in mammalian and insect cells.

FIG. 15 is a graph illustrating that Mer expression is associated with the lack of surface CD3 on lymphoblasts.

FIGS. 16A-16D are platelet aggregometer traces showing that sMer (Mer/Fc) inhibits platelet aggregation.

FIG. 17 is a graph illustrating that sMer (Mer/Fc) protects against fatal thromboembolism.

FIG. 18 is a Western blot demonstrating that Mer activation leads to the activation of downstream pro-survival pathways AKT and ERK ½.

FIGS. 19A-19F are digital images of Mer transgenic mice and histological tissues demonstrating the presence of lymphoblastic leukemia/lymphoma and flow cytometry confirming that the Mer positive tumors are T cell in origin.

### DETAILED DESCRIPTION OF THE INVENTION

The present invention generally relates to the present inventors' discovery that unique glycoforms of Mer, also known as c-Mer, Mertk, or Mer receptor tyrosine kinase, are expressed by different cell types. In particular, the present inventors have discovered that Mer expressed by monocytes and macrophages can be distinguished from Mer expressed by platelets on the basis of the glycosylation of the extracellular domain of the receptor, and furthermore, that additional unique Mer glycoforms are expressed by different types of tumor cells. For example, in addition to the ectopic expression of Mer in certain cancers, including leukemia and lymphoma, the Mer extracellular domain in cancer cells (e.g., leukemia and lymphoma cells), is glycosylated in a manner that is different from the glycosylation present on Mer found in normal platelets and monocytes/macrophages. Moreover, different types of leukemia cells express different Mer glycoforms. The discovery of the presence of multiple unique Mer glycoforms that are differentially associated with cell type has significant diagnostic and therapeutic potential. For example, a specific glycoform of Mer can be used as a marker to identify a high risk leukemia or lymphoma patient that should be classified differently from other leukemia or lymphoma patients. In addition, therapeutic strategies can be designed to target a particular Mer glycoform and thus, a particular cell type or tumor type.

In addition to the unique Mer glycoforms present in various cell types as described herein, the present inventors have also detected variants (mutations or Mer protein alterations) in leukemia patient samples (e.g., resulting from mutations, deletions, or alternative splicing of the Mer gene). Such Mer mutants or splice variants (collectively referred to as Mer variants) can also be used to design additional diagnostic and therapeutic agents for use in the various methods described herein.

Therefore, in addition to the use of Mer as a diagnostic or prognostic marker, the unique Mer glycoforms and Mer variants discovered by the present inventors are valuable therapeutic targets for drug discovery and/or for in vivo treatments related to cancer or thrombosis. For example, small molecule inhibitors or drugs targeting glycoform-specific Mer proteins, or proteins directly downstream of Mer expressed by specific cell types, can be engineered and used in treatment regimens for such diseases. The Mer glycoforms can further be used to develop novel agents, such as specifically glycosylated soluble Mer (sMer) proteins, fusion proteins and chimeric proteins, that can be used in diagnostic or therapeutic methods. For example, the present invention encompasses the production and use of Mer glycoforms that are competitive inhibitors of Mer proteins that are endogenously expressed by particular cell types.

The present invention also relates to novel antibodies or antigen-binding fragments thereof that are capable of recognizing different glycoforms and protein variants of Mer protein, such as the aberrant glycoforms expressed by leukemia cells and protein variants within this glycoform. These antibodies are also useful as diagnostic and/or therapeutic reagents. Other embodiments of the present invention will be apparent to those of skill in the art from the description provided herein.

### Mer Glycoforms

Accordingly, one embodiment of the present invention relates to the recognition of unique, cell type-specific glycoforms of Mer, and the use of these glycoforms to design novel diagnostic and therapeutic agents (e.g., antibodies, soluble Mer proteins, etc.) that relate to these glycoforms, as well as methods for using these agents and methods that take advantage of the discovery of the various Mer glycoforms. Specifically, the present inventors have discovered that several different glycosylation forms of the Mer protein are expressed by cells in a cell-type-dependent manner. Such glycosylation forms (also referred to as “Mer glycoforms”) include, but are not limited to: a fully glycosylated 195-210 kD Mer protein that is expressed by monocytic cells (e.g., monocytes and macrophages), which can be detected as any value between about 195 kD and about 210 kD, inclusive, such as between about 205 kD and about 210 kD, or about 205 kD, and several less-glycosylated Mer proteins (Mer proteins having less glycosylation than the monocytic cell types) that are expressed by other cell types. Glycoforms of Mer that are less than the fully glycosylated form expressed by monocytic cell types include, but are not limited to: (1) a glycoform expressed by platelets, which is from about 165 kD to about 170 kD; (2) two distinct glycoforms expressed by Mer lymphoblastic leukemias, including a glycoform found on acute lymphoblastic leukemia (ALL) cell lines that is between approximately 170 kD and approximately 190 kD (including from about 170 kD to about 180 kD or about 185 kD) and/or from about 135 kD to about 140 kD; and (3) a novel Mer glycoform specific to chronic myelogenous leukemia detected at from about 190 kD to about 195 kD. Therefore, the present inventors have found that Mer glycoforms present in lymphoblastic leukemia or myelogenous leukemia, for example, differ from each other and from the forms of Mer found in monocytic cells and platelets.

The present invention makes use of Mer glycoforms that are normally (naturally, endogenously, constitutively) expressed by certain hematopoietic cells (e.g., monocytic cells or platelets) or by other cell types that are not transformed (not neoplastically transformed, cancerous, or displaying aberrant or abnormal growth), as well as “aberrantly glycosylated” Mer glycoforms that are expressed by cells that are displaying aberrant or abnormal growth (cancerous, neoplastically transformed) or that are becoming or are predisposed to becoming cancerous. According to the present invention, a “Mer glycoform” is a Mer receptor tyrosine kinase that is described or characterized by the level of glycosylation of the extracellular domain of the receptor, which reflects the molecular weight of the Mer protein, as determined by any suitable method known in the art (e.g., Western blot, other electrophoresis methods, chromatography, analytical ultracentrifugation, etc.). The glycosylation status of a Mer glycoform can be represented herein as a range of molecular weights (described above), which is reflective of potential differences in the determination of molecular weight, depending on the detection method used, standards used, and normal variation from assay to assay. A Mer glycoform can also be described more particularly by the available glycosylation sites on the protein and the number of such sites that are glycosylated, but will generally be referred to herein by the resulting molecular weight of the post-translationally modified protein. The terms “aberrantly glycosylated Mer”, “aberrantly glycosylated Mer glycoform” or “aberrant Mer glycoforms”, described in the present invention and defined herein refer to novel and unique Mer glycoforms that are expressed by cells that ectopically (abnormally) express Mer, such as cells displaying aberrant growth characteristics (e.g. cancer cells or neoplastically transformed cells, such as leukemia and lymphoma cells) or cells that are in the process of developing aberrant growth characteristics (e.g., precancerous cells). The term “aberrant Mer glycoform” is not used to describe Mer that is expressed by hematopoietic cell lineages that normally express Mer (e.g., monocytic cells and platelets). Aberrant Mer glycoforms include all glycoforms having molecular weights less than about 160 kD, and those forms between about 170 kD and about 190 kD. According to the present invention, the term “about” used in connection with the Mer glycoforms refers to a value that is plus or minus 2.5 kD.

### Mer Antibodies

One embodiment of the present invention relates to the development of novel anti-Mer antibodies, and particularly, novel anti-human Mer antibodies, and even more particularly, novel anti-human Mer monoclonal antibodies (mAb), any of which can be used in a variety of diagnostic and/or therapeutic methods as described below, as well as for the further study of the Mer expression, and particularly, ectopic Mer expression, in various cell types (e.g., leukemia cells, lymphoblasts). Also included in this embodiment are antigen-binding fragments of such antibodies. An exemplary monoclonal antibody of the present invention can detect, by any method (e.g., Western blot), the spectrum of Mer glycosylation states (Mer glycoforms) existing in normal human tissue and in human disease, or alternatively, selectively binds to a particular Mer glycoform and not to other Mer glycoforms. Antibodies of the present invention are useful for diagnostic applications in human cancer, thrombosis, and autoimmune disease. In the case of cancer, for example, since lymphoblastic leukemia and lymphoblastic lymphoma are considered to be different clinical manifestations of the same disease process, and are treated with similar chemotherapeutic regimes, this invention has a variety of diagnostic, prognostic, and therapeutic uses (e.g., in leukemias and non-Hodgkin's lymphomas). In addition, antibodies of the present invention are useful in therapeutic applications directed to similar conditions. Antibodies of the present invention are also useful as valuable research tools and for purification of Mer glycoforms of the invention.

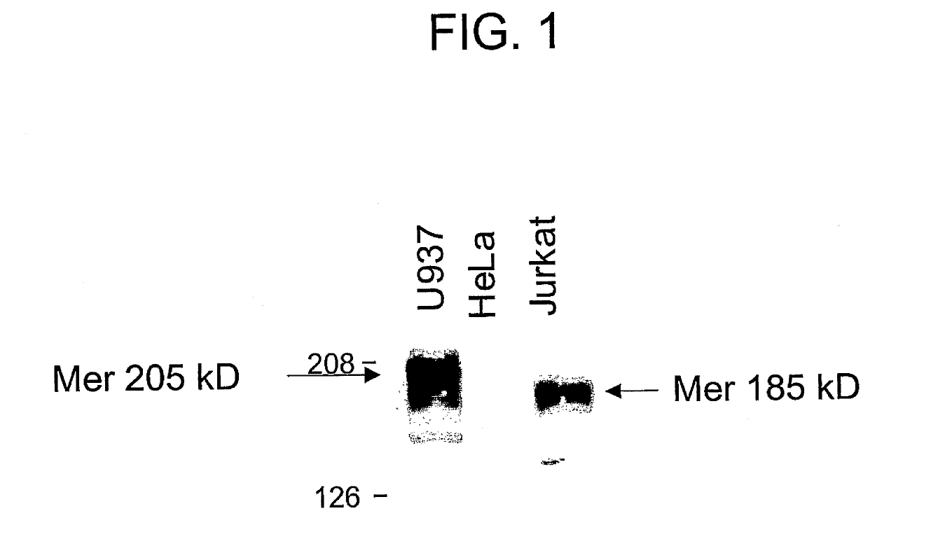

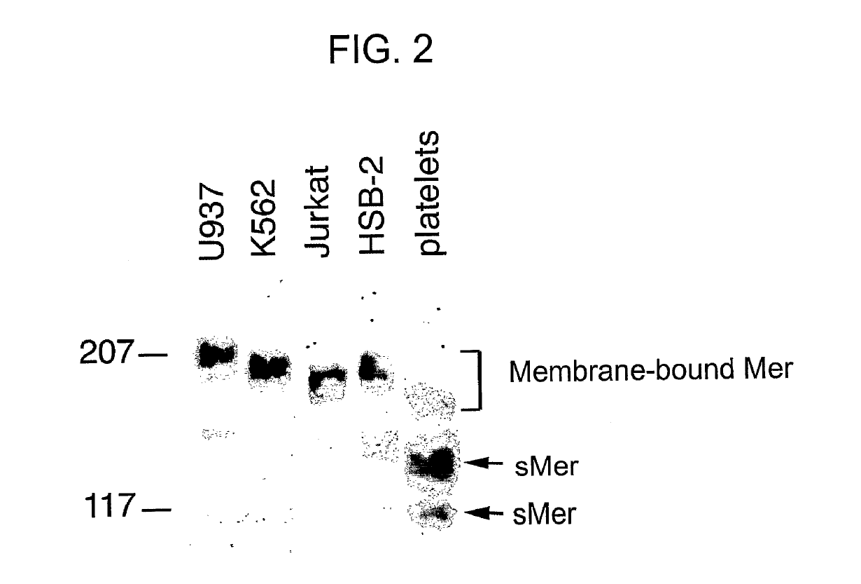

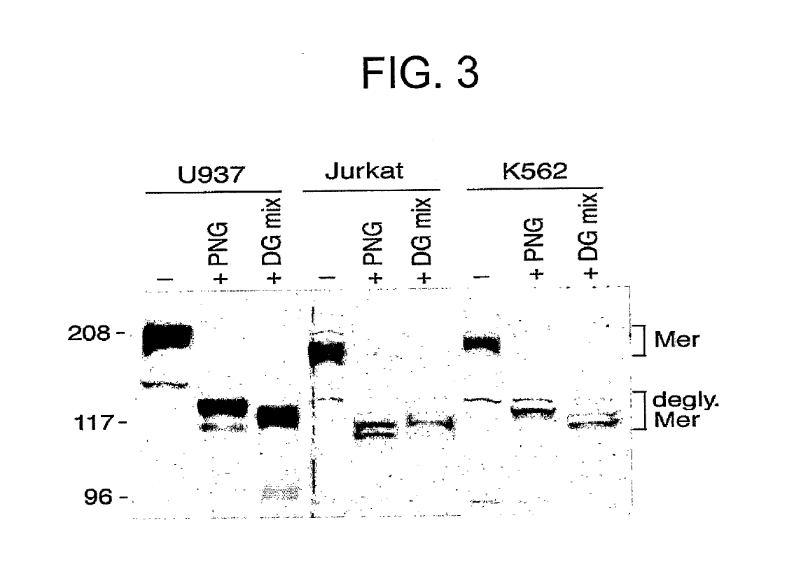

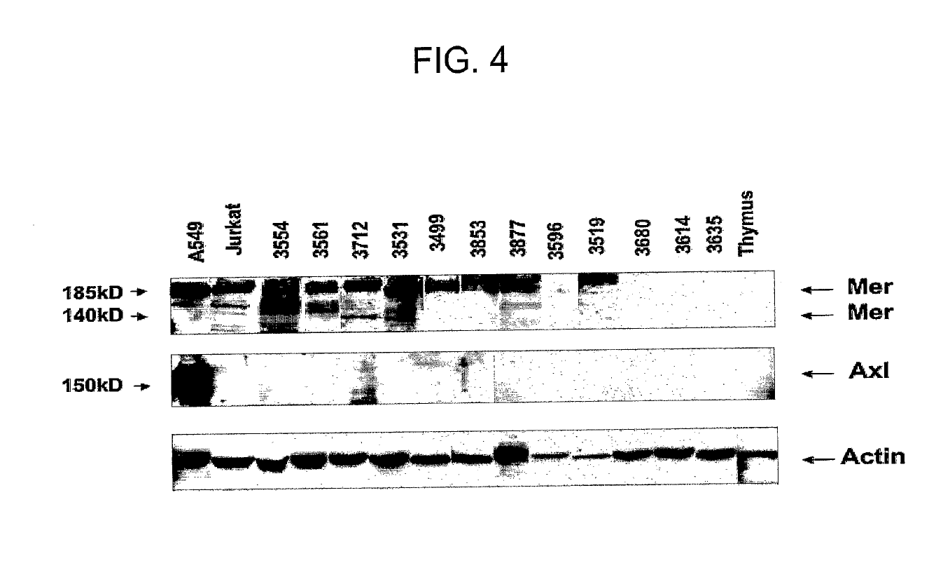

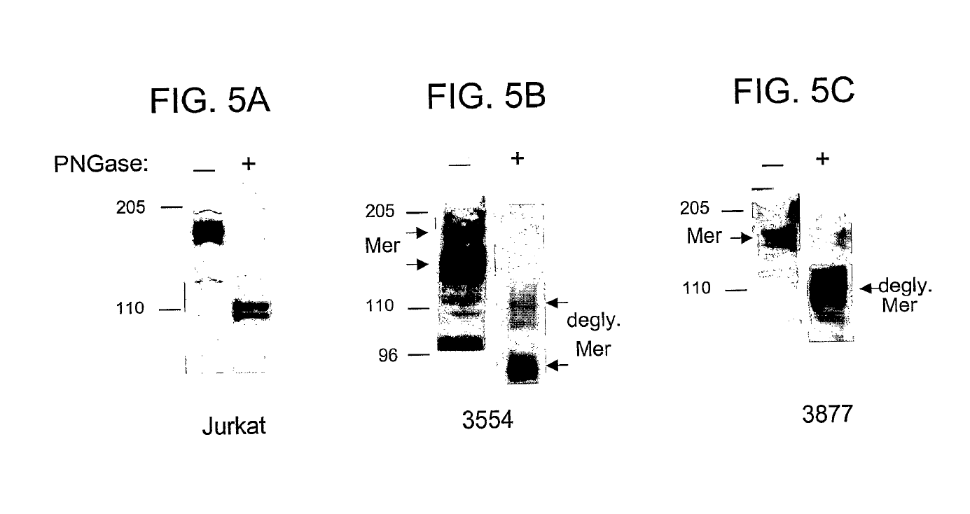

Accordingly, one embodiment of the present invention relates to anti-human Mer monoclonal antibodies that can detect the spectrum of Mer glycosylation states existing in human disease (i.e., both normal and aberrant Mer glycoforms) (FIG. 1), thereby allowing one to distinguish among the Mer glycoforms. To the best of the present inventors' knowledge, this is the first-described Mer antibody with such a specificity. For example, the anti-human Mer antibody referred to as the “311” antibody selectively recognizes multiple Mer glycoforms, including unique 170-190 kD forms of the protein in leukemia cell lines that are not present in other hematopoietic lineages (FIG. 2). The antibodies of the present invention are also capable of distinguishing among protein variants of Mer that do not differ in the glycosylation of the protein. For example, as described in the Examples, Western blots of samples treated with glycosidases to remove all N-linked and most O-linked carbohydrates from the glycoproteins revealed different Mer glycosylation patterns for leukemia cell lines when probed with Mer monoclonal antibody 311 (FIG. 3), further revealing the existence of distinct Mer protein variants within the unique Mer glycoform expressed by leukemia cells. Detection of the 170-190 kD glycoforms in T cell acute lymphoblastic leukemia (ALL) patient samples with the 311 antibody showed two different Mer glycoforms (170-190 kD and 135-145 kD) (FIG. 4). Furthermore, mutations or deletions resulting in potential differences at the glycosylation sites (in addition to glycosylation variation) can account for the differences in the size of the Mer protein as reflected in gel electrophoresis (FIG. 5). The discovery (through the utilization of the Mer 311 mAb as described above) that the Mer extracellular domain in leukemia cells that are resistant to common therapy is glycosylated in a manner different from the glycosylation present in platelets and monocytes/macrophages has diagnostic and therapeutic potential. The 311 monoclonal antibody is considered to be a prototypic antibody, and similar monoclonal antibodies to Mer (antibodies having the same or substantially similar specificity) are expected to show similar results. These similar monoclonal antibodies can be used to detect the presence of distinct Mer glycoforms in different cells types, such as in leukemia and lymphoma, by a variety of techniques, such as flow cytometry and Western blot, and can also be used to detect Mer-positive solid tumors, including lymphoma, by techniques such as immunohistochemistry (IHC) and Western blot.

Preferably, an antibody encompassed by the present invention includes any antibody that selectively binds to a conserved binding surface or epitope of a Mer protein, and preferably, to a conserved binding surface or epitope in the extracellular domain of the Mer protein (defined below). In one embodiment, an antibody of the present invention is capable of recognizing a spectrum of Mer glycoforms including a fully glycosylated Mer protein (an about 195 kD to about 210 kD Mer protein) as well as one or more other Mer glycoforms that are less than about 195 kD, and including aberrant Mer glycoforms as described above. Other Mer glycoforms can particularly include: a Mer glycoform that is from about 165 kD to about 170 kD; a Mer glycoform that is approximately 170 kD to about 190 kD (and more typically from about 170 kD to about 180 kD or more typically, about 185 kD), a Mer glycoform that is from about 135 kD to about 140 kD; and/or a Mer glycoform that is from about 190 kD to about 195 kD.

According to the present invention, an “epitope” of a given protein or peptide or other molecule is generally defined, with regard to antibodies, as a part of or a site on a larger molecule to which an antibody or antigen-binding fragment thereof will bind, and against which an antibody will be produced. The term epitope can be used interchangeably with the term “antigenic determinant”, “antibody binding site”, or “conserved binding surface” of a given protein or antigen. More specifically, an epitope can be defined by both the amino acid residues involved in antibody binding and also by their conformation in three dimensional space (e.g., a conformational epitope or the conserved binding surface). An epitope can be included in peptides as small as about 4-6 amino acid residues, or can be included in larger segments of a protein, and need not be comprised of contiguous amino acid residues when referring to a three dimensional structure of an epitope, particularly with regard to an antibody-binding epitope. Antibody-binding epitopes are frequently conformational epitopes rather than a sequential epitope (i.e., linear epitope), or in other words, an epitope defined by amino acid residues arrayed in three dimensions on the surface of a protein or polypeptide to which an antibody binds. As mentioned above, the conformational epitope is not comprised of a contiguous sequence of amino acid residues, but instead, the residues are perhaps widely separated in the primary protein sequence, and are brought together to form a binding surface by the way the protein folds in its native conformation in three dimensions.

As used herein, the term “selectively binds to” refers to the specific binding of one protein to another (e.g., an antibody, fragment thereof, or binding partner to an antigen), wherein the level of binding, as measured by any standard assay (e.g., an immunoassay), is statistically significantly higher than the background control for the assay. For example, when performing an immunoassay, controls typically include a reaction well/tube that contain antibody or antigen binding fragment alone (i.e., in the absence of antigen), wherein an amount of reactivity (e.g., non-specific binding to the well) by the antibody or antigen binding fragment thereof in the absence of the antigen is considered to be background. Binding can be measured using a variety of methods standard in the art, including, but not limited to: Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microarray, microscopy, fluorescence activated cell sorting (FACS), and flow cytometry.

One embodiment of the present invention includes an antibody or antigen binding fragment thereof that is a competitive inhibitor of the binding of a Mer ligand (e.g., Gas6 or Protein S) to a Mer glycoform that is expressed by a particular cell or cell type. In another embodiment, the present invention includes an antibody or antigen binding fragment thereof that is a competitive inhibitor of the binding of another anti-Mer antibody described herein (e.g., the 311 antibody). According to the present invention, a competitive inhibitor is an inhibitor (e.g., another antibody or antigen binding fragment or polypeptide) that binds to Mer that is expressed by a cell, and inhibits or blocks the binding of a natural Mer ligand (e.g., Gas6 or Protein S) to the Mer that is expressed by the cell. The antibody competitive inhibitor can also be defined by its ability to bind to Mer expressed by the cell at the same or similar epitope as another anti-Mer antibody described herein (e.g., mAb 311) such that binding of the anti-Mer antibody described herein is inhibited. A competitive inhibitor may bind to the target (e.g., Mer) with a greater affinity for the target than the Mer ligand or the other anti-Mer antibody. Other types of competitive inhibitors related to soluble Mer are described below. A competitive inhibitor can be used in a manner similar to that described herein for the anti-Mer antibody. Competition assays can be performed using standard techniques in the art (e.g., competitive ELISA or other binding assays). For example, competitive inhibitors can be detected and quantitated by their ability to inhibit the binding of Mer to another, labeled anti-Mer antibody.

Isolated antibodies of the present invention can include serum containing such antibodies, or antibodies that have been purified to varying degrees. Whole antibodies of the present invention can be polyclonal or monoclonal. Alternatively, functional equivalents of whole antibodies, such as antigen binding fragments in which one or more antibody domains are truncated or absent (e.g., Fv, Fab, Fab′, or F(ab)₂ fragments), as well as genetically-engineered antibodies or antigen binding fragments thereof, including single chain antibodies, humanized antibodies (discussed below), fully human antibodies, antibodies that can bind to more than one epitope (e.g., bi-specific antibodies), or antibodies that can bind to one or more different antigens (e.g., bi- or multi-specific antibodies), may also be employed in the invention.

Limited digestion of an immunoglobulin with a protease may produce two fragments. An antigen binding fragment is referred to as an Fab, an Fab′, or an F(ab′)₂ fragment. A fragment lacking the ability to bind to antigen is referred to as an Fc fragment. An Fab fragment comprises one arm of an immunoglobulin molecule containing a L chain (V_{L}+C_{L} domains) paired with the V_{H} region and a portion of the C_{H} region (CH1 domain). An Fab′ fragment corresponds to an Fab fragment with part of the hinge region attached to the CH1 domain. An F(ab′)₂ fragment corresponds to two Fab′ fragments that are normally covalently linked to each other through a di-sulfide bond, typically in the hinge regions.

The C_{H} domain defines the isotype of an immunoglobulin and confers different functional characteristics depending upon the isotype. For example, μ constant regions enable the formation of pentameric aggregates of IgM molecules and α constant regions enable the formation of dimers.

Other functional aspects of an immunoglobulin molecule include the valency of an immunoglobulin molecule, the affinity of an immunoglobulin molecule, and the avidity of an immunoglobulin molecule. As used herein, affinity refers to the strength with which an immunoglobulin molecule binds to an antigen at a single site on an immunoglobulin molecule (i.e., a monovalent Fab fragment binding to a monovalent antigen). Affinity differs from avidity which refers to the sum total of the strength with which an immunoglobulin binds to an antigen. Immunoglobulin binding affinity can be measured using techniques standard in the art, such as competitive binding techniques, equilibrium dialysis or BIAcore methods. As used herein, valency refers to the number of different antigen binding sites per immunoglobulin molecule (i.e., the number of antigen binding sites per antibody molecule of antigen binding fragment). For example, a monovalent immunoglobulin molecule can only bind to one antigen at one time, whereas a bivalent immunoglobulin molecule can bind to two or more antigens at one time, and so forth.

In one embodiment, the antibody is a bi- or multi-specific antibody. A bi-specific (or multi-specific) antibody is capable of binding two (or more) antigens, as with a divalent (or multivalent) antibody, but in this case, the antigens are different antigens (i.e., the antibody exhibits dual or greater specificity). For example, an antibody that selectively binds to Mer can be constructed as a bi-specific antibody, wherein the second antigen binding specificity is for a desired target, such as another cell surface marker on a target cell.

Antibodies of the present invention can include, but are not limited to, neutralizing antibodies, catalytic antibodies and blocking (binding) antibodies. According to the present invention, a neutralizing antibody is an antibody that reacts with an infectious agent (usually a virus) and destroys or inhibits its infectivity and virulence. A catalytic antibody is an antibody selected for its ability to catalyze a chemical reaction by binding to and stabilizing the transition-state intermediate. A blocking antibody is an antibody that binds to an antigen and blocks another antibody or agent from later binding to that antigen.

In one embodiment, antibodies of the present invention include humanized antibodies. Humanized antibodies are molecules having an antigen binding site derived from an immunoglobulin from a non-human species, the remaining immunoglobulin-derived parts of the molecule being derived from a human immunoglobulin. The antigen binding site may comprise either complete variable regions fused onto human constant domains or only the complementarity determining regions (CDRs) grafted onto appropriate human framework regions in the variable domains. Humanized antibodies can be produced, for example, by modeling the antibody variable domains, and producing the antibodies using genetic engineering techniques, such as CDR grafting (described below). A description various techniques for the production of humanized antibodies is found, for example, in Morrison et al. (1984) Proc. Natl. Acad. Sci. USA 81:6851-55; Whittle et al. (1987) Prot. Eng. 1:499-505; Co et al. (1990) J. Immunol. 148:1149-1154; Co et al. (1992) Proc. Natl. Acad. Sci. USA 88:2869-2873; Carter et al. (1992) Proc. Natl. Acad. Sci. 89:4285-4289; Routledge et al. (1991) Eur. J. Immunol. 21:2717-2725 and PCT Patent Publication Nos. WO 91/09967; WO 91/09968 and WO 92/113831.

In one embodiment, antibodies of the present invention include fully human antibodies. Fully human antibodies are fully human in nature. One method to produce such antibodies having a particular binding specificity includes obtaining human antibodies from immune donors (e.g., using EBV transformation of B-cells or by PCR cloning and phage display). In addition, and more typically, synthetic phage libraries have been created which use randomized combinations of synthetic human antibody V-regions. By selection on antigen, “fully human antibodies: can be made in which it is assumed the V-regions are very human like in nature. Phage display libraries are described in more detail below. Finally, fully human antibodies can be produced from transgenic mice. Specifically, transgenic mice have been created which have a repertoire of human immunoglobulin germline gene segments. Therefore, when immunized, these mice produce human like antibodies. All of these methods are known in the art.

Genetically engineered antibodies of the invention include those produced by standard recombinant DNA techniques involving the manipulation and re-expression of DNA encoding antibody variable and/or constant regions. Particular examples include, chimeric antibodies, where the V_{H} and/or V_{L} domains of the antibody come from a different source as compared to the remainder of the antibody, and CDR grafted antibodies (and antigen binding fragments thereof), in which at least one CDR sequence and optionally at least one variable region framework amino acid is (are) derived from one source and the remaining portions of the variable and the constant regions (as appropriate) are derived from a different source. Construction of chimeric and CDR-grafted antibodies are described, for example, in European Patent Applications: EP-A 0194276, EP-A 0239400, EP-A 0451216 and EP-A 0460617.

In one embodiment, chimeric antibodies are produced according to the present invention comprising antibody variable domains that bind to Mer and fused to these domains, a protein that serves as a second targeting moiety. For example, the targeting moiety can include a protein that is associated with the cell or tissue to be targeted or with a particular system in the animal.

In an additional embodiment, the present invention provides a method for the production of a monoclonal antibody that specifically binds to specific glycoforms of the Mer transmembrane receptor tyrosine kinase. Such an antibody can include any of the antibodies described herein, including, but not limited to, blocking or binding antibodies, neutralizing antibodies and catalytic antibodies. The method includes the steps of: (a) immunizing an animal with a specific Mer transmembrane receptor tyrosine kinase (i.e., a specific Mer glycoform) such that antibody producing cells are produced in the animal; (b) removing and immortalizing the antibody producing cells; (c) selecting and cloning the immortalized antibody producing cells producing the desired antibody; and (d) isolating the antibodies produced by the selected, cloned immortalized antibody producing cells. In one embodiment, the Mer transmembrane receptor tyrosine kinase used to produce the antibody is a soluble Mer (i.e., a fragment of Mer that comprises at least the portion of the extracellular domain of Mer that binds to its natural (cognate) ligands, such as Gas6 or Protein S.) Soluble Mer or extracellular domains of Mer are described in detail below. In a further embodiment, the invention provides for the monoclonal antibody produced by the above method.

Generally, in the production of an antibody, a suitable experimental animal, such as, for example, but not limited to, a rabbit, a sheep, a hamster, a guinea pig, a mouse, a rat, or a chicken, is exposed to an antigen against which an antibody is desired. Typically, an animal is immunized with an effective amount of antigen that is injected into the animal. An effective amount of antigen refers to an amount needed to induce antibody production by the animal. The animal's immune system is then allowed to respond over a pre-determined period of time. The immunization process can be repeated until the immune system is found to be producing antibodies to the antigen. In order to obtain polyclonal antibodies specific for the antigen, serum is collected from the animal that contains the desired antibodies (or in the case of a chicken, antibody can be collected from the eggs). Such serum is useful as a reagent. Polyclonal antibodies can be further purified from the serum (or eggs) by, for example, treating the serum with ammonium sulfate.

Monoclonal antibodies may be produced according to the methodology of Kohler and Milstein (Nature 256:495-497, 1975), or using the human B-cell hybridoma method, Kozbor, J., Immunol, 133:3001 (1984); Brodeur et al., Monoclonal Antibody Production Techniques and Applications, pp. 51-63 (Marcel Dekker, Inc., New York, 1987). For example, B lymphocytes are recovered from the spleen (or any suitable tissue) of an immunized animal and then fused with myeloma cells to obtain a population of hybridoma cells capable of continual growth in suitable culture medium. Hybridomas producing the desired antibody are selected by testing the ability of the antibody produced by the hybridoma to bind to the desired antigen. The hybridomas may be cloned and the antibodies may be produced by and then isolated from the hybridomas. A preferred method to produce antibodies of the present invention includes (a) administering to an animal an effective amount of a protein or peptide (e.g., a Mer protein or peptide including extracellular domains thereof) to produce the antibodies and (b) recovering the antibodies. As used herein, the term “monoclonal antibody” includes chimeric, humanized, and human forms of a monoclonal antibody. Monoclonal antibodies are often synthesized in the laboratory in pure form by a single clone (population) of cells. These antibodies can be made in large quantities and have a specific affinity for certain target antigens which can be found on the surface of cells. Monoclonal antibodies directed toward aberrant Mer glycoforms or a soluble form of the extracellular Mer receptor tyrosine kinase may be most easily produced using any of the well known methods that provides for the production of antibody molecules by continuous cell lines in culture.

In another method, antibodies of the present invention are produced recombinantly. For example, once a cell line, for example a hybridoma, expressing an antibody according to the invention has been obtained, it is possible to clone therefrom the cDNA and to identify the variable region genes encoding the desired antibody, including the sequences encoding the CDRs. From here, antibodies and antigen binding fragments according to the invention may be obtained by preparing one or more replicable expression vectors containing at least the DNA sequence encoding the variable domain of the antibody heavy or light chain and optionally other DNA sequences encoding remaining portions of the heavy and/or light chains as desired, and transforming/transfecting an appropriate host cell, in which production of the antibody will occur. Suitable expression hosts include bacteria, (for example, an E. coli strain), fungi, (in particular yeasts, e.g. members of the genera Pichia, Saccharomyces, or Kluyveromyces,) and mammalian cell lines, e.g. a non-producing myeloma cell line, such as a mouse NSO line, or CHO cells. In order to obtain efficient transcription and translation, the DNA sequence in each vector should include appropriate regulatory sequences, particularly a promoter and leader sequence operably linked to the variable domain sequence. Particular methods for producing antibodies in this way are generally well known and routinely used. For example, basic molecular biology procedures are described by Maniatis et al. (Molecular Cloning, Cold Spring Harbor Laboratory, New York, 1989); DNA sequencing can be performed as described in Sanger et al. (PNAS 74, 5463, (1977)) and the Amersham International plc sequencing handbook; and site directed mutagenesis can be carried out according to the method of Kramer et al. (Nucl. Acids Res. 12, 9441, (1984)) and the Anglian Biotechnology Ltd. handbook. Additionally, there are numerous publications, including patent specifications, detailing techniques suitable for the preparation of antibodies by manipulation of DNA, creation of expression vectors and transformation of appropriate cells, for example as reviewed by Mountain A and Adair, J R in Biotechnology and Genetic Engineering Reviews (ed. Tombs, M P, 10, Chapter 1, 1992, Intercept, Andover, UK) and in the aforementioned European Patent Applications.

Alternative methods, employing, for example, phage display technology (see for example, U.S. Pat. No. 5,969,108, U.S. Pat. No. 5,565,332, U.S. Pat. No. 5,871,907, U.S. Pat. No. 5,858,657, U.S. Pat. No. 5,223,409; Fuchs et al. Bio/Technology, 9:1370-1372 (1991); or Griffiths et al. EMBO J., 12:725-734 (1993)) or the selected lymphocyte antibody method of U.S. Pat. No. 5,627,052 may also be used for the production of antibodies and/or antigen fragments of the invention, as will be readily apparent to the skilled individual. For example, a monoclonal antibody to an aberrantly glycosylated Mer polypeptide can be identified and isolated by screening a recombinant combinatorial immunoglobulin library (e.g., an antibody phage display library) with the polypeptide to thereby isolate immunoglobulin library members that bind the polypeptide. Kits for generating and screening phage display libraries are well known and commercially available.

The Mer 311 monoclonal antibody, or other monoclonal antibodies produced with the polypeptides described above, can be used to isolate a Mer polypeptide including any Mer glycoform that is specifically recognized by the antibody, by standard techniques (such as affinity chromatography or immunoprecipitation). An antibody specific to (that selectively binds to) a Mer polypeptide or another peptide of the present invention can be used to detect Mer (e.g., in a cellular lysate, cell supernatant, or tissue sample) to evaluate the abundance of, pattern of expression of, glycosylation of, and variants of Mer. Antibodies can be used diagnostically to monitor protein levels in tissue as part of a clinical testing procedure, e.g., to determine the efficacy of a given treatment regimen or to select appropriate patients for a Mer-specific therapy. Furthermore, the presently claimed glycoforms, isoforms, or mutated Mer proteins may have increased tyrosine kinase activity that plays a role in oncogenesis. Thus in one aspect, the monoclonal antibodies of the invention, which recognize and bind or inactivate native, aberrant glycoforms of, or altered Mer protein in leukemia and lymphoma cells, may be used to treat Mer positive cancer patients. These embodiments of the invention are described in detail below.

Coupling the antibody to a detectable substance can facilitate detection. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

### Mer Proteins, Soluble Mer Proteins and Mer Protein Variants

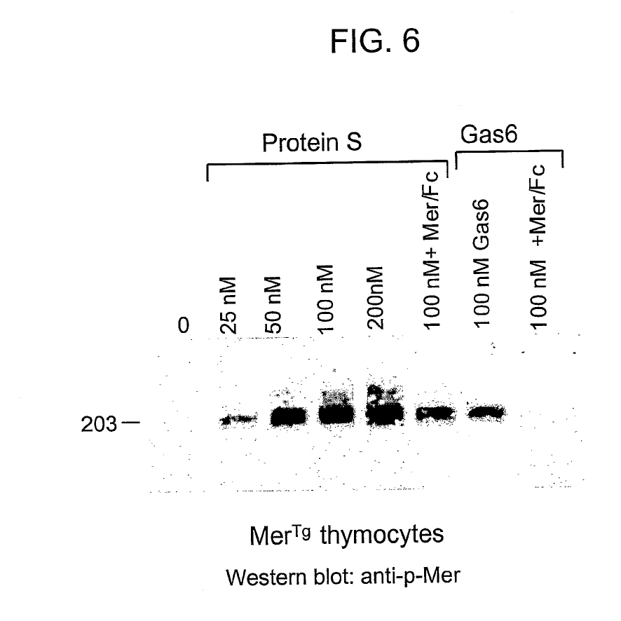

Another embodiment of the invention concerns the discovery that the Mer extracellular domain can be cleaved, and that this soluble portion of the Mer receptor tyrosine kinase is useful in inhibiting activation of the Mer receptor tyrosine kinase. The invention further contemplates novel, soluble forms of the Mer receptor tyrosine kinase present in human and mouse plasma that play a significant role in disease states or conditions where the Mer receptor tyrosine kinase is activated, such as cell survival signaling and cell proliferation in the case of Mer positive cancers, and in anticoagulation. While the inventors do not wish to be limited to their present theory as to how the invention operates, it is believed that the anticoagulation aspects of this invention function through the sMer interaction with its ligands including anticoagulant Protein S (FIG. 6) and Gas6. The novel, soluble Mer (sMer) proteins include specific glycoforms of sMer, particularly including the Mer glycoforms that have been specifically described herein. As used herein, the term “soluble form” of Mer, “sMer” or “soluble Mer” refers to a Mer receptor tyrosine kinase that is cleaved near the transmembrane receptor or that includes any portion of the extracellular domain of Mer (described below) that retains the ability to bind to a Mer ligand (e.g., Gas6 or Protein S). The exact cleavage site is not critical. Alternatively, the soluble form can be generated by differential splicing, by recombinant means, or by post-translational proteolytic cleavage, as described in additional detail below. A “soluble Mer glycoform” or “sMer glycoform” refers to a soluble form of Mer as described above that is additionally characterized by its particular glycosylation pattern or level of glycosylation.

As discussed above, the nucleic acid sequence (genomic or mRNA) and amino acid sequence for Mer from several different species are known in the art (e.g., see U.S. Pat. No. 5,585,269). The nucleic acid sequence for human Mer (mRNA) is represented herein by SEQ ID NO:1. SEQ ID NO:1 encodes human Mer protein, represented herein by SEQ ID NO:2. The nucleic acid sequence for murine Mer and the amino acid sequence of the protein encoded thereby are represented by SEQ ID NO:3 and SEQ ID NO:4, respectively. The nucleic acid sequence for rat Mer and the amino acid sequence of the protein encoded thereby are represented by SEQ ID NO:5 and SEQ ID NO:6, respectively. The nucleic acid sequence for chicken Mer and the amino acid sequence of the protein encoded thereby are represented by SEQ ID NO:7 and SEQ ID NO:8, respectively. The extracellular domain of human Mer (SEQ ID NO:2) spans amino acid positions from about 1 to about 473. The corresponding domain in other species can be readily determined by aligning the sequences. However, as discussed above, one may produce a soluble Mer that is cleaved at a site other than position 484 of SEQ ID NO:2 (or the corresponding position in Mer from other species). For example, soluble Mer proteins of the invention can include any smaller portions (fragments) of the extracellular domain of Mer that retain the ability to bind to a Mer ligand (e.g., Gas6 or Protein S).

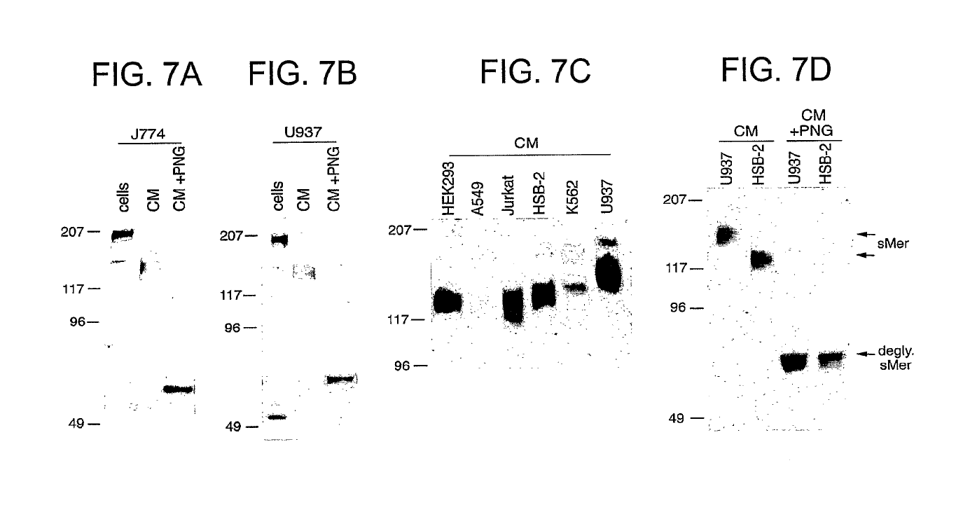

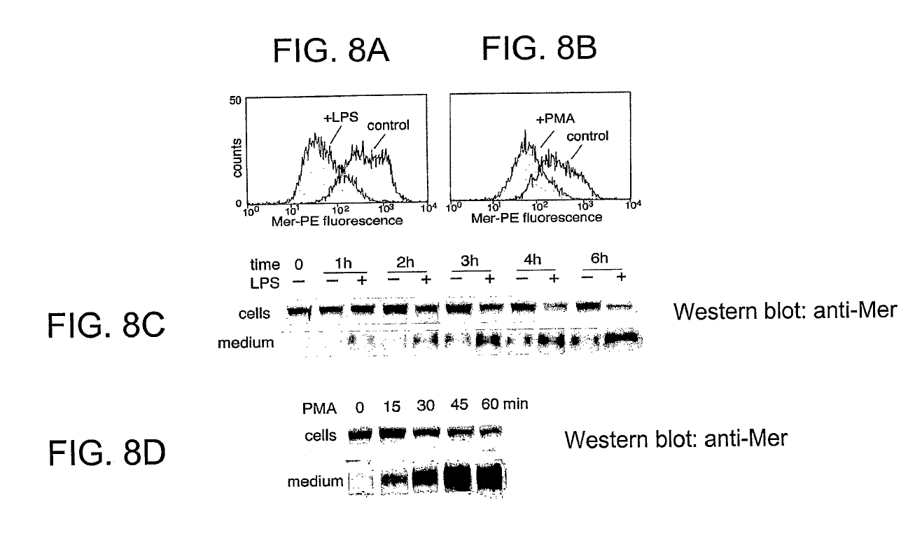

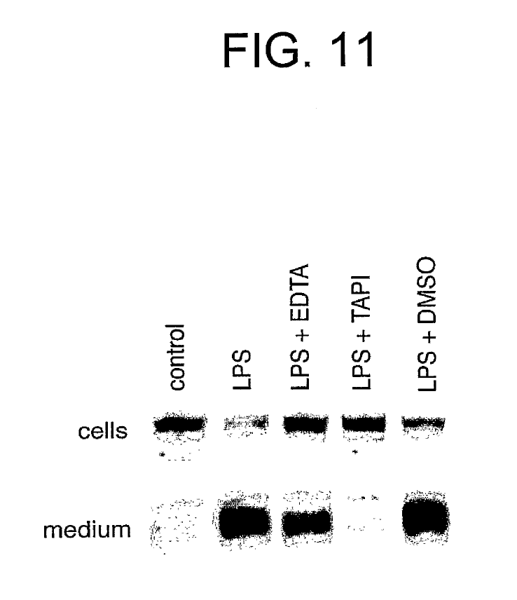

In one embodiment of the invention, the 165-170 kD Mer protein in platelets and the 195-210 kD Mer protein in monocytes is post-translationally processed by cleavage of the extracellular domain via a Tumor Necrosis Factor-α (TNF) converting enzyme (TACE)-like metalloprotease. The cleavage results in a soluble extracellular domain protein (approximately 150 kD) (FIGS. 7, 8, 9 and 10) and a membrane-bound kinase domain. The proteolytic cleavage of Mer can be enhanced by lipopolysaccharide (LPS) and Phorbol 12-myristate B-acetate (PMA) (FIG. 8) and can be specifically inhibited by a TNF-α Protease Inhibitor (TAPI), a TACE inhibitor (FIG. 11). In an alternate embodiment of the invention, a soluble Mer protein is produced as a result of mRNA splicing. Sequence analysis of some full-length Mer samples has led the present inventors to discover the existence of a novel exon encoding a stop codon. This novel RNA splice form consists of exons 1 through 7 in human Mer gene juxtaposed to a novel exon 7A. The resulting truncated, soluble Mer contains 381 amino acids from exons 1 through 7 and 12 amino acids from exon 7A prior to the inframe stop codon.

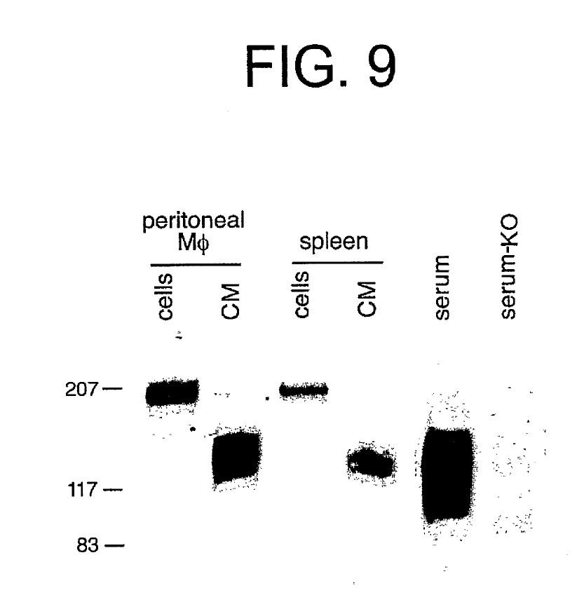

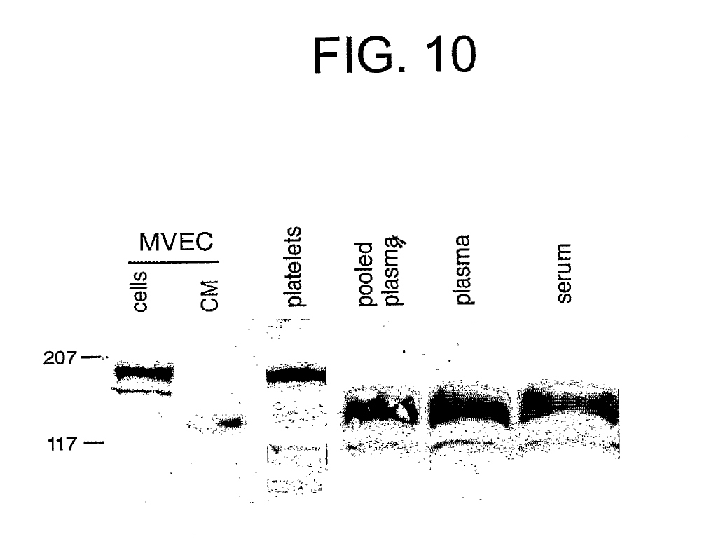

Significant amounts of the soluble Mer protein are present in human and mouse serum (FIGS. 9 and 10). As described in additional detail below, the soluble form can be produced by recombinant methods. Such a recombinant form could be created with alternative forms of glycosylation or indeed, with no glycosylation at all. In a preferred embodiment of the present invention, the soluble Mer protein is produced as a specific glycosylation form that is useful in a diagnostic or therapeutic method of the present invention. The glycosylation forms include any of the forms described herein, including: a fully glycosylated 195-210 kD Mer glycoform; a glycoform having a molecular weight of from about 165 kD to about 170 kD; a glycoform having a molecular weight of from about 170 kD to about 190 kD; a glycoform having a molecular weight of from about 135 kD to about 140 kD, and a glycoform having a molecular weight of from about 190 kD to about 195 kD.

In one embodiment of the present invention, the discovery of different Mer glycoforms by the present inventors is used to selectively and advantageously produce soluble Mer glycoforms that are therapeutically useful because they are competitive inhibitors of a Mer glycoform that is expressed by a particular cell type. For example, a Mer protein having a particular level or pattern of glycosylation may have a higher binding affinity for a Mer ligand (e.g., Gas6 or Protein S) than the endogenous Mer receptor that is expressed by a particular cell type. Such a Mer glycoform can be produced as a soluble Mer protein and then used to inhibit the binding of a Mer ligand to its endogenous Mer receptor, for example, in a therapeutic treatment of a clotting disorder or cancer. Such a soluble Mer glycoform can be targeted to the cell type of interest, if desired.

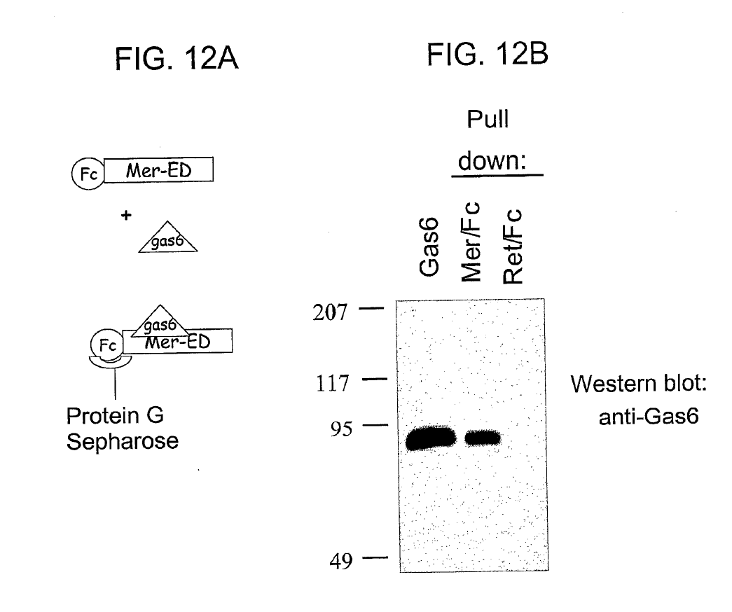

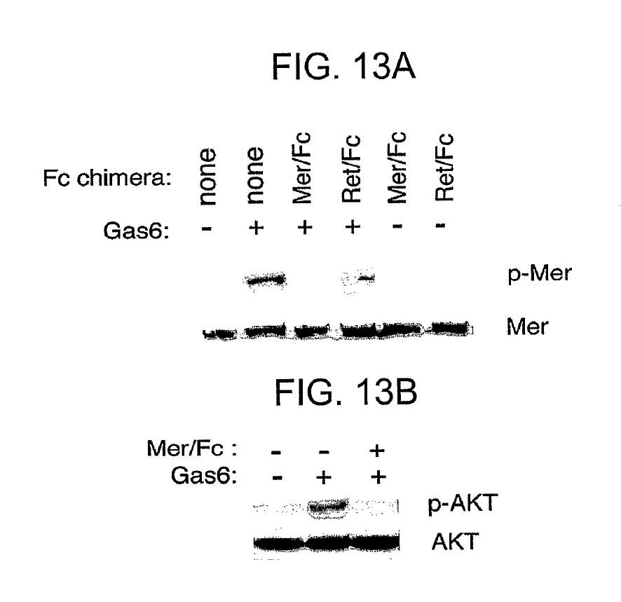

The present inventors have shown that soluble Mer protein directly binds the Mer Gas6 (FIG. 12) and Protein S, thereby inhibiting stimulation of full-length Mer (FIGS. 6 and 13). Gas6 and Protein S are also ligands for Axl and Tyro-3. The cleavage of Mer therefore represents a mechanism of directly regulating (including upregulating or downregulating) the numerous functions of the Mer, Axl and Tyro-3 ligands, including promoting platelet adhesion and clot stability, stimulating cell proliferation, inducing cell adhesion and chemotaxis, and preventing apoptosis. Finally, the cleavage of Mer or the use of its soluble form, and specifically, cleavage of specific Mer glycoforms or the use of soluble forms of the Mer glycoforms described herein, represents a mechanism to indirectly modulate (regulate, modify) the activities of the Mer, Axl and Tyro-3 tyrosine kinases by modulating the functions of Protein S, Gas6 and other Mer ligands.

Accordingly, embodiments of the present invention also pertain to isolated polypeptides described herein, and specifically various Mer glycoforms, and particularly aberrant Mer glycoforms and/or soluble forms of the extracellular Mer receptor tyrosine kinase, including those expressed by nucleic acids encoding a Mer variant (described below).

As used herein, reference to an isolated protein or polypeptide in the present invention, including an isolated Mer protein, includes full-length proteins, fusion proteins, or any fragment or other homologue (variant) of such a protein. The amino acid sequence for Mer from human, mouse, rat and chicken are described herein as exemplary Mer proteins (see above). Reference to a Mer protein can include, but is not limited to, purified Mer protein, recombinantly produced Mer protein, membrane bound Mer protein, Mer protein complexed with lipids, soluble Mer protein, any Mer glycoform, and isolated Mer protein associated with other proteins. More specifically, an isolated protein, such as a Mer protein, according to the present invention, is a protein (including a polypeptide or peptide) that has been removed from its natural milieu (i.e., that has been subject to human manipulation) and can include purified proteins, partially purified proteins, recombinantly produced proteins, and synthetically produced proteins, for example. As such, “isolated” does not reflect the extent to which the protein has been purified. The term “polypeptide” refers to a polymer of amino acids, and not to a specific length; thus, peptides, oligopeptides and proteins are included within the definition of a polypeptide. As used herein, a polypeptide is said to be “purified” when it is substantially free of cellular material when it is isolated from recombinant and non-recombinant cells, or free of chemical precursors or other chemicals when it is chemically synthesized. A polypeptide, however, can be joined to another polypeptide with which it is not normally associated in a cell (e.g., in a “fusion protein”) and still be “isolated” or “purified.”

In addition, and by way of example, a “human Mer protein” refers to a Mer protein (generally including a homologue of a naturally occurring Mer protein) from a human (Homo sapiens) or to a Mer protein that has been otherwise produced from the knowledge of the structure (e.g., sequence) and perhaps the function of a naturally occurring Mer protein from Homo sapiens. In other words, a human Mer protein includes any Mer protein that has substantially similar structure and function of a naturally occurring Mer protein from Homo sapiens or that is a biologically active (i.e., has biological activity) homologue of a naturally occurring Mer protein from Homo sapiens as described in detail herein. As such, a human Mer protein can include purified, partially purified, recombinant, mutated/modified and synthetic proteins. According to the present invention, the terms “modification” and “mutation” can be used interchangeably, particularly with regard to the modifications/mutations to the amino acid sequence of Mer (or nucleic acid sequences) described herein. An isolated protein useful as an antagonist or agonist according to the present invention can be isolated from its natural source, produced recombinantly or produced synthetically.

The polypeptides of the invention also encompass fragment and sequence variants, generally referred to herein as homologues. As used herein, the term “homologue” is used to refer to a protein or peptide which differs from a naturally occurring protein or peptide (i.e., the “prototype” or “wild-type” protein) by minor modifications to the naturally occurring protein or peptide, but which maintains the basic protein and side chain structure of the naturally occurring form. Such changes include, but are not limited to: changes in one or a few amino acid side chains; changes one or a few amino acids, including deletions (e.g., a truncated version of the protein or peptide) insertions and/or substitutions; changes in stereochemistry of one or a few atoms; and/or minor derivatizations, including but not limited to: methylation, glycosylation, phosphorylation, acetylation, myristoylation, prenylation, palmitation, amidation and/or addition of glycosylphosphatidyl inositol. A homologue can have either enhanced, decreased, or substantially similar properties as compared to the naturally occurring protein or peptide. A homologue can include an agonist of a protein or an antagonist of a protein.

Variants or homologues include a substantially homologous polypeptide encoded by the same genetic locus in an organism, i.e., an allelic variant, as well as other splicing variants. A naturally occurring allelic variant of a nucleic acid encoding a protein is a gene that occurs at essentially the same locus (or loci) in the genome as the gene which encodes such protein, but which, due to natural variations caused by, for example, mutation or recombination, has a similar but not identical sequence. Allelic variants typically encode proteins having similar activity to that of the protein encoded by the gene to which they are being compared. One class of allelic variants can encode the same protein but have different nucleic acid sequences due to the degeneracy of the genetic code. Allelic variants can also comprise alterations in the 5′ or 3′ untranslated regions of the gene (e.g., in regulatory control regions). Allelic variants are well known to those skilled in the art.

The terms variant or homologue may also encompass polypeptides derived from other genetic loci in an organism, but having substantial homology to any of the previously defined aberrant Mer glycoforms or a soluble form of the extracellular Mer receptor tyrosine kinase, or polymorphic variants thereof. Variants also include polypeptides substantially homologous or identical to these polypeptides but derived from another organism. Variants also include polypeptides that are substantially homologous or identical to these polypeptides that are produced by chemical synthesis.

Variants also include polypeptides that are substantially homologous or identical to these polypeptides that are produced by recombinant methods. As used herein, two polypeptides (or a region of the polypeptides) are substantially homologous or identical when the amino acid sequences are at least about 45-55%, typically at least about 70-75%, more typically at least about 80-85%, and most typically greater than about 90% or more homologous or identical. In one embodiment, a Mer homologue comprises, consists essentially of, or consists of, an amino acid sequence that is at least about 45%, or at least about 50%, or at least about 55%, or at least about 60%, or at least about 65%, or at least about 70%, or at least about 75%, or at least about 80%, or at least about 85%, or at least about 90%, or at least about 95% identical, or at least about 95% identical, or at least about 96% identical, or at least about 97% identical, or at least about 98% identical, or at least about 99% identical (or any percent identity between 45% and 99%, in whole integer increments), to a naturally occurring Mer amino acid sequence. A homologue of Mer differs from a reference (e.g., wild-type) Mer and therefore is less than 100% identical to the reference Mer at the amino acid level. Wild-type Mer sequences include, but are not limited to, SEQ ID NO:2, SEQ ID NO:4, SEQ ID NO:6 and SEQ ID NO:8.

As used herein, unless otherwise specified, reference to a percent (%) identity refers to an evaluation of homology which is performed using: (1) a BLAST 2.0 Basic BLAST homology search using blastp for amino acid searches and blastn for nucleic acid searches with standard default parameters, wherein the query sequence is filtered for low complexity regions by default (described in Altschul, S. F., Madden, T. L., Schääffer, A. A., Zhang, J., Zhang, Z., Miller, W. & Lipman, D. J. (1997) “Gapped BLAST and PSI-BLAST: a new generation of protein database search programs.” Nucleic Acids Res. 25:3389-3402, incorporated herein by reference in its entirety); (2) a BLAST 2 alignment (using the parameters described below); (3) and/or PSI-BLAST with the standard default parameters (Position-Specific Iterated BLAST. It is noted that due to some differences in the standard parameters between BLAST 2.0 Basic BLAST and BLAST 2, two specific sequences might be recognized as having significant homology using the BLAST 2 program, whereas a search performed in BLAST 2.0 Basic BLAST using one of the sequences as the query sequence may not identify the second sequence in the top matches. In addition, PSI-BLAST provides an automated, easy-to-use version of a “profile” search, which is a sensitive way to look for sequence homologues. The program first performs a gapped BLAST database search. The PSI-BLAST program uses the information from any significant alignments returned to construct a position-specific score matrix, which replaces the query sequence for the next round of database searching. Therefore, it is to be understood that percent identity can be determined by using any one of these programs.

Two specific sequences can be aligned to one another using BLAST 2 sequence as described in Tatusova and Madden, (1999), “Blast 2 sequences—a new tool for comparing protein and nucleotide sequences”, FEMS Microbiol Lett. 174:247-250, incorporated herein by reference in its entirety. BLAST 2 sequence alignment is performed in blastp or blastn using the BLAST 2.0 algorithm to perform a Gapped BLAST search (BLAST 2.0) between the two sequences allowing for the introduction of gaps (deletions and insertions) in the resulting alignment. For purposes of clarity herein, a BLAST 2 sequence alignment is performed using the standard default parameters as follows.

For blastn, using 0 BLOSUM62 matrix:

Reward for match=1

Penalty for mismatch=−2

Open gap (5) and extension gap (2) penalties

gap x_dropoff (50) expect (10) word size (11) filter (on)

For blastp, using 0 BLOSUM62 matrix:

Open gap (11) and extension gap (1) penalties

gap x_dropoff (50) expect (10) word size (3) filter (on).

Embodiments of the invention also encompass polypeptides having a lower degree of identity but having sufficient similarity so as to perform one or more of the same functions performed by a polypeptide of the invention. A variant polypeptide can differ in amino acid sequence by one or more substitutions, deletions, insertions, inversions, fusions, and truncations or a combination of any of these. Further, variant polypeptides can be fully functional or can lack function in one or more activities.

Embodiments of the invention also include polypeptide fragments of the polypeptides of the invention. The invention also encompasses fragments of the variants of the polypeptides described herein. As used herein, a fragment comprises at least 6 contiguous amino acids and includes any fragment of a full-length Mer protein described herein, including the entire extracellular domain of Mer or any portion thereof that retains the ability to bind to a Mer ligand. Useful fragments include those that retain one or more of the biological activities of the polypeptide (e.g., ligand binding and/or signal transduction capability) as well as fragments that can be used as an immunogen to generate polypeptide-specific antibodies. Fragments can be discrete (not fused to other amino acids or polypeptides) or can be within a larger polypeptide. Further, several fragments can be comprised within a single larger polypeptide. Therefore, fragments can include any size fragment between about 6 amino acids and 998 amino acids, including any fragment in between, in whole integer increments (e.g., 7, 8, 9 . . . 67, 68, 69 . . . 278, 279, 280 amino acids).

Embodiments of the invention thus provide chimeric or fusion polypeptides. These comprise a polypeptide of the invention operatively linked to a heterologous protein or polypeptide having an amino acid sequence not substantially homologous to the polypeptide. “Operatively linked” indicates that the polypeptide and the heterologous protein (also called a fusion segment or fusion partner) are fused in-frame. The heterologous protein can be fused to the N-terminus or C-terminus of the polypeptide. A chimeric or fusion polypeptide can be produced by standard recombinant DNA techniques well known in the art. Preferred heterologous proteins according to the present invention include, but are not limited to, any proteins or peptides that can: enhance a protein's stability; provide other desirable biological activity; and/or assist with the purification of a protein (e.g., by affinity chromatography), or provide another protein function (e.g., as in a chimeric protein). A suitable heterologous protein can be a domain of any size that has the desired function (e.g., imparts increased stability, solubility, action or biological activity; simplifies purification of a protein; or provides the additional protein function). In one embodiment, a suitable heterologous protein with which a chimeric or fusion protein can be produced is an antibody fragment and particularly, the Fc portion of an immunoglobulin protein. Any fusion or chimera partner that enhances the stability or half-life of Mer in vivo, for example, is contemplated for use in the present invention.

As used herein, the phrase “Mer agonist” refers to any compound that is characterized by the ability to agonize (e.g., stimulate, induce, increase, enhance, or mimic) the biological activity of a naturally occurring Mer as described herein, and includes any Mer homologue, binding protein (e.g., an antibody), agent that interacts with Mer or mimics Mer, or any suitable product of drug/compound/peptide design or selection which is characterized by its ability to agonize (e.g., stimulate, induce, increase, enhance) the biological activity of a naturally occurring Mer protein in a manner similar to the natural agonist, Mer.

Similarly, the phrase, “Mer antagonist” refers to any compound which inhibits (e.g., antagonizes, reduces, decreases, blocks, reverses, or alters) the effect of an Mer agonist as described above. More particularly, a Mer antagonist is capable of acting in a manner relative to Mer activity, such that the biological activity of the natural agonist Mer, is decreased in a manner that is antagonistic (e.g., against, a reversal of, contrary to) to the natural action of Mer. Such antagonists can include, but are not limited to, a protein (e.g., soluble Mer), peptide, or nucleic acid (including ribozymes, RNAi, aptamers, and antisense), antibodies and antigen binding fragments thereof, or product of drug/compound/peptide design or selection that provides the antagonistic effect.

Homologues of Mer, including peptide and non-peptide agonists and antagonists of Mer (analogues), can be products of drug design or selection and can be produced using various methods known in the art. Such homologues can be referred to as mimetics. A mimetic refers to any peptide or non-peptide compound that is able to mimic the biological action of a naturally occurring peptide, often because the mimetic has a basic structure that mimics the basic structure of the naturally occurring peptide and/or has the salient biological properties of the naturally occurring peptide. Mimetics can include, but are not limited to: peptides that have substantial modifications from the prototype such as no side chain similarity with the naturally occurring peptide (such modifications, for example, may decrease its susceptibility to degradation); anti-idiotypic and/or catalytic antibodies, or fragments thereof; non-proteinaceous portions of an isolated protein (e.g., carbohydrate structures); or synthetic or natural organic molecules, including nucleic acids and drugs identified through combinatorial chemistry, for example. Such mimetics can be designed, selected and/or otherwise identified using a variety of methods known in the art. Various methods of drug design, useful to design or select mimetics or other therapeutic compounds useful in the present invention are disclosed in Maulik et al., 1997, Molecular Biotechnology: Therapeutic Applications and Strategies, Wiley-Liss, Inc., which is incorporated herein by reference in its entirety.

Homologues can be produced using techniques known in the art for the production of proteins including, but not limited to, direct modifications to the isolated, naturally occurring protein, direct protein synthesis, or modifications to the nucleic acid sequence encoding the protein using, for example, classic or recombinant DNA techniques to effect random or targeted mutagenesis. For smaller peptides, chemical synthesis methods may be preferred. For example, such methods include well known chemical procedures, such as solution or solid-phase peptide synthesis, or semi-synthesis in solution beginning with protein fragments coupled through conventional solution methods. Such methods are well known in the art and may be found in general texts and articles in the area such as: Merrifield, 1997, Methods Enzymol. 289:3-13; Wade et al., 1993, Australas Biotechnol. 3(6):332-336; Wong et al., 1991, Experientia 47(11-12):1123-1129; Carey et al., 1991, Ciba Found Symp. 158:187-203; Plaue et al., 1990, Biologicals 18(3):147-157; Bodanszky, 1985, Int. J. Pept. Protein Res. 25(5):449-474; or H. Dugas and C. Penney, BIOORGANIC CHEMISTRY, (1981) at pages 54-92, all of which are incorporated herein by reference in their entirety. For example, peptides may be synthesized by solid-phase methodology utilizing a commercially available peptide synthesizer and synthesis cycles supplied by the manufacturer. One skilled in the art recognizes that the solid phase synthesis could also be accomplished using the FMOC strategy and a TFA/scavenger cleavage mixture.

The polypeptides of the invention can be purified to homogeneity. It is understood, however, that preparations in which the polypeptide is not purified to homogeneity are useful. The critical feature is that the preparation allows for the desired function of the polypeptide, even in the presence of considerable amounts of other components. Indeed, in some of the assay formats contemplated by the invention, the natural biological milieu may contain important other proteins that affect the normal activity of Mer and the soluble forms of Mer. Thus, the invention encompasses various degrees of purity. In one embodiment, the language “substantially free of cellular material” includes preparations of the polypeptide having less than about 30% (by dry weight) other proteins (i.e., contaminating protein), less than about 20% other proteins, less than about 10% other proteins, or less than about 5% other proteins.

In one aspect of the present invention, the soluble form of the Mer receptor tyrosine kinase is a product of TACE-like metalloprotease cleavage near the transmembrane domain of the full-length Mer receptor that removes the intracellular kinase of Mer as well as much, if not all of the transmembrane domain of the protein.

In a preferred aspect of the present invention, Mer proteins, and especially soluble Mer proteins, are produced as a specific glycoform of Mer, and in one aspect, as a specific glycoform described herein, including a glycoform that is associated with a particular cell type or a glycoform that is selected to compete with a natural Mer receptor for binding to a Mer ligand. Glycosylation is a post-translational modification. Glycosylation of Mer proteins of the invention can be achieved by any suitable method. For example, a Mer glycoform corresponding to a particular cell type can be produced by recombinantly expressing the Mer protein (including soluble Mer proteins) in a host cell of the cell type that naturally produces the specified Mer glycoform. Alternatively, Mer homologues can be produced (e.g., using recombinant technology) that have altered (mutated, modified) glycosylation sites, such that the desired glycosylation pattern and/or level is achieved. Glycosylation of a protein can also be achieved by glycosylating an isolated protein in vitro, after its production and isolation or secretion from a cell.

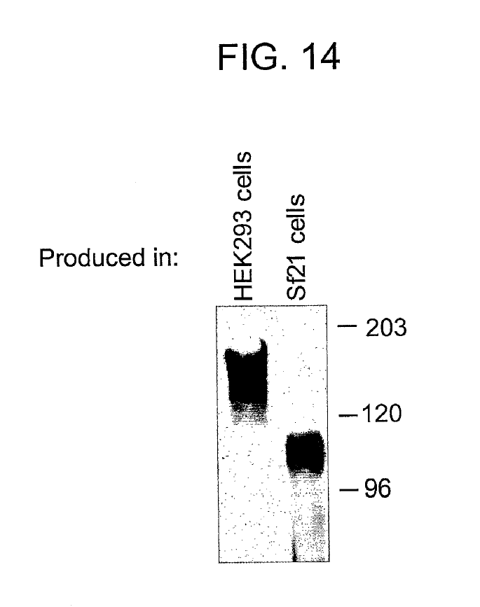

For example, preferred Mer proteins include Mer proteins that are wild-type Mer proteins that have been post-translationally glycosylated by a particular cell-type to provide a specific Mer glycoform. Preferred Mer proteins also include Mer protein variants (homologues) with glycosylation sites that differ from the wild-type protein such that Mer proteins that are less than or more than fully glycosylated as compared to a wild-type protein produced by monocytic cells are produced. Desirable Mer glycoforms to produce according to the invention, as discussed above, include, but are not limited to, Mer glycoforms having a molecular weight (due to altered glycosylation), of from about 195 to 210 kD, from about 165 kD to about 170 kD, less than about 160 kD, or between about 170 kD to about 195 kD, such as those glycoforms found in leukemia and lymphoma cells. Particularly desirable glycoforms to produce by modifying glycosylation sites in the protein include, but are not limited to, (1) a glycoform from about 165 kD to about 170 kD; (2) a glycoform from about 170 kD to about 190 kD; (3) a glycoform from about 135 kD to about 140 kD; and (4) a glycoform from about 190 kD to about 195 kD. As discussed above, such glycoforms could be the result of mutations in the nucleic acid molecule resulting in reduced or altered glycosylation sites on the Mer receptor tyrosine kinase, mutations in other cellular proteins which result in faulty post-translational processing, mutations which produce truncated or variant extracellular domains, or the cell-type in which the Mer protein is expressed and post-translationally modified. For example, aberrantly glycosylated forms of the Mer receptor tyrosine kinase can be produced by expressing the protein in any of a variety of mammalian cell types known to produce varying glycosylation patterns, including the Jurkat human leukemia, U937 human monocyte, K562 human chronic myelogenous, and the HEK293 human kidney cell lines (FIG. 14). For example, using the HEK293 cell line, a specific sMer glycoform has been produced (FIG. 7C).

According to the present invention, an isolated Mer protein, including a biologically active homologue or fragment thereof, has at least one characteristic of biological activity of activity a wild-type, or naturally occurring Mer protein (which can vary depending on whether the homologue or fragment is an agonist, antagonist, or mimic of Mer, and the isoform of Mer). Biological activity of Mer and methods of determining the same have been described previously herein.

In one embodiment, a particularly preferred Mer protein is a Mer protein variant and/or a Mer glycoform that preferentially binds to one Mer ligand as compared to another Mer ligand. For example, known Mer ligands include Gas6 and Protein S. In one aspect of the invention, the Mer protein, preferably a soluble Mer protein, is either a variant of Mer or a glycoform of Mer, or both, that binds to Protein S with a statistically significantly higher binding affinity than the binding of the Mer protein to Gas6. In another aspect, the Mer protein, preferably a soluble Mer protein, is either a variant of Mer or a glycoform of Mer, or both, that binds to Gas6 with a statistically significantly higher binding affinity than the binding of the Mer protein to Protein S.

A particularly preferred Mer protein of the invention includes any soluble Mer protein and preferably any soluble form of any of the Mer glycoforms described herein, and most preferably, any soluble form of any of the aberrant glycosylated Mer proteins described herein, or any soluble form of a Mer protein that is selectively glycosylated, such as to provide a competitive inhibitor that preferentially binds to Mer ligand as compared to an endogenous Mer cellular receptor. In one embodiment of the present invention, a preferred soluble Mer protein is a Mer chimeric protein consisting of the Mer extracellular domain (e.g., positions 1 to about 473 of SEQ ID NO:2), or a smaller portions of this extracellular domain that retain the ability to bind to at least one Mer ligand, fused to the Fc region of human IgG and expressed in different mammalian cell lines, such as HEK293, to yield specific glycoslyation forms of soluble Mer (FIG. 14). Other glycoforms of soluble Mer can be made in a similar manner by expressing the Mer-Fc construct in other mammalian cell lines since cell types glycosylate Mer in a distinct manner (FIG. 7C). Without being bound by theory, the present inventors believe that the different glycoforms of soluble Mer are believed to have different binding affinities to different Mer ligands, including Protein S and Gas6. The Mer extracellular domain can include any extracellular fragment of Mer that binds to a Mer ligand. Such a soluble Mer protein is glycosylated to form a fully glycosylated sMer, or any of the less-glycosylated Mer proteins as described herein.

Nucleic Acid Molecules Encoding sMer and Mer Variants

Another embodiment of the invention relates to an isolated nucleic acid molecule, or complement thereof, encoding a Mer protein (full-length wild-type) or any homologue thereof (including variants and fragments), and can include Mer proteins in which the resulting amino acid sequence of the encoded Mer protein is altered (e.g., modified by substitution, deletion and/or insertion) to add or eliminate those amino acids that form glycosylation sites in the encoded protein, such that aberrantly glycosylated Mer glycoforms can be produced. In particular, one aspect of the present invention relates to nucleic acid molecules encoding Mer proteins with glycosylation sites that differ from the wild-type protein, so that Mer glycoforms including, but not limited to, Mer glycoforms having a molecular weight (due to altered glycosylation) of less than about 160 kD or between about 170 kD to about 195 kD, such as those glycoforms found in leukemia and lymphoma cells. Particularly desirable glycoforms to produce by modifying glycosylation sites in the protein include, but are not limited to, (1) a glycoform from about 165 kD to about 170 kD; (2) a glycoform from about 170 kD to about 190 kD; (3) a glycoform from about 135 kD to about 140 kD; and (4) a glycoform from about 190 kD to about 195 kD. Such glycoforms produced by nucleic acid molecules of the invention could be the result of mutations in the nucleic acid molecule resulting in reduced or altered glycosylation sites on the Mer receptor tyrosine kinase, including mutations which produce truncated or variant extracellular domains. Such mutations resulting in aberrant glycoforms can be introduced into the molecule using standard molecular biology techniques such as site directed mutagenesis. Site directed mutagenesis could be targeted against 13 potential NH₂ linked glycoslyation sites in the Mer extracellular domain, which are identified by the amino acid sequence: NXS/T.

The present invention also relates to an isolated nucleic acid molecule, or complement thereof, encoding a soluble form of the extracellular Mer receptor tyrosine kinase as described previously. This sMer can also be engineered to have glycosylation patterns identical to the aberrant forms using techniques as described above. As discussed above, it is one aspect of the present invention to provide particular glycoforms of the sMer that have different binding affinities for Mer ligands, including Protein S and Gas6 and indeed, without being bound by theory, the present inventors believe that different glycoforms of Mer have different ligand binding affinities. Assays for measuring binding affinities are well-known in the art. In one embodiment, a BIAcore machine can be used to determine the binding constant of a complex between the target protein (e.g., a Mer glycoform) and a natural ligand. For example, the Mer glycoform can be immobilized on a substrate. A natural or synthetic ligand is contacted with the substrate to form a complex. The dissociation constant for the complex can be determined by monitoring changes in the refractive index with respect to time as buffer is passed over the chip (O'Shannessy et al. Anal. Biochem. 212:457-468 (1993); Schuster et al., Nature 365:343-347 (1993)). Contacting a second compound (e.g., a different ligand or a different soluble Mer glycoform) at various concentrations at the same time as the first ligand and monitoring the response function (e.g., the change in the refractive index with respect to time) allows the complex dissociation constant to be determined in the presence of the second compound and indicates whether the second compound is an inhibitor of the complex. Other suitable assays for measuring the binding of a soluble receptor to a ligand include, but are not limited to, Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microarray, microscopy, fluorescence activated cell sorting (FACS), and flow cytometry.

The isolated nucleic acid molecules of the present invention can be RNA, for example, mRNA, or DNA, such as cDNA and genomic DNA. DNA molecules can be double-stranded or single-stranded; single stranded RNA or DNA can be either the coding, or sense, strand or the non-coding, or antisense, strand. The nucleic acid molecule can include all or a portion of the coding sequence of the gene and can further comprise additional non-coding sequences such as introns and non-coding 3′ and 5′ sequences (including regulatory sequences, for example). The nucleic acid can also comprise the sequences that would code for the aberrantly glycosylated glycoforms. Additionally, the nucleic acid molecule can be fused to a marker sequence, for example, a sequence that encodes a polypeptide to assist in isolation or purification of the polypeptide.

An “isolated” nucleic acid molecule, as used herein, is one that is separated from nucleic acids that normally flank the gene or nucleotide sequence (as in genomic sequences) and/or has been completely or partially purified from other transcribed sequences (e.g., as in an RNA library). For example, an isolated nucleic acid of the invention may be substantially isolated with respect to the complex cellular milieu in which it naturally occurs, or culture medium when produced by recombinant techniques, or chemical precursors or other chemicals when chemically synthesized. In some instances, the isolated material will form part of a composition (for example, a crude extract containing other substances), buffer system or reagent mix. In other circumstances, the material may be purified to essential homogeneity, for example as determined by PAGE or column chromatography such as HPLC.

The nucleic acid molecule can be fused to other coding or regulatory sequences and still be considered isolated. Thus, recombinant DNA contained in a vector is included in the definition of “isolated” as used herein. Also, isolated nucleic acid molecules include recombinant DNA molecules in heterologous host cells, as well as partially or substantially purified DNA molecules in solution. “Isolated” nucleic acid molecules also encompass in vivo and in vitro RNA transcripts of the DNA molecules of the present invention. An isolated nucleic acid molecule or nucleotide sequence can include a nucleic acid molecule or nucleotide sequence that is synthesized chemically or by recombinant means. Therefore, recombinant DNA contained in a vector is included in the definition of “isolated” as used herein. Also, isolated nucleotide sequences include partially or substantially purified DNA molecules in solution. In vivo and in vitro RNA transcripts of the DNA molecules of the present invention are also encompassed by “isolated” nucleotide sequences. Such isolated nucleotide sequences are useful in the manufacture of the encoded polypeptide, as probes for isolating homologous sequences (e.g., from other mammalian species), for gene mapping (e.g., by in situ hybridization with chromosomes), or for detecting expression of the gene in tissue (e.g., human tissue), such as by Northern blot analysis.

The present invention also pertains to variant nucleic acid molecules that are not necessarily found in nature but which encode novel aberrant Mer glycoforms (e.g., by altering one or more glycosylation sites on the encoded protein) and/or a soluble form of the extracellular Mer receptor tyrosine kinase. Thus, for example, DNA molecules which comprise a sequence that is different from the naturally-occurring nucleotide sequence but which codes for aberrant Mer glycoforms or a soluble form of the extracellular Mer receptor tyrosine kinase polypeptide of the present invention are also the subject of this invention. The invention also encompasses nucleotide sequences encoding portions (fragments), or encoding variant polypeptides such as analogues or derivatives of novel Mer glycoforms or a soluble form of the Mer receptor tyrosine kinase. Such variants can be naturally occurring, such as in the case of allelic variation or single nucleotide polymorphisms, or non-naturally-occurring, such as those induced by various mutagens and mutagenic processes. Intended variations include, but are not limited to, addition, deletion and substitution of one or more nucleotides that can result in conservative or non-conservative amino acid changes, including additions and deletions.

Other alterations of the nucleic acid molecules of the invention can include, for example, labeling, methylation, internucleotide modifications such as uncharged linkages (e.g., methyl phosphonates, phosphotriesters, phosphoamidates, carbamates), charged linkages (e.g., phosphorothioates, phosphorodithioates), pendent moieties (e.g., polypeptides), intercalators (e.g., acridine, psoralen), chelators, alkylators, and modified linkages (e.g., alpha anomeric nucleic acids). Also included are synthetic molecules that mimic nucleic acid molecules in the ability to bind to designated sequences via hydrogen bonding and other chemical interactions. Such molecules include, for example, those in which peptide linkages substitute for phosphate linkages in the backbone of the molecule.

The invention also pertains to nucleic acid molecules that hybridize under high stringency hybridization conditions, such as for selective hybridization, to a nucleotide sequence described herein (e.g., nucleic acid molecules which specifically hybridize to a nucleotide sequence encoding polypeptides described herein, and, optionally, have an activity of the polypeptide). In one embodiment, the invention includes variants described herein which hybridize under high stringency hybridization conditions (e.g., for selective hybridization) to a nucleotide sequence encoding novel Mer glycoforms (e.g., where one or more glycosylation sites on the protein is disrupted), including novel aberrant Mer glycoforms described herein, and/or a soluble form of the extracellular Mer receptor tyrosine kinase or the complements thereof.

The present invention also provides isolated nucleic acid molecules that contain a fragment or portion that hybridizes under highly stringent conditions to a nucleic acid encoding an aberrant Mer glycoform or a soluble form of the extracellular Mer receptor tyrosine kinase or the complements thereof “Stringency conditions” for hybridization is a term of art which refers to the incubation and wash conditions, e.g., conditions of temperature and buffer concentration, which permit hybridization of a particular nucleic acid to a second nucleic acid; the first nucleic acid may be perfectly (i.e., 100%) complementary to the second, or the first and second may share some degree of complementarity which is less than perfect (e.g., 70%, 75%, 85%, 95%). For example, certain high stringency conditions can be used which distinguish perfectly complementary nucleic acids from those of less complementarity. “High stringency conditions”, “moderate stringency conditions” and “low stringency conditions” for nucleic acid hybridizations are explained on pages 2.10.1-2.10.16 and pages 6.3.1-6.3.6 in Current Protocols in Molecular Biology (Ausubel, F. M. et al., “Current Protocols in Molecular Biology”, John Wiley & Sons, (1998), the entire teachings of which are incorporated by reference herein). Typically, conditions are used such that sequences at least about 60%, at least about 70%, at least about 80%, at least about 90% or at least about 95% or more identical to each other remain hybridized to one another. By varying hybridization conditions from a level of stringency at which no hybridization occurs to a level at which hybridization is first observed, conditions which will allow a given sequence to hybridize (e.g., selectively) with the most similar sequences in the sample can be determined.

More particularly, moderate stringency hybridization and washing conditions, as referred to herein, refer to conditions which permit isolation of nucleic acid molecules having at least about 70% nucleic acid sequence identity with the nucleic acid molecule being used to probe in the hybridization reaction (i.e., conditions permitting about 30% or less mismatch of nucleotides). High stringency hybridization and washing conditions, as referred to herein, refer to conditions which permit isolation of nucleic acid molecules having at least about 80% nucleic acid sequence identity with the nucleic acid molecule being used to probe in the hybridization reaction (i.e., conditions permitting about 20% or less mismatch of nucleotides). Very high stringency hybridization and washing conditions, as referred to herein, refer to conditions which permit isolation of nucleic acid molecules having at least about 90% nucleic acid sequence identity with the nucleic acid molecule being used to probe in the hybridization reaction (i.e., conditions permitting about 10% or less mismatch of nucleotides). As discussed above, one of skill in the art can use the formulae in Meinkoth et al., ibid. to calculate the appropriate hybridization and wash conditions to achieve these particular levels of nucleotide mismatch. Such conditions will vary, depending on whether DNA:RNA or DNA:DNA hybrids are being formed. Calculated melting temperatures for DNA:DNA hybrids are 10° C. less than for DNA:RNA hybrids. In particular embodiments, stringent hybridization conditions for DNA:DNA hybrids include hybridization at an ionic strength of 6×SSC (0.9 M Na⁺) at a temperature of between about 20° C. and about 35° C. (lower stringency), more preferably, between about 28° C. and about 40° C. (more stringent), and even more preferably, between about 35° C. and about 45° C. (even more stringent), with appropriate wash conditions. In particular embodiments, stringent hybridization conditions for DNA:RNA hybrids include hybridization at an ionic strength of 6×SSC (0.9 M Na⁺) at a temperature of between about 30° C. and about 45° C., more preferably, between about 38° C. and about 50° C., and even more preferably, between about 45° C. and about 55° C., with similarly stringent wash conditions. These values are based on calculations of a melting temperature for molecules larger than about 100 nucleotides, 0% formamide and a G+C content of about 40%. Alternatively, Tₘ can be calculated empirically as set forth in Sambrook et al., supra, pages 9.31 to 9.62. In general, the wash conditions should be as stringent as possible, and should be appropriate for the chosen hybridization conditions. For example, hybridization conditions can include a combination of salt and temperature conditions that are approximately 20-25° C. below the calculated Tₘ of a particular hybrid, and wash conditions typically include a combination of salt and temperature conditions that are approximately 12-20° C. below the calculated Tₘ of the particular hybrid. One example of hybridization conditions suitable for use with DNA:DNA hybrids includes a 2-24 hour hybridization in 6×SSC (50% formamide) at about 42° C., followed by washing steps that include one or more washes at room temperature in about 2×SSC, followed by additional washes at higher temperatures and lower ionic strength (e.g., at least one wash as about 37° C. in about 0.1×-0.5×SSC, followed by at least one wash at about 68° C. in about 0.1×-0.5×SSC).

In a related aspect of the invention, the nucleic acid fragments of the invention are used as probes or primers in assays such as those described herein. “Probes” or “primers” are oligonucleotides that hybridize in a base-specific manner to a complementary strand of nucleic acid molecules. By “base specific manner” is meant that the two sequences must have a degree of nucleotide complementarity sufficient for the primer or probe to hybridize. Accordingly, the primer or probe sequence is not required to be perfectly complementary to the sequence of the template. Non-complementary bases or modified bases can be interspersed into the primer or probe, provided that base substitutions do not substantially inhibit hybridization. The nucleic acid template may also include “non-specific priming sequences” or “nonspecific sequences” to which the primer or probe has varying degrees of complementarity. Such probes and primers include polypeptide nucleic acids, as described in Nielsen et al., Science, 254, 1497-1500 (1991). Typically, a probe or primer comprises a region of nucleotide sequence that hybridizes to at least about 15, typically about 20-25, and more typically about 40, 50, 75, 100, 150, 200, or more, consecutive nucleotides of a nucleic acid molecule comprising a nucleotide sequence encoding a Mer protein, including an aberrant Mer glycoform (e.g., a Mer glycoform in which glycosylation sites have been modified) or a soluble form of the extracellular Mer receptor tyrosine kinase or the complements thereof.

The nucleic acid molecules of the invention such as those described above can be identified and isolated using standard molecular biology techniques and the sequence information provided herein. For example, nucleic acid molecules can be amplified and isolated by the polymerase chain reaction using synthetic oligonucleotide primers designed based on a nucleotide sequence encoding an aberrant Mer glycoform or a soluble form of the extracellular Mer receptor tyrosine kinase or the complements thereof. See generally PCR Technology: Principles and Applications for DNA Amplification (ed. H. A. Erlich, Freeman Press, NY, N.Y., 1992); PCR Protocols. A Guide to Methods and Applications (Eds. Innis, et al., Academic Press, San Diego, Calif., 1990); Mattila et al., Nucleic Acids Res., 19:4967 (1991); Eckert et al., PCR Methods and Applications, 1:17 (1991); PCR (eds. McPherson et al., IRL Press, Oxford); and U.S. Pat. No. 4,683,202. The nucleic acid molecules can be amplified using cDNA, mRNA or genomic DNA as a template, cloned into an appropriate vector and characterized by DNA sequence analysis. Soluble forms of Mer consisting essentially of the extracellular form of Mer and lacking the kinase region of the molecules can be discerned from an inspection of U.S. Pat. No. 5,585,689 taken with the instant disclosure.

Other suitable amplification methods include the ligase chain reaction (LCR) (see Wu and Wallace, Genomics, 4:560 (1989), Landegren et al., Science, 241:1077 (1988)), transcription amplification (Kwoh et al., Proc. Natl. Acad. Sci. USA, 86:1173 (1989)), and self-sustained sequence replication (Guatelli et al., Proc. Nat. Acad. Sci. USA, 87:1874 (1990)) and nucleic acid based sequence amplification (NASBA).

The amplified DNA can be labeled (e.g., with radiolabel or other reporter molecule) and used as a probe for screening a cDNA library derived from human cells, mRNA in zap express, ZIPLOX or other suitable vector. Corresponding clones can be isolated, DNA can obtained following in vivo excision, and the cloned insert can be sequenced in either or both orientations by art recognized methods to identify the correct reading frame encoding a polypeptide of the appropriate molecular weight. For example, the direct analysis of the nucleotide sequence of nucleic acid molecules of the present invention can be accomplished using well-known methods that are commercially available. See, for example, Sambrook et al., Molecular Cloning, A Laboratory Manual (2nd Ed., CSHP, New York 1989); Zyskind et al., Recombinant DNA Laboratory Manual, (Acad. Press, 1988). Using these or similar methods, the polypeptide and the DNA encoding the polypeptide can be isolated, sequenced and further characterized.

In general, the isolated nucleic acid sequences of the invention can be used as molecular weight markers on Southern gels, and as chromosome markers that are labeled to map related gene positions. The nucleic acid sequences can also be used to compare with endogenous DNA sequences in patients to identify genetic disorders and as probes, such as to hybridize and discover related DNA sequences or to subtract out known sequences from a sample. The nucleic acid sequences can further be used to derive primers for genetic fingerprinting, to raise anti-polypeptide antibodies using DNA immunization techniques, and as an antigen to raise anti-DNA antibodies or elicit immune responses. Additionally, and preferably, the nucleotide sequences of the invention can be used to identify and express recombinant polypeptides for analysis, for characterization, for diagnostic use, for therapeutic use, or as markers for tissues in which the corresponding polypeptide is expressed, either constitutively, during tissue differentiation, or in diseased states. The nucleic acid sequences can additionally be used as reagents in the screening and/or diagnostic assays described herein, and can also be included as components of kits (e.g., reagent kits) for use in the screening and/or diagnostic assays described herein.

Such nucleic acid sequences can be incorporated into host cells and expression vectors that are well known in the art. According to the present invention, a recombinant nucleic acid molecule includes at least one isolated nucleic acid molecule of the present invention that is linked to a heterologous nucleic acid sequence. Such a heterologous nucleic acid sequence is typically a recombinant nucleic acid vector (e.g., a recombinant vector) which is suitable for cloning, sequencing, and/or otherwise manipulating the nucleic acid molecule, such as by expressing and/or delivering the nucleic acid molecule into a host cell to form a recombinant cell. Such a vector contains heterologous nucleic acid sequences, that is nucleic acid sequences that are not naturally found adjacent to nucleic acid molecules of the present invention, although the vector can also contain regulatory nucleic acid sequences (e.g., promoters, untranslated regions) which are naturally found adjacent to nucleic acid molecules of the present invention. The vector can be either RNA or DNA, either prokaryotic or eukaryotic, and typically is a virus or a plasmid. The vector can be maintained as an extrachromosomal element (e.g., a plasmid) or it can be integrated into the chromosome. The entire vector can remain in place within a host cell, or under certain conditions, the plasmid DNA can be deleted, leaving behind the nucleic acid molecule of the present invention. The integrated nucleic acid molecule can be under chromosomal promoter control, under native or plasmid promoter control, or under a combination of several promoter controls. Single or multiple copies of the nucleic acid molecule can be integrated into the chromosome. As used herein, the phrase “recombinant nucleic acid molecule” is used primarily to refer to a recombinant vector into which has been ligated the nucleic acid sequence to be cloned, manipulated, transformed into the host cell (i.e., the insert).

The nucleic acid sequence encoding the protein to be produced is inserted into the vector in a manner that operatively links the nucleic acid sequence to regulatory sequences in the vector (e.g., expression control sequences) which enable the transcription and translation of the nucleic acid sequence when the recombinant molecule is introduced into a host cell. According to the present invention, the phrase “operatively linked” refers to linking a nucleic acid molecule to an expression control sequence (e.g., a transcription control sequence and/or a translation control sequence) in a manner such that the molecule can be expressed when transfected (i.e., transformed, transduced, transfected, conjugated or conduced) into a host cell. Transcription control sequences are sequences that control the initiation, elongation, or termination of transcription. Particularly important transcription control sequences are those that control transcription initiation, such as promoter, enhancer, operator and repressor sequences. Suitable transcription control sequences include any transcription control sequence that can function in a host cell into which the recombinant nucleic acid molecule is to be introduced.

Recombinant molecules of the present invention, which can be either DNA or RNA, can also contain additional regulatory sequences, such as translation regulatory sequences, origins of replication, and other regulatory sequences that are compatible with the recombinant cell. In one embodiment, a recombinant molecule of the present invention, including those which are integrated into the host cell chromosome, also contains secretory signals (i.e., signal segment nucleic acid sequences) to enable an expressed protein to be secreted from the cell that produces the protein. Suitable signal segments include a signal segment that is naturally associated with a protein of the present invention or any heterologous signal segment capable of directing the secretion of a protein according to the present invention.

One or more recombinant molecules of the present invention can be used to produce an encoded product of the present invention. In one embodiment, an encoded product is produced by expressing a nucleic acid molecule as described herein under conditions effective to produce the protein. A preferred method to produce an encoded protein is by transfecting a host cell with one or more recombinant molecules to form a recombinant cell. Suitable host cells to transfect include, but are not limited to, any bacterial, fungal (e.g., yeast), insect, plant or animal cell that can be transfected. Host cells can be either untransfected cells or cells that are already transfected with at least one nucleic acid molecule.

According to the present invention, the term “transfection” is used to refer to any method by which an exogenous nucleic acid molecule (i.e., a recombinant nucleic acid molecule) can be inserted into the cell. The term “transformation” can be used interchangeably with the term “transfection” when such term is used to refer to the introduction of nucleic acid molecules into microbial cells, such as bacteria and yeast. In microbial systems, the term “transformation” is used to describe an inherited change due to the acquisition of exogenous nucleic acids by the microorganism and is essentially synonymous with the term “transfection”. However, in animal cells, transformation has acquired a second meaning which can refer to changes in the growth properties of cells in culture after they become cancerous, for example. Therefore, to avoid confusion, the term “transfection” is preferably used with regard to the introduction of exogenous nucleic acids into animal cells, and the term “transfection” will be used herein to generally encompass both transfection of animal cells and transformation of microbial cells, to the extent that the terms pertain to the introduction of exogenous nucleic acids into a cell. Therefore, transfection techniques include, but are not limited to, transformation, electroporation, microinjection, lipofection, adsorption, infection and protoplast fusion.

### Compositions

Some embodiments of the present invention include a composition or formulation for diagnostic, screening or therapeutic purposes. Such compositions or formulations can include any antibodies against Mer glycoforms as described herein, any Mer polypeptides (e.g., soluble forms of Mer and/or any Mer glycoforms described herein), or any nucleic acid molecules encoding Mer and particularly, any of the soluble Mer proteins or Mer glycoforms of the invention). In one aspect, the agents described above can be formulated with a pharmaceutically acceptable carrier. The phrase “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Common suitable pharmaceutical carriers are described in “Remington's Pharmaceutical Sciences” by E. W. Martin.

According to the invention, the pharmaceutical composition of the invention can be introduced parenterally, transmucosally, e.g., orally (per os), nasally or transdermally. Parental routes include intravenous, intra-arteriole, intramuscular, intradermal, subcutaneous, intraperitoneal, intraventricular and intracranial administration. Preferably, administration is directly into the cerebrospinal fluid, e.g., by a spinal tap.

In another embodiment, the therapeutic compound can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss: New York, pp. 353-365 (1989). To reduce its systemic side effects, this may be a preferred method for introducing the compound.

In yet another embodiment, the therapeutic compound can be delivered in a controlled release system. For example, a polypeptide may be administered using intravenous infusion with a continuous pump, in a polymer matrix such as poly-lactic/glutamic acid (PLGA), a pellet containing a mixture of cholesterol and the anti-amyloid peptide antibody compound (U.S. Pat. No. 5,554,601) implanted subcutaneously, an implantable osmotic pump, a transdermal patch, liposomes, or other modes of administration.

The pharmaceutical compositions of the invention may further comprise a therapeutically effective amount of the monoclonal antibodies of the invention, or the soluble extracellular form of Mer and/or Mer glycoform, preferably in respective proportions such as to provide a synergistic effect in the said prevention or treatment. A therapeutically effective amount of an pharmaceutical composition of the invention relates generally to the amount needed to achieve a therapeutic objective.

### Methods of the Invention

The present invention also includes a variety of diagnostic, prognostic and therapeutic methods that particularly make use of the Mer glycoforms discovered by the present inventors, as well as the agents disclosed herein, including antibodies of the present invention, Mer glycoforms, soluble Mer proteins (particularly soluble Mer glycoforms), and nucleic acid molecules encoding soluble Mer proteins and Mer proteins having modified glycosylation sites.

Diagnostic Methods of the Invention

Accordingly, one embodiment of the present invention provides a method of diagnosing cancers, such as leukemias or lymphomas in an individual, comprising detecting specific glycoforms of the Mer transmembrane receptor tyrosine kinase in a patient sample, wherein the presence of a specific glycoforms of the Mer transmembrane receptor tyrosine kinase that is associated with a particular cancer is indicative of the cancer, or wherein the presence of certain specific glycoforms are prognostic markers for the tractability of various therapeutic methods. Diagnostic assays of the present invention are designed to assess aberrant Mer glycoforms. In one embodiment, the assays are used in the context of a biological sample (e.g., blood, serum, cells, tissue) to thereby determine whether an individual is afflicted with a cancer, and in a preferred embodiment, leukemia or lymphoma, and the severity of the disease.

The method of the invention includes the step of detecting an aberrant glycoform(s) of Mer transmembrane receptor tyrosine kinase in an individual, wherein the presence of the aberrant glycoform(s) of the Mer transmembrane receptor tyrosine kinase is indicative of the presence of a cancer cell that expresses the aberrant glycoform of Mer in the individual.

According to the present invention, the phrase “tumorigenicity” refers primarily to the tumor status of a cell or cells (i.e., the extent of neoplastic transformation of a cell, the malignancy of a cell, or the propensity for a cell to form a tumor and/or have characteristics of a tumor), which is a change of a cell or population of cells from a normal to malignant state. Tumorigenicity indicates that tumor cells are present in a sample, and/or that the transformation of cells from normal to tumor cells is in progress, as may be confirmed by any standard of measurement of tumor development. The change typically involves cellular proliferation at a rate which is more rapid than the growth observed for normal cells under the same conditions, and which is typically characterized by one or more of the following traits: continued growth even after the instigating factor (e.g., carcinogen, virus) is no longer present; a lack of structural organization and/or coordination with normal tissue, and typically, a formation of a mass of tissue, or tumor. A tumor, therefore, is most generally described as a proliferation of cells (e.g., a neoplasia, a growth, a polyp) resulting from neoplastic growth and is most typically a malignant tumor. In the case of a neoplastic transformation, a neoplasia is malignant or is predisposed to become malignant. Malignant tumors are typically characterized as being anaplastic (primitive cellular growth characterized by a lack of differentiation), invasive (moves into and destroys surrounding tissues) and/or metastatic (spreads to other parts of the body). As used herein, reference to a “potential for neoplastic transformation”, “potential for tumorigenicity” or a “potential for tumor cell growth” refers to an expectation or likelihood that, at some point in the future, a cell or population of cells will display characteristics of neoplastic transformation, including rapid cellular proliferation characterized by anaplastic, invasive and/or metastatic growth. In the present invention, the expectation or likelihood of tumorigenicity or neoplastic transformation and particularly malignant tumor cell growth (i.e., a positive diagnosis of tumorigenicity) is determined based on a detection of aberrant expression of a specific Mer glycoform(s) in a cell.

This method of the present invention has several different uses. First, the method can be used to diagnose the presence or absence of tumor cells of a particular type, in a subject. The subject can be an individual who is suspected of having a tumor, or an individual who is presumed to be healthy, but who is undergoing a routine or diagnostic screening for the presence of a tumor (cancer). The subject can also be an individual who has previously been diagnosed with cancer and treated, and who is now under surveillance for recurring tumor growth. The terms “diagnose”, “diagnosis”, “diagnosing” and variants thereof refer to the identification of a disease or condition on the basis of its signs and symptoms. As used herein, a “positive diagnosis” indicates that the disease or condition, or a potential for developing the disease or condition, has been identified. In contrast, a “negative diagnosis” indicates that the disease or condition, or a potential for developing the disease or condition, has not been identified. Therefore, in the present invention, a positive diagnosis (i.e., a positive assessment) of tumor growth or tumorigenicity (i.e., malignant or inappropriate cell growth or neoplastic transformation), or the potential therefore, means that the indicators (e.g., signs, symptoms) of tumor presence and/or growth according to the present invention (i.e., expression of a particular Mer glycoform) has been identified in the sample obtained from the subject. Such a subject can then be prescribed treatment to reduce or eliminate the tumor growth. Similarly, a negative diagnosis (i.e., a negative assessment) for tumor growth or a potential therefore or the absence of tumor cells means that the indicators of tumor growth or tumor presence or a likelihood of developing tumors as described herein (i.e., no detection of Mer or a particular Mer glycoform) have not been identified in the sample obtained from the subject. In this instance, the subject is typically not prescribed any treatment, but may be reevaluated at one or more timepoints in the future to again assess tumor growth.

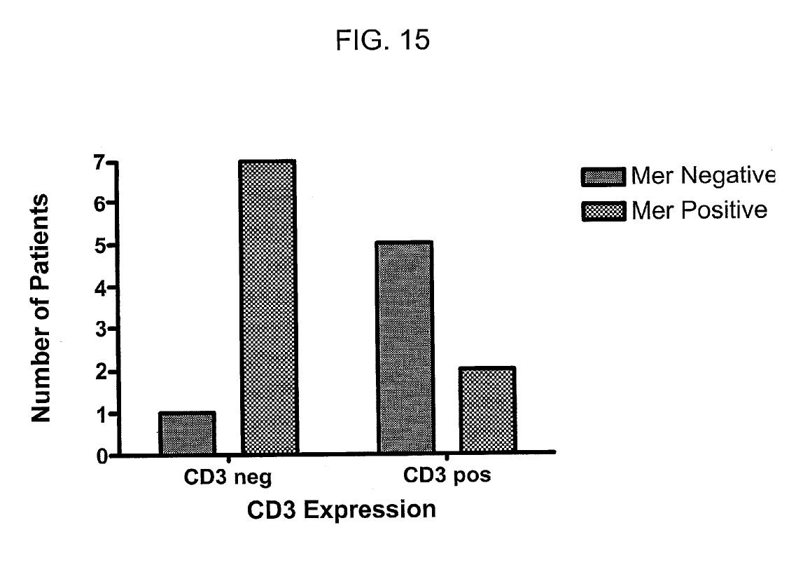

In another embodiment of the invention, Mer glycoform expression has prognostic significance for cancer patients, and particularly, for leukemia patients. For example, the present inventors have discovered that patients diagnosed with T cell acute lymphoblastic leukemia (ALL) having high levels of Mer expression also had lymphoblasts which were negative for CD3 expression, suggesting that the leukemia arose from an immature stage of thymocyte differentiation (FIG. 15). CD3 negative (i.e., immature stage) T cell leukemias have a decreased event free survival, unless chemotherapy is intensified. The association of Mer with the CD3 negative T cell ALL subset suggests a prognostic significance for Mer expression in T cell ALL. Therefore, in addition to detecting Mer glycoforms, the type and level of Mer glycoform expression can be used to determine a prognosis for certain cancer patients.

Preferred cancers to diagnose using the methods of the present invention include any cancers wherein tumor cells have been correlated with expression of Mer and specifically, an aberrant glycoform of Mer. Preferred cancers to diagnose using the method of the invention include, but are not limited to, leukemia and lymphoma, and more particularly, lymphoblastic leukemias, including acute lymphoblastic leukemia (ALL), and myelogenous leukemia. In particular, detection of a Mer glycoform having a molecular weight of between about 170 kD and about 190 kD and/or from about 135 kD to about 140 kD indicates a positive diagnosis of lymphoblastic leukemia. Detection of a Mer glycoform having a molecular weight of between about 190 kD to about 195 kD indicates a positive diagnosis of myelogenous leukemia. Furthermore, detection of high levels of ectopic Mer RNA transcript in lymphoblasts by PCR or quantitative PCR or detection of a Mer glycoform in lymphoblasts having a molecular weight of between about 170 kD and about 190 kD by Western blot or flow cytometry in combination with lack of surface CD3, indicates a positive diagnosis for lymphoblastic leukemia that has a decreased event free survival, unless chemotherapy is intensified.

The method of the present invention includes detecting Mer glycoform expression in a test sample from a subject. According to the present invention, the term “test sample” can be used generally to refer to a sample of any type which contains cells or products that have been secreted from cells to be evaluated by the present method, including but not limited to, a sample of isolated cells, a tissue sample and/or a bodily fluid sample. According to the present invention, a sample of isolated cells is a specimen of cells, typically in suspension or separated from connective tissue which may have connected the cells within a tissue in vivo, which have been collected from an organ, tissue or fluid by any suitable method which results in the collection of a suitable number of cells for evaluation by the method of the present invention. A cell sample can also be processed to obtain a soluble product therefrom, such as a supernatant or lysate from the cell that might contain a soluble Mer protein. The cells in the cell sample are not necessarily of the same type, although purification methods can be used to enrich for the type of cells that are preferably evaluated. Cells can be obtained, for example, by scraping of a tissue, processing of a tissue sample to release individual cells, or isolation from a bodily fluid. A tissue sample, although similar to a sample of isolated cells, is defined herein as a section of an organ or tissue of the body which typically includes several cell types and/or cytoskeletal structure which holds the cells together. One of skill in the art will appreciate that the term “tissue sample” may be used, in some instances, interchangeably with a “cell sample”, although it is preferably used to designate a more complex structure than a cell sample. A tissue sample can be obtained by a biopsy, for example, including by cutting, slicing, or a punch. A bodily fluid sample, like the tissue sample, contains the cells to be evaluated for Mer glycoform expression, and is a fluid obtained by any method suitable for the particular bodily fluid to be sampled. Bodily fluids suitable for sampling include, but are not limited to, blood, mucous, seminal fluid, saliva, breast milk, bile and urine. In general, the sample type (i.e., cell, tissue or bodily fluid) is selected based on the accessibility and structure of the organ or tissue to be evaluated for tumor cell growth and/or on what type of cancer is to be evaluated.

Once a sample is obtained from the subject, the sample is evaluated for detection of Mer glycoform expression in the cells of the sample. The phrase “Mer expression” (including as it applies to either form of Mer) can generally refer to Mer mRNA transcription or Mer protein translation. Detection of Mer transcription is useful to detect whether or not a given cell expresses Mer, but is not useful for detecting Mer glycoforms unless the Mer glycoforms are due to a mutation in the nucleic acid sequence encoding Mer that results in a modification of glycosylation sites in the expressed protein. However, detection of other variations in Mer may be combined with the detection of Mer glycoforms to enhance the diagnostic potential of the method of the present invention.

Accordingly, methods suitable for detecting Mer transcription include any suitable method for detecting and/or measuring mRNA levels from a cell or cell extract. Such methods include, but are not limited to: polymerase chain reaction (PCR), reverse transcriptase PCR(RT-PCR), in situ hybridization, Northern blot, sequence analysis, gene microarray analysis (gene chip analysis) and detection of a reporter gene. Such methods for detection of transcription levels are well known in the art, and many of such methods are described in detail in the attached examples, in Sambrook et al., Molecular Cloning: A Laboratory Manual, Cold Spring Harbor Labs Press, 1989 and/or in Glick et al., Molecular Biotechnology: Principles and Applications of Recombinant DNA, ASM Press, 1998; Sambrook et al., ibid., and Glick et al., ibid. are incorporated by reference herein in their entireties.

Mer glycoform expression is more typically identified by detection of Mer translation (i.e., detection of Mer protein in a sample). Methods suitable for the detection of Mer protein include any suitable method for detecting and/or measuring proteins from a cell or cell extract. Such methods include, but are not limited to, immunoblot (e.g., Western blot), enzyme-linked immunosorbant assay (ELISA), radioimmunoassay (RIA), immunoprecipitation, immunohistochemistry and immunofluorescence. Particularly preferred methods for detection of proteins include any single-cell assay, including immunohistochemistry and immunofluorescence assays. Such methods are well known in the art. Furthermore, antibodies against Mer that are particularly useful for the detection of Mer glycoforms are provided by the present invention and can be used in these methods.

A positive diagnosis of the target Mer glycoform in the individual sample indicates that tumor cell growth (neoplastic transformation), has occurred, is occurring, or is statistically likely to occur in the cells or tissue from which the sample was obtained. A negative diagnosis of the target Mer glycoform in the individual sample (i.e., the Mer glycoform was not detected) means that the indicators of tumor presence or a likelihood of developing tumors as described herein have not been identified in the sample obtained from the subject. In this instance, the subject is typically not prescribed any treatment, but may be reevaluated at one or more timepoints in the future to again assess tumor growth.

The nucleic acids, probes, primers, polypeptides (including soluble Mer and Mer glycoforms) and antibodies described herein can be used in methods of diagnosis of cancers, and particularly, leukemias and lymphomas, as well as in kits useful for diagnosis of such cancers, particularly leukemias and lymphomas. For example, the invention provides methods for identifying the presence of a polynucleotide that hybridizes to a nucleic acid of the invention (i.e., nucleic acids encoding aberrant Mer glycoforms or the soluble extracellular Mer), as well as for identifying the presence of a polypeptide of the invention (e.g., aberrant Mer glycoforms or the soluble extracellular Mer), using agents that detect such Mer glycoforms or soluble Mer (e.g., antibodies or antigen binding fragments thereof).

In one embodiment, the presence (or absence) of a nucleic acid molecule of interest (e.g., a nucleic acid that has significant homology with a nucleic acid of the invention) in a sample can be assessed by contacting the sample with a nucleic acid comprising a nucleic acid of the invention (e.g., a nucleic acid encoding an aberrant Mer glycoform or the soluble extracellular Mer) under stringent conditions as described above, and then assessing the sample for the presence (or absence) of hybridization. In a preferred embodiment, high stringency conditions are conditions appropriate for selective hybridization. In another embodiment, a sample containing the nucleic acid molecule of interest is contacted with a nucleic acid containing a contiguous nucleotide sequence (e.g., a primer or a probe as described above) that is at least partially complementary to a part of the nucleic acid molecule of interest), and the contacted sample is assessed for the presence or absence of hybridization. In a preferred embodiment, the nucleic acid containing a contiguous nucleotide sequence is completely complementary to a part of the nucleic acid molecule of interest. In any of these embodiments, all or a portion of the nucleic acid of interest can be subjected to amplification prior to performing the hybridization.

More particularly, in one method of diagnosing leukemia or lymphoma, hybridization methods, such as Southern analysis, Northern analysis, or in situ hybridizations, can be used. Such techniques of detection are well known in the art, see, e.g., Current Protocols in Molecular Biology, Ausubel, F. et al., eds., John Wiley & Sons, including all current supplements. For example, a biological sample from a test subject (a “test sample”) of genomic DNA, RNA, or cDNA, is obtained from an individual suspected of having leukemia (the “test individual”). The test sample can be from any source which contains genomic DNA, such as a blood sample, sample of amniotic fluid, sample of cerebrospinal fluid, or tissue sample from skin, muscle, buccal or conjunctival mucosa, placenta, gastrointestinal tract or other organs. The DNA, RNA, or cDNA sample is then examined to determine whether nucleic acid encoding variant isoforms, including but not limited to Mer isoforms in the 170 kD to 195 kD range, is present. Such a variant will be detected by this method when the glycosylation of the Mer protein results from a modification in the glycosylation sites of the protein expressed by the nucleic acid sequence from the patient sample (e.g., the nucleic acid encoding Mer is a variant). The presence of the isoform or splicing variant(s) can be indicated by hybridization of the gene in the genomic DNA, RNA, or cDNA to a nucleic acid probe. A “nucleic acid probe”, as used herein, can be a DNA probe or an RNA probe. The probe can be any of the nucleic acid molecules described above (e.g., the gene, a fragment, a vector comprising the gene, a probe or primer, etc.).

The sample is maintained under conditions that are sufficient to allow specific hybridization. “Specific hybridization”, as used herein, indicates exact hybridization (e.g., with no mismatches). Specific hybridization can be performed under high stringency conditions or moderate stringency conditions, for example, as described above. In a particularly preferred embodiment, the hybridization conditions for specific hybridization are high stringency. Hybridization is then detected by standard methods well known in the art.

Sequence analysis can also be used to detect mutations in Mer causing size differences. A test sample of DNA or RNA is obtained from the test individual. PCR or other appropriate methods can be used to amplify the gene, and/or its flanking sequences, if desired. The sequence of Mer, or a fragment of the gene, or cDNA, or fragment of the cDNA, or mRNA, or fragment of the mRNA, is determined, using standard methods. The sequence of the gene, gene fragment, cDNA, cDNA fragment, mRNA, or mRNA fragment is compared with the known nucleic acid sequence of the gene.

When aberrant glycoforms are due to mutations in the Mer gene, oligonucleotides produced from the nucleic acids of the invention that detect such differences can also be used to detect the presence of aberrant Mer glycoforms, through the use of dot-blot hybridization. An “oligonucleotide” (also referred to herein as a “probe”) is a nucleic acid of approximately 10-50 base pairs, preferably approximately 15-30 base pairs, that specifically hybridizes to the sequence coding for the aberrant Mer glycoform. An oligonucleotide probe that is specific for the aberrant Mer glycoform can be prepared, using standard methods (see Current Protocols in Molecular Biology, supra). To do this, a test sample of DNA is obtained from the individual. PCR can be used to amplify all or a fragment of Mer, and its flanking sequences. The DNA containing the amplified Mer (or fragment of the gene) is dot-blotted, using standard methods (see Current Protocols in Molecular Biology, supra), and the blot is contacted with the oligonucleotide probe. The presence of specific hybridization of the probe to the aberrant Mer glycoform is then detected. Specific hybridization of an oligonucleotide probe to DNA from the individual is suggestive of leukemia or lymphoma.

Other methods of nucleic acid analysis can be directed to the sequence coding for the aberrant Mer glycoform. Representative methods include direct manual sequencing (Church and Gilbert, Proc. Natl. Acad. Sci. USA 81:1991-1995 (1988); Sanger, F. et al., Proc. Natl. Acad. Sci. 74:5463-5467 (1977); Beavis et al. U.S. Pat. No. 5,288,644); automated fluorescent sequencing; single-stranded conformation polymorphism assays (SSCP); clamped denaturing gel electrophoresis (CDGE); denaturing gradient gel electrophoresis (DGGE) (Sheffield, V. C. et al., Proc. Natl. Acad. Sci. USA 86:232-236 (19891)), mobility shift analysis (Orita, M. et al., Proc. Natl. Acad. Sci. USA 86:2766-2770 (1989)), restriction enzyme analysis (Flavell et al., Cell 15:25 (1978); Geever, et al., Proc. Natl. Acad. Sci. USA 78:5081 (1981)); heteroduplex analysis; chemical mismatch cleavage (CMC) (Cotton et al., Proc. Natl. Acad. Sci. USA 85:4397-4401 (1985)); RNase protection assays (Myers, R. M. et al., Science 230:1242 (1985)).

In another embodiment, the presence (or absence) of a polypeptide of interest, such as a polypeptide of the invention or a fragment or variant thereof, in a sample can be assessed by contacting the sample with an antibody that specifically hybridizes to the polypeptide of interest (e.g., an antibody such as those described above), and then assessing the sample for the presence (or absence) of binding of the antibody to the polypeptide of interest For example, diagnosis of cancer by diagnosing an aberrant Mer glycoform, soluble Mer, or a Mer variant, as in leukemia or lymphoma, can be made by examining expression and/or composition of the Mer polypeptide, by a variety of methods, including enzyme linked immunosorbent assays (ELISAs), Western blots, immunoprecipitations and immunofluorescence. A test sample from an individual is assessed for the presence aberrant Mer glycoforms, or for the presence of a particular variant. In a preferred embodiment, any of the antibodies or antigen binding fragments thereof described herein can be used such methods of the invention.

In one embodiment, Western blotting analysis, e.g., using the 311 monoclonal antibody as described herein or other antibodies that identify all glycoforms of Mer or specifically identify the aberrant glycoform of Mer as described herein can be used to identify the presence in a test sample of any in the spectrum of Mer glycoforms or of soluble Mer in a sample. Other techniques to identify the Mer glycoforms will be known in the art and some are described elsewhere herein. The presence of a Mer glycoform from about 170 kD to about 190 kD (and particularly, 185 kD) or a Mer glycoform from about 135 kD to about 140 kD (and particularly 140 kD) is diagnostic of leukemia (acute lymphoblastic leukemia). The presence of the 190-195 kD Mer glycoform is diagnostic of myelogenous leukemia, or lymphoma.

Kits (e.g., reagent kits) useful in the methods of diagnosis comprise components useful in any of the methods described herein, including for example, soluble Mer proteins, including soluble Mer glycoforms described herein, cells expressing such Mer proteins and glycoforms, hybridization probes or primers as described herein (e.g., labeled probes or primers), reagents for detection of labeled molecules, restriction enzymes (e.g., for RFLP analysis), allele-specific oligonucleotides, antibodies and antigen-binding fragments thereof, means for amplification of nucleic acids, or means for analyzing nucleic acid or amino acid sequences, positive controls etc.

Screening Assays of the Invention

In another embodiment, the invention provides methods and screening assays for identifying agents (e.g., fusion proteins, polypeptides, peptidomimetics, prodrugs, receptors, binding agents, antibodies, small molecules or other drugs, ribozymes, or nucleic acids) that alter (e.g., increase or decrease), mimic or regulate the activity of the sMer, or aberrant Mer glycoforms or which otherwise interact with these polypeptides described herein. For example, such agents can be agents which bind to polypeptides described herein; which have a stimulatory or inhibitory effect on, for example, activity of polypeptides of the invention; or which change (e.g., enhance or inhibit) the ability of the polypeptides of the invention to interact with binding agents (e.g., receptors or other binding agents). For example, the ability of a molecule to affect the binding of the extracellular domain of Mer to its ligands presents a screening assay for molecules that affect blood clotting or for molecules that can be used to treat a particular cancer. Similarly, the ability of a molecule to inhibit the proteolysis that releases Mer from its cellular anchor, as exemplified by TAPI (see FIG. 11), presents a screening assay for molecules that affect blood clotting from an alternative direction. In another example, a screening assay using the sMer can identify molecules which bind the sMer and therefore subsequently modulate (inhibit, interfere with, enhance, or otherwise affect) the Mer tyrosine kinase and inhibit the activity of Mer in cancer. For example, such assays can be in the form of examining Mer phosphorylation or glycosylation (or lack thereof) (FIG. 13), and the effect on this activation on cell survival and cell proliferation. This invention also contemplates an assay for identifying protease inhibitors, more preferably a metalloprotease inhibitor and most preferably TACE inhibitors.

In a preferred embodiment, such assays take advantage of the discovery by the present inventors of the differential expression of various glycoforms of Mer. In particular, methods of the present invention can make use of particular glycoforms of Mer, including any specific glycoforms described herein, to screen for compounds (proteins, peptides, antibodies, small molecules, etc.) that selectively bind to a particular Mer glycoform (e.g., to one glycoform preferentially over another). In addition, methods of the present invention can be used to screen for Mer glycoforms that competitively inhibit the binding of a Mer ligand to its endogenous Mer receptor and that can be used as therapeutic agent. The methods of the present invention can also be used to screen for Mer proteins (including specific Mer glycoforms) that selectively (preferentially) bind to one Mer ligand (e.g., Gas6 or Protein S) over another Mer ligand.

In one embodiment, several different Mer glycoforms (e.g., any combination or all of the glycoforms described herein) are contacted with Mer ligands or other putative regulatory compounds to identify differences in binding affinity or Mer activity (in the case of a cell-based assay) in response to the contact with the ligand or compound. In another embodiment, additional glycoforms of Mer other than those described herein (e.g., having any molecular weight, as a result of glycosylation, from between about 109 kD and 210 kD or larger (in the case of Mer variants where additional glycosylation sites are added to the protein as compared to the wild-type protein), are tested for binding to Mer ligands or putative regulatory compounds. Specifically, any Mer glycoform from 100 kd to 210 kD or larger, including any size between 100 kD and 210 kD or larger, in increments of 1 kD (i.e., 100 kD, 101 kD, 102 kD, . . . 145 kD, 146 kD, . . . 201 kD, 202 kD, . . . 210 kD, 211 kD, . . . etc.) can be tested to identify Mer glycoforms that have preferred affinity for Mer ligands or putative regulatory compounds. Mer glycoforms with differential affinity for Mer ligands, or that bind to Mer ligands with a greater affinity than a natural Mer glycoform expressed by a target cell type (e.g., a platelet or a tumor cell) are particularly desirable, as these glycoforms can be produced as soluble Mer glycoforms for use in various therapeutic assays (e.g., as competitive inhibitors of endogenous Mer to treat a condition such as a cancer or a clotting disorder).

This invention also contemplates an assay for identifying portions of extracellular domain of Mer functions in modulating Gas6 functionalities and where the Mer domain interacts with Gas6, as well as identifying small molecule mimetics that would also interact at the same Gas6 domains.

Another aspect of the invention pertains to assays for monitoring the influence of agents (e.g., drugs, compounds or other agents) on the gene expression or activity of polypeptides of the invention, as well as to assays for identifying agents that bind to or inhibit aberrant Mer glycoforms and the soluble Mer polypeptides of the invention.

Such a method includes the steps of: (a) contacting a Mer protein with a test compound; and (b) determining whether the test compound binds to the Mer protein. The method can optionally include an additional step of determining whether the test compound activates or inhibits the biological activity of a Mer receptor, by detecting, for example, signal transduction events that are associated with Mer tyrosine kinase activity. The assay can be cell-based, in the event where Mer activity is evaluated, or non-cell based, in the event where only binding is evaluated.

A cell suitable for use in the present method is any cell which expresses or can be induced to express, a detectable level of Mer, and particularly, a specific glycoform of Mer. A detectable level of Mer is a level which can be detected using any of the methods for Mer detection described herein. Since Mer is expressed by many mammalian cell types, a variety of cell types could be selected. Preferred cell types that endogenously express particular glycoforms of Mer are described herein. Alternatively, a cell suitable for use in the method is a cell which has been transfected with a recombinant nucleic acid molecule encoding Mer and operatively linked to a transcription control sequence so that Mer is expressed by the cell. The cell can be selected to post-translationally modify the Mer protein to produce a particular glycoform of Mer, or the nucleic acid molecule encoding the Mer protein can be modified so that glycosylation sites on the Mer protein are designed to produce a particular Mer glycoform. Methods and reagents for preparing recombinant cells are known in the art.

For non-cell-based assays, soluble Mer glycoforms can be produced using any method described herein and immobilized on a suitable substrate or mixed with a test compound in an assay container.

As used herein, the term “putative regulatory compound” refers to compounds having an unknown or previously unappreciated regulatory activity in a particular process. The above-described method for identifying a compound of the present invention includes a step of contacting a test compound with a Mer receptor and particularly, a specified Mer glycoform. When cells expressing Mer are used, test cells can be grown in liquid culture medium or grown on solid medium in which the liquid medium or the solid medium contains the compound to be tested. In addition, the liquid or solid medium contains components necessary for cell growth, such as assimilable carbon, nitrogen and micronutrients.

The above-described methods, in one aspect, involve contacting cells with the compound being tested for a sufficient time to allow for interaction of the putative regulatory compound with the Mer glycoform. The period of contact with the compound being tested can be varied depending on the result being measured, and can be determined by one of skill in the art. For example, for binding assays, a shorter time of contact with the compound being tested is typically suitable, than when activity is assessed. As used herein, the term “contact period” refers to the time period during which cells are in contact with the compound being tested. The term “incubation period” refers to the entire time during which cells are allowed to grow prior to evaluation, and can be inclusive of the contact period. Thus, the incubation period includes all of the contact period and may include a further time period during which the compound being tested is not present but during which growth is continuing (in the case of a cell based assay) prior to scoring. The incubation time for growth of cells can vary but is sufficient to allow for the upregulation or downregulation of Mer biological activity in a cell. It will be recognized that shorter incubation times are preferable because compounds can be more rapidly screened. A preferred incubation time is between about 1 hour to about 48 hours.

The conditions under which the cell or cell lysate of the present invention is contacted with a putative regulatory compound, such as by mixing, are any suitable culture or assay conditions and includes an effective medium in which the cell can be cultured or in which a soluble Mer or immobilized Mer can be evaluated in the presence and absence of a putative regulatory compound. Cells of the present invention can be cultured in a variety of containers including, but not limited to, tissue culture flasks, test tubes, microtiter dishes, and petri plates. Culturing is carried out at a temperature, pH and carbon dioxide content appropriate for the cell. Such culturing conditions are also within the skill in the art. Cells are contacted with a putative regulatory compound under conditions which take into account the number of cells per container contacted, the concentration of putative regulatory compound(s) administered to a cell, the incubation time of the putative regulatory compound with the cell, and the concentration of compound administered to a cell. Determination of effective protocols can be accomplished by those skilled in the art based on variables such as the size of the container, the volume of liquid in the container, conditions known to be suitable for the culture of the particular cell type used in the assay, and the chemical composition of the putative regulatory compound (i.e., size, charge etc.) being tested. A preferred amount of putative regulatory compound(s) comprises between about 1 nM to about 10 mM of putative regulatory compound(s) per well of a 96-well plate.

Mer proteins and nucleic acid molecules encoding Mer may be recombinantly expressed and utilized in non-cell based assays to identify compounds that bind to the protein or nucleic acid molecule, respectively. In non-cell based assays the recombinantly expressed Mer or nucleic acid encoding Mer is attached to a solid substrate such as a test tube, microtiter well or a column, by means well known to those in the art.

Compounds suitable for testing and use in the methods of the present invention include any known or available proteins, nucleic acid molecules, as well as products of drug design, including peptides, oligonucleotides, carbohydrates and/or synthetic organic molecules. Such an agent can be obtained, for example, from molecular diversity strategies (a combination of related strategies allowing the rapid construction of large, chemically diverse molecule libraries), libraries of natural or synthetic compounds, in particular from chemical or combinatorial libraries (i.e., libraries of compounds that differ in sequence or size but that have the same building blocks) or by rational drug design. See for example, Maulik et al., 1997, Molecular Biotechnology: Therapeutic Applications and Strategies, Wiley-Liss, Inc., which is incorporated herein by reference in its entirety. Candidate compounds initially identified by drug design methods can be screened for the ability to modulate the expression and/or biological activity of Mer using the methods described herein.

Compounds identified by the method described above can be used in a method to regulate cell growth or thrombosis, as described below and any such compounds are encompassed for use in the method described below.

Therapeutic Methods of the Invention

The present invention also relates to methods of treatment (prophylactic and/or therapeutic) for Mer-positive cancers and clotting disorders, using the polypeptides (including sMer and particularly, specific glycoforms of Mer, and more particularly, specific glycoforms of sMer), nucleic acid molecules, and/or antibodies of the invention.

As used herein, “treatment” refers to clinical intervention in an attempt to alter the natural course of the individual or cell being treated, and may be performed either for prophylaxis or during the course of clinical pathology. Desirable effects include preventing occurrence or recurrence of disease, alleviation of symptoms, diminishment of any direct or indirect pathological consequences of the disease, preventing metastasis, lowering the rate of disease progression, amelioration or palliation of the disease state, and remission or improved prognosis. Accordingly, a therapeutic benefit is not necessarily a cure for a particular disease or condition, but rather, preferably encompasses a result which most typically includes alleviation of the disease or condition, elimination of the disease or condition, reduction of a symptom associated with the disease or condition, prevention or alleviation of a secondary disease or condition resulting from the occurrence of a primary disease or condition (e.g., metastatic tumor growth resulting from a primary cancer), and/or prevention of the disease or condition. A beneficial effect can easily be assessed by one of ordinary skill in the art and/or by a trained clinician who is treating the patient. The term, “disease” refers to any deviation from the normal health of a mammal and includes a state when disease symptoms are present, as well as conditions in which a deviation (e.g., infection, gene mutation, genetic defect, etc.) has occurred, but symptoms are not yet manifested.

According to the present invention, the methods and assays disclosed herein are suitable for use in or with regard to an individual that is a member of the Vertebrate class, Mammalia, including, without limitation, primates, livestock and domestic pets (e.g., a companion animal). Most typically, a patient will be a human patient. According to the present invention, the terms “patient”, “individual” and “subject” can be used interchangeably, and do not necessarily refer to an animal or person who is ill or sick (i.e., the terms can reference a healthy individual or an individual who is not experiencing any symptoms of a disease or condition).

Diseases and disorders that are characterized by altered (relative to a subject not suffering from the disease or disorder) Mer receptor tyrosine kinases, levels of this protein, or biological activity may be treated with therapeutics that antagonize (e.g., reduce or inhibit) the altered Mer receptor tyrosine kinase or its ligands. For example, the soluble extracellular form of Mer may block the activation of the full length native Mer or aberrant glycoforms of Mer by binding to Mer ligands including Protein S and Gas6. Therefore, in another embodiment of the invention, an effective amount of an inhibitor of a Protein S or Gas6 function or of a Gas6 receptor which is provided in the form of the soluble extracellular form of Mer herein described, and particularly, in the form of a particular soluble glycoform of Mer, may be used as a treatment for diseases and conditions associated with Mer expression, including aberrant Mer expression.

Accordingly, an additional aspect of the invention pertains to methods of modulating expression or activity of Mer glycoforms. In one aspect, this is achieved by modulating the ability of ligands of Mer, including Protein S and Gas 6, to bind to endogenous Mer receptors, for therapeutic purposes. The method of the invention for example, involves contacting a cell with an agent that modulates one or more of the activities of Mer. One such agent includes an anti-Mer antibody or antigen-binding fragment thereof that binds to and inhibits Mer activity in a cell. Another agent that modulates Mer activity is preferably a soluble Mer protein described herein and particularly, a soluble Mer glycoform that binds to Mer ligands and serves as a competitive inhibitor of Mer expressed by cells. Additionally, nucleic acid molecules, peptides and small molecules that target specific Mer glycoforms can be used in therapeutic embodiments of the invention. These methods can be performed in vitro (e.g., by culturing the cell with the agent) or, alternatively, ex vivo or in vivo (e.g., by administering the agent to a subject). As such, the invention provides methods of treating an individual afflicted with a disease or disorder, specifically a clotting disorder or a cancer. In a preferred embodiment, the method involves administering a reagent (e.g., a specific glycoform of soluble extracellular Mer polypeptide described herein or an antibody that selectively binds to a glycoform of Mer), or combination of reagents that modulate Mer activity (e.g., binding and/or activation of Mer by a cognate ligand).

In one embodiment of the invention, modulation of specific Mer glycoforms is contemplated to prevent thrombosis or any clotting disorder without causing bleeding side effects. According to the present invention, “modulation” refers to any type of regulation, including upregulation, stimulation, or enhancement of expression or activity, or downregulation, inhibition, reduction or blocking of expression or activity. Preferably, the method of the present invention specifically inhibits the activity of the Mer glycoform expressed by platelets. Inhibition can be accomplished through various means, most preferably, but not limited to, by providing variants of the sMer, including differentially glycosylated Mer proteins and sMer proteins and peptides which bind directly to the extracellular domain of Mer or bind and competitively inhibit Mer ligands including Protein S and Gas6. Inhibition is also accomplished through antibodies or antigen-binding fragments thereof that are specific for the platelet glycoform of Mer, and most preferably the 165-170 kD glycoforms. Inhibition can also be achieved by providing to a patient small molecules, peptides, nucleic acids or other inhibitors which preferentially bind to and inhibit or block the glycosylated Mer extracellular domains that are expressed on platelets, or that bind to and inhibit or block the Mer ligands including protein S and Gas6. More preferably, all of these embodiments target the Mer glycoform that is specific to platelets as disclosed herein.

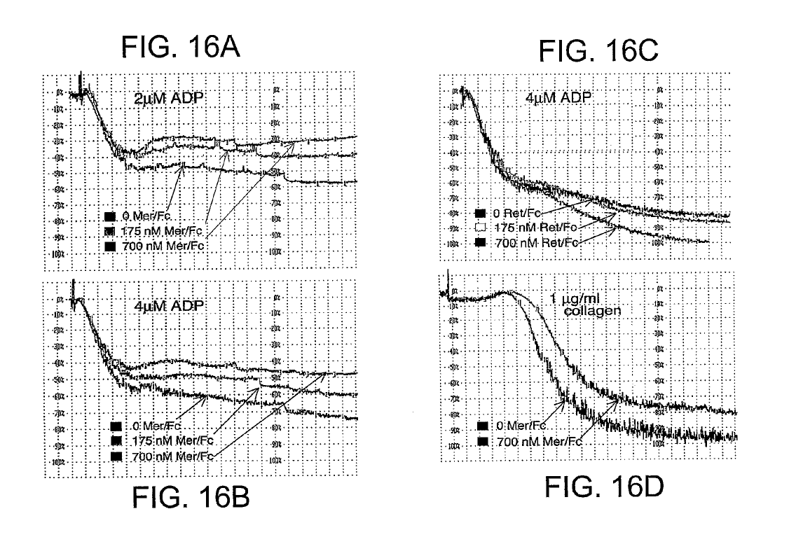

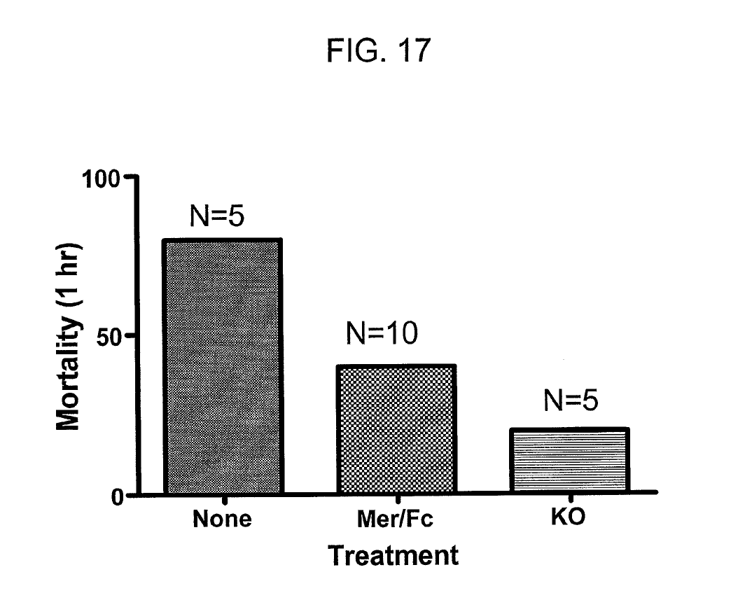

Modulation of Mer activity by administering antibodies to Mer ligand Gas6 has demonstrated protection of wild type mice against fatal thromboembolism to the same degree as genetic inactivation of this ligand, while not causing spontaneous bleeding. Further, the use of a version of sMer in the same mouse model as above inhibited platelet in vitro (FIG. 16) aggregation and protected against fatal pulmonary embolism in vivo (FIG. 17). Thus, the invention further contemplates modulating signaling through the Mer receptor as an anti-thrombotic approach, which is safer than currently available antiplatelet drugs. Thus, Mer, and in particular, the truncated or soluble form of Mer and its mimetics, and most preferably the soluble glycoform of Mer that is specific to platelets, are valuable prophylactic agents useful in the treatment and prevention of thrombotic events or disorders.

Clotting disorders that can be treated by the method of the invention include, but are not limited to, thrombophilia (including inherited traits predisposing an individual to have a higher risk of clotting), thrombosis or thrombo-embolic disorder. Specifically, this method of treatment could be applied to patients on medications (including, but not limited to, estrogens and chemotherapy) which increase the risk of clotting as well as diseases associated with thrombosis (including, but not limited to, cancer, myeloproliferative disorders, autoimmune disorders, cardiac disease, inflammatory disorders, atherosclerosis, hemolytic anemia, nephrosis, and hyperlipidemia). In addition, this method of treatment could be applied to predisposing factors to increased clotting including surgery, trauma, or pregnancy. Finally, this method of treatment may be appropriate for patients with adverse side effects from other anticoagulant or anti-platelet therapies, including heparin-induced thrombocytopenia (a severe immune-mediated drug reaction that occurs in 2-5% of patients exposed to heparin.)

Accordingly, the present invention provides for a method of treating an individual who has or is likely to develop a clotting disorder, comprising modulating the level of soluble extracellular Mer transmembrane receptor kinase in the blood. In one aspect, the invention includes a step of administering (e.g., by any suitable route, including infusion) an effective amount of a soluble extracellular Mer transmembrane receptor kinase into the individual, especially administration of the soluble extracellular domain of the Mer glycoform that is expressed by platelets (165-170 kD). An effective amount is any amount that achieves any detectable inhibition of the Mer receptor in the patient, or any detectable reduction in at least one symptom of the clotting disorder.

In another aspect, modulation occurs by administration of an agent that cleaves the extracellular domain of the Mer transmembrane receptor tyrosine kinase, preferably a metalloprotease and most preferably a TACE-like metalloprotease. Alternatively, modulation can be achieved by inhibiting cleavage of the Mer transmembrane receptor, preferably using a protease inhibitor, more preferably a metalloprotease inhibitor and most preferably using a TACE-like metalloprotease inhibitor.

In another aspect of the invention, clotting disorders are treated by administration of an agent that affects a post-translational proteolytic cleavage at the Mer extracellular domain. The cleaved extracellular domain becomes soluble after cleavage. This cleavage produces an inhibitory effect that is twofold: (1) competitive inhibition for Mer ligands (FIG. 6) and (2) eliminating functional activity of the Mer receptor tyrosine kinase (FIGS. 8A and 8B). In a preferred embodiment, the agent is specific for the glycoform of Mer that is expressed by platelets.

In one embodiment of the invention, the cleavage agent is a protease capable of cleaving Mer, including TACE or a TACE-like metalloprotease. In an additional aspect of the invention, an sMer produced by cleavage of the membrane bound Mer directly binds to Mer ligands, such as Protein S or Gas6, and prevents activation of the full length Mer receptor tyrosine kinase. Additionally, treatment of patients with a clotting disorder may occur by modulation of sMer levels in the blood. This may be accomplished by sMer infusion (particularly using sMer glycoforms of the invention) or targeting the metalloprotease activity affecting endogenous sMer levels.

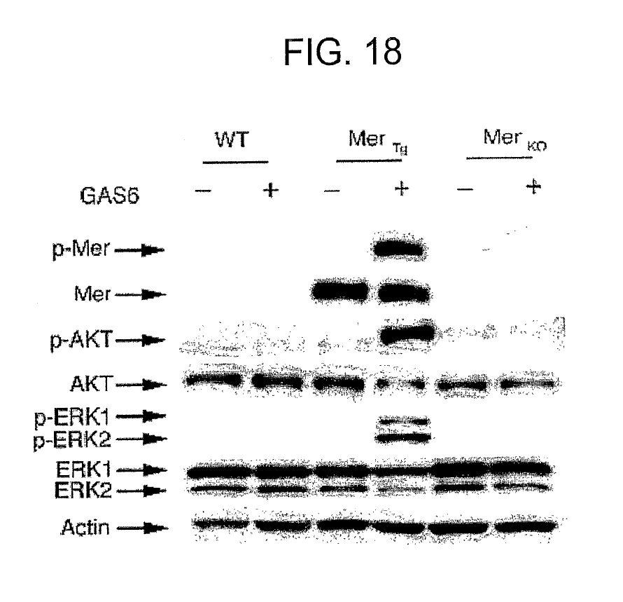

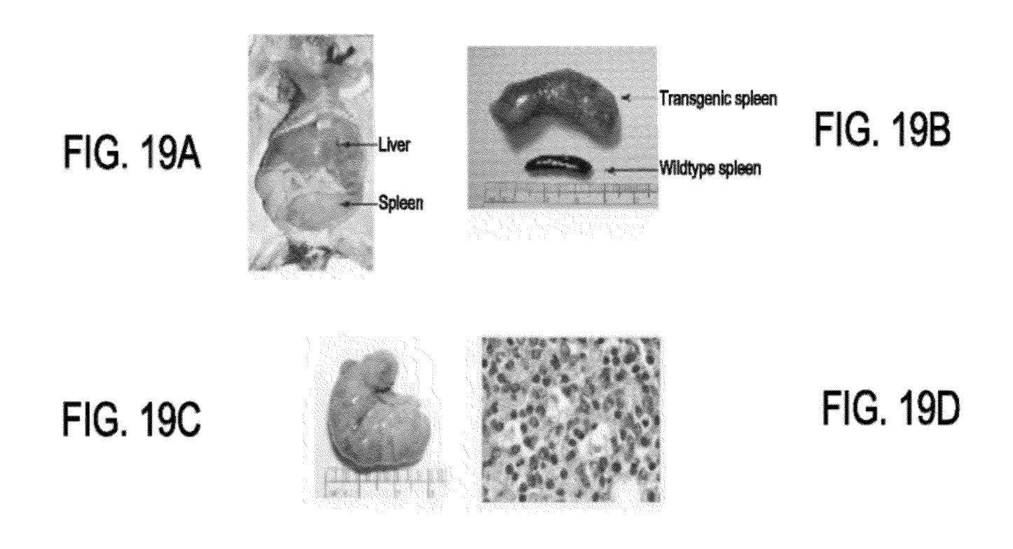

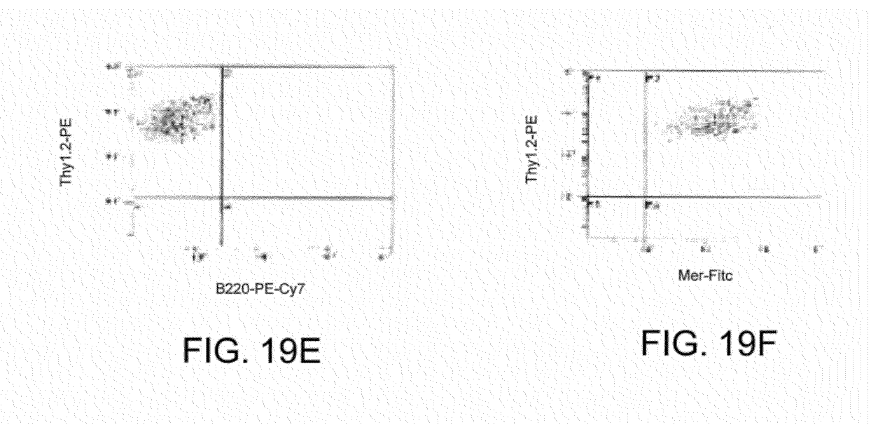

Mer activation leads to downstream activation of survival pathways (e.g., Akt and Erk ½) (FIG. 18), and in some instances proliferation pathways, that are known to contribute to the development of cancer. In some cancers, overexpression or ectopic expression of Mer glycoforms has been noted on the surface of cancer cells, as described herein. Furthermore, the overexpression of Mer and activation of downstream signaling pathways leads to lymphoblastic leukemia and lymphoma in a Mer transgenic mouse model (FIG. 19). The abnormal expression of Mer glycoforms makes this protein an attractive target for biologically targeted cancer therapy. Therefore, a further embodiment of the invention contemplates inhibition of the aberrantly glycosylated forms of the Mer receptor tyrosine kinase as part of a therapeutic strategy which selectively targets cancer cells. Any of the above-described methods and agents for treating a clotting disorder can be applied to the treatment of cancers. However, in these embodiments, the Mer that is targeted by the therapeutic method is preferably a Mer glycoform that is specifically expressed by a tumor cell (i.e., an aberrant Mer glycoform).

In one embodiment, the soluble extracellular portion of the Mer receptor tyrosine kinase, and particularly, a glycoform of sMer that selectively inhibits the Mer expressed by the target cancer cell, is used to block Mer activation and subsequent downstream signaling survival pathways, including AKT and Erk ½ as well as Mer specific proliferation pathways.

In another embodiment, small molecule inhibitors, nucleic acids, ribozymes, peptides or other drugs targeting aberrant Mer tyrosine kinase glycoforms or proteins directly downstream of Mer may be engineered and used in treatment regimens for Mer positive cancers. Antibodies (naked or coupled to toxins or radioactive materials) that bind to leukemia, lymphoma, or other cancer-specific forms of Mer protein can also be used therapeutically to treat Mer positive patients with cancer. For example, antibodies or antigen-binding fragments that are specific for (selectively bind to) the glycoforms of Mer that are expressed by the leukemia and lymphoma cells as described herein are particularly useful therapeutic agents according to the present invention.

In another aspect of the invention, cancers positive for surface expression of Mer are treated by administration of an agent that affects a post-translational proteolytic cleavage at the Mer extracellular domain. Such agents have been described above with respect to the treatment of clotting disorders, but can also be applied to the treatment of cancer according to the invention.

In the therapeutic methods of the invention, suitable methods of administering a composition of the present invention to a subject include any route of in vivo administration that is suitable for delivering the composition. The preferred routes of administration will be apparent to those of skill in the art, depending on the type of delivery vehicle used, the target cell population, whether the compound is a protein, nucleic acid, or other compound (e.g., a drug or an antibody) and the disease or condition experienced by the patient.

When the agent to be administered to a patient is a protein, small molecule (i.e., the products of drug design) or antibody, a preferred single dose of such a compound typically comprises between about 0.01 microgram×kilogram⁻¹ and about 10 milligram×kilogram⁻¹ body weight of an animal A more preferred single dose of an agent comprises between about 1 microgram×kilogram⁻¹ and about 10 milligram×kilogram⁻¹ body weight of an animal. An even more preferred single dose of an agent comprises between about 5 microgram×kilogram⁻¹ and about 7 milligram×kilogram⁻¹ body weight of an animal. An even more preferred single dose of an agent comprises between about 10 microgram×kilogram⁻¹ and about 5 milligram×kilogram⁻¹ body weight of an animal. Another particularly preferred single dose of an agent comprises between about 0.1 microgram×kilogram⁻¹ and about 10 microgram×kilogram⁻¹ body weight of an animal, if the agent is delivered parenterally.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention.

### EXAMPLES

### Example 1

### Human Mer Monoclonal Antibody 311 Recognizes Unique 185 kD Protein in Jurkat Leukemia Cell Line

The human Mer monoclonal antibody 311 produced by standard techniques, as described above, was tested on a Western Blot against the protein component of the human monocyte macrophage cell line U937 and the T cell leukemia line Jurkat. The human cervical carcinoma cell line HeLa was also included as a negative control. Both U937 and Jurkat express Mer mRNA by testing with RT/PCR while HeLa does not express Mer mRNA (results not shown). The 311 antibody recognized distinct size differences in the Mer protein (FIG. 1). The monocyte line U937 showed the expected banding at 205 kD. Unexpectedly, a 185 kD protein was found in the acute lymphocytic cell line (Jurkat).

FIG. 1 shows that the monoclonal antibody 311 directed against human Mer recognizes a 205 kD protein in the monocytic cell line U937 and a 185 kD protein in the Jurkat leukemia cell line on a Western blot. Hela cells, which do not express Mer mRNA by RT/PCR, were used as a negative control for this antibody.

### Example 2

### Human Mer 185 kD Protein in Leukemia Cell Lines is Distinct from Mer in Other Hematopoietic Lineages

A Western Blot of the cell line U937 (human monocyte macrophage), K562 (human myeloid leukemia), Jurkat and HSB-2 (both myeloid leukemia) and normal human platelets was probed with the 311 monoclonal antibody. The Blot was probed with the anti-Mer antibody 311. The results are shown in FIG. 2. The 311 antibody demonstrates distinctly migrating bands in all of the cell lines. Also seen in the platelet sample lane is soluble Mer extracellular domain (sMer, indicated by arrows), which is present in human plasma.

FIG. 2 is a Western blot probed with monoclonal antibody 311, demonstrating distinctly migrating forms of Mer in U937 (monocytic), K562 (myeloid), Jurkat (T-cell leukemia) and HSB-2 (T-cell leukemia) cell lines and in platelets. Also seen in the platelet sample lane is soluble Mer extracellular domain (sMer, arrows), which is present in human plasma.

### Example 3

### Deglycosylation Confirms Unique Mer Protein in Jurkat Leukemia Cells

U937, Jurkat, or K562 cell lysates were either untreated (−), digested with the glycosidase PNGase F (+PNG), or digested with a mixture of 5 glycosidases (+deglyc. mix). The 5-enzyme mix should remove all N-linked (Asn-linked) and most O-linked (Ser or Thr linked) carbohydrates from the glycoproteins. Digested lysates were fractionated by SDS-PAGE and probed with anti-Mer antibody 311 (FIG. 3). The Mer glycoform present in Jurkat leukemia cells migrates differently after each of these treatments compared to Mer from the other two cell lines, revealing differences in glycosylation patterns.

### Example 4

### Detection of Aberrant Mer Glycoforms in T Cell ALL Patient Samples

FIG. 4 illustrates a Western blot with anti-human Mer monoclonal antibody showing the control cell lines, A459 (lung carcinoma) and Jurkat (T-cell leukemia); 9 Mer positive T cell acute lymphoblastic leukemia (ALL) patient samples; 3 negative samples; and a thymus from a normal newborn. An additional 4 negative patient samples are not shown. The Blot was probed with the anit-Mer antibody 311. Two different glycosylation forms of Mer, 185 kD and 140 kD, are evident in the cell lines and patient samples. The middle panel demonstrates the presence of Axl, another member of the Mer tyrosine kinase family, only in the A459 cell line. There is no Axl detected in the T-ALL cell lines or patient samples. Actin standardization is shown in the bottom panel as a loading control.

### Example 5

### Mutations/Truncations of Mer Protein Exist in Some Patient Samples

FIGS. 5A-5C are Western blots showing the Jurkat T-ALL cell line (FIG. 5A) and two patient samples, 3877 (FIG. 5B) and 3554 (FIG. 5C), deglycosylated with the enzyme PNGase F. Mer is indicated by the arrowheads. The predominant Mer glycoform present in Jurkat cell line and patient 3877 is 185 kD before deglycosylation and 110 kD after PNGase treatment. The predominant Mer glycoform in patient 3554 is 150 kD prior to deglycosylation and 90 kD after treatment.

### Example 6

### Protein S and Gas6 are Ligands for Mer

FIG. 6 shows that Mer can be activated by Protein S or Gas6. Thymocytes from Mer transgenic mice were starved in serum free medium for 2 hours and then treated with the indicated concentrations of hProtein S or mGas6. Inhibition of Mer activation by co-incubation with Mer/Fc was also performed where indicated. Activated Mer was assessed by immunoblot blot using anti-phospho-Mer antibody.

### Example 7

### Soluble Mer is Shed into the Medium of Cultured Cells

FIGS. 7A-7D show that Mer extracellular domain is released into the medium of cultured cell lines. J774 (mouse; FIG. 7A) or U937 (human; FIG. 7B) monocytic cells were grown overnight in serum-free medium. The conditioned medium (CM) was collected and concentrated 10 fold, cells were lysed and a sample of each CM was deglycosylated with PNGase F. FIG. 7C shows the concentrated CM from overnight cultures of human cell lines. FIG. 7D shows the concentrated CM from HSB-2 and U937 cells untreated and digested with PNGase F. Cell lysates (cells), medium (CM), and PNGase-digested medium (CM+PNG) were analyzed by SDS-PAGE and immunoblotting with antibodies against mouse (FIG. 7A) or human (FIGS. 7B-7D) Mer extracellular domain.

As shown FIGS. 7A-7D, a soluble Mer protein (sMer) of 120-140 kD was shed into the medium by these macrophage cell lines. The sMer was reduced to an apparent size of 60 kD after PNGase treatment, which is consistent with the predicted size of unmodified extracellular domain cleaved close to the transmembrane sequence. Similar results indicate that the soluble protein is also release in human cells (U937).

### Example 8

### LPS and PMA Induce Mer Ectodomain Shedding

FIGS. 8A-8D show LPS or PMA stimulate release of Mer ectodomain. Surface expression of Mer on J774 cells treated with 100 ng/ml LPS for 4 hr (FIG. 8A) or treated with 50 nM PMA for 45 hr (FIG. 8B) was evaluated by flow cytometry. Expression on untreated cells at each timepoint is shown for comparison. (FIG. 8C) J774 cells were incubated in serum free medium with or without 100 ng/ml LPS or (FIG. 8D) treated with 50 nM PMA for the indicated times. Mer present in cell lysates and sMer released into the medium at each timepoint were analyzed by immunoblotting.

### Example 9

### Soluble Mer in Mice

Soluble Mer shed from peritoneal macrophages (PMΦ) and from splenocytes, as well as Mer present in normal mouse serum was analyzed (FIG. 9). Wildtype mice were injected with thioglycollate to elicit macrophage accumulation in the peritoneal cavity. Three days after injection peritoneal macrophages were collected and then cultured in serum-free medium for 7 hours. Spleens from wildtype mice were passed through a cell strainer to yield a single cell suspension, and the splenocytes were also cultured in serum-free medium for 7 hours. Cell lysates and conditioned medium from the cultured macrophages and splenocytes, and serum from wildtype mice or Mer knockout mice (KO) (negative control) were analyzed by SDS-PAGE and immunoblotting. This experiment shows that the soluble form of Mer is also released from spleen cells (in addition to cultured cells). Large amounts of soluble Mer was also detected in the serum. No proteins were detected in serum of Mer KO mice using the anti-mouse Mer antibody.

FIG. 9 shows that sMer is shed from primary cells in culture and is detected in mouse blood. Three days after wildtype C57/B6 mice were injected with thioglycollate, peritoneal macrophages were harvested and cultured in serum-free medium for 7 hours. Spleens from wildtype mice were passed through a cell strainer to yield a single cell suspension, and the splenocytes were also cultured in serum-free medium for 7 hours. Cell lysates and conditioned medium from the cultured macrophages and splenocytes, and 5 l serum from wildtype mice or mice with the Mer gene disrupted (KD) were analyzed by SDS-PAGE and immunoblotting.

### Example 10

### Soluble Mer is Present in Human Blood

Human lung microvascular endothelial cells (MVEC), human platelets and plasma samples were examined for the presence of Mer by Western blotting (FIG. 10). A 165 kD Mer protein was detected in pelleted platelets, and soluble Mer extracellular domain proteins of 110-140 kD were abundant in the plasma from this platelet preparation (labeled “platelet CM”). U937 cell lysate was a positive control for Mer receptor expression. The right panel shows platelet preparation plasma as well as two samples of pooled plasma (George King Bio-Medical, Inc.) from normal donors or from patients who had been treated with the anticoagulant coumadin. Gas6 is a vitamin K dependent ligand for Mer that are affected by coumadin. Treatment with the anticoagulant appears to increase Mer cleavage and more soluble Mer is detected.

FIG. 10 shows human lung microvascular endothelial cells (MVEC), platelets, and plasma samples were examined for the presence of Mer by Western blotting. Cultured MVEC express a 185 kD Mer glycoform, and soluble Mer was present in conditioned medium from these cells. A 165 kD Mer protein was detected in pelleted platelets, and soluble Mer extracellular domain proteins of 110-140 kD were abundant in pooled plasma from normal donors (George King Bio-Medical, Inc.) and in plasma from free-flowing blood (plasma), and subsequently after clotting, in serum from the same donor (serum).

### Example 11

### Specific Metalloprotease Inhibitor (TAPI) Blocks Production of Soluble Mer

Cultured mouse J774 cells were treated with 50 ng/ml lipopolysaccharide (LPS) to stimulate the cleavage of the Mer extracellular domain. Metalloprotease inhibitors were added to cell cultures as indicated. Mer remaining in cells and sMer released into the medium after each treatment were detected by Western blot with anti-mouse Mer (FIG. 11). As expected, the amount of soluble Mer increased in the medium after LPS treatment. Cleavage of Mer was reduced by the addition of EDTA and almost completely blocked by TAPI, a specific inhibitor of the metalloptotease TACE, which cleaves pro-TNFα and other transmembrane proteins. DMSO (which was used to dissolve TAPI) did not affect LPS-induced cleavage of Mer.

FIG. 11 shows LPS-induced production of sMer is blocked by metalloprotease inhibitors. J774 cells were treated with 50 ng/ml (LPS) to stimulate the cleavage of the Mer extracellular domain. 5 mM EDTA, 200 M TAPI-0 or DMSO (vehicle used to dissolve TAPI) were added to cell cultures as indicated for 2 hours. Mer remaining in cells and sMer released into the medium after each treatment were detected by Western blot.

### Example 12

### Mer/Fc (sMer) Binds to Gas6

The soluble Mer receptor ectodomain was shown to bind to its ligand Gas6 in this in vitro pulldown assay (FIGS. 12A-12B). A chimeric protein consisting of the Mer extracellular domain fused to the Fc region of human IgG was incubated with purified Gas6. As a control for nonspecific binding, a parallel experiment was performed with a TNFα receptor ectodomain/Fc chimera and Gas6. Complexes were pulled down with protein A Sepharose beads, run on SDS-PAGE gels, and bound Gas6 was detected by immunoblotting with anti-mouse Gas6 antibody. Receptor/Fc proteins were purchased from R&D Systems.

FIGS. 12A and 12B show that the soluble ectodomain of Mer binds to Gas6. A chimeric protein consisting of the Mer extracellular domain fused to the Fc region of human IgG was incubated with recombinant mouse Gas6. As a control for nonspecific binding, a parallel experiment was performed with a Ret receptor ectodomain/Fc chimera and mGas6. Complexes pulled down with protein G Sepharose beads and input rmGas6 were run on SDS-PAGE gels, and bound Gas6 was detected by immunoblotting with anti-mouse Gas6 antibody.

### Example 13

### Mer/Fc (sMer) Inhibits Gas6 Signaling

Soluble Mer ectodomain can block Gas6 activation of Mer in mouse cells. FIGS. 13A-13B show that the soluble ectodomain of Mer inhibits Gas6 signaling. J774 cells were starved for 2 hours in serum-free medium and then treated for 10 min. with 200 nM mGas6 and 200 nM Mer/Fc or Ret/Fc as indicated. Cell lysates were analyzed for phospho-Mer and total Mer content. As shown in FIGS. 13A and B, J774 cells incubated with or without murine Gas6 and Mer/Fc (FIG. 13A) and phospho-AKT and total AKT (FIG. 13B) was monitored.

### Example 14

### Mer/Fc (sMer) Proteins are Glycosylated Differently in Mammalian and Insect Cells

FIG. 14 shows that Mer/Fc (sMer) proteins are glycosylated differently in mammalian and insect cells. HEK293 cells were transfected with a plasmid encoding the human Mer extracellular domain coupled to the Fc region of human IgG. Shown is Mer/Fc (sMer) secreted into the culture medium by transfected HEK293 cells and a sample of Mer/Fc expressed from a baculovirus vector in Sf21 insect cells (R&D Systems).

### Example 15

### Mer Expression is Associated with Lack of Surface CD3

FIG. 15 shows that in a double-blinded retrospective chart review of 16 T cell ALL patients, there was a statistically significant association between the detection of Mer in lymphoblasts and the lack of surface expression of CD3. Lymphoblasts lacking CD3 are derived from an immature stage of thymic differentiation and have been associated with decreased event-free survival compared to CD3 surface positive lymphoblasts.

### Example 16

### Mer/Fc (sMer) Inhibits Platelet Aggregation In Vitro

FIGS. 16A-16D show that Mer extracellular domain inhibits platelet aggregation induced by ADP and collagen. In vitro platelet aggregation was performed using human platelet rich plasma and was analyzed on a BioData aggregometer. As shown in FIGS. 15A and 15B, aggregation response of platelets in response to 2 mM ADP (FIG. 16A) or 4 mM ADP (FIG. 16B) following preincubation with different concentrations of Mer/Fc. FIG. 16C shows the platelet aggregation induced by 4 mM ADP after pretreatment with Ret/Fc. FIG. 16D shows the aggregation of platelets in response to 10 g/ml collagen with and without preincubation with Mer/Fc. Squares on the X axis represent 15 second intervals. Data shown are representative of three independent experiments.

### Example 17

### Mer/Fc (sMer) Protects Against Fatal Thromboembolism

FIG. 17 shows the inactivation of the Mer gene or inhibition of Mer activation protects mice against thrombosis. Thromboembolism induced by collagen-epinephrine injection was monitored in control wildtype mice, wildtype mice pretreated with Mer/Fc protein and in mice with the Mer gene disrupted (KO).

### Example 18

### Activation of Mer with Gas6 Leads to Activation of Pro-Survival Pathways AKT and ERK ½

FIG. 18 shows that the activation of Mer with Gas6 leads to activation of pro-survival pathways AKT and ERK ½. Wildtype (WT), Mer knockout (Mer KO), and Mer transgenic (Mer Tg) thymocytes were serum starved in the serum free medium for three hours and treated with 150 nM Gas6 for 10 minutes. Cell lysates were analyzed for phospho-Mer, Mer, phospho-AKT, AKT, phospho-ERK ½, ERK ½ and actin.

### Example 19

### Mer Transgenic Mice Develop Lymphoblastic Leukemia/Lymphoma

FIG. 19 shows that Mer transgenic mice develop lymphoblastic leukemia/lymphoma. FIG. 19A shows hepatosplenomegaly in Mer transgenic mouse with lymphoblastic leukemia/lymphoma. As shown in FIGS. 19B and 19C, splenomegaly (FIG. 19B) and intra-abdominal lymphoma (FIG. 19C) are noted in Mer transgenic mice with lymphoblastic leukemia/lymphoma. FIG. 19D shows an H and E stain of intra-abdominal lymphoma confirming the presence of lymphoblasts. In FIGS. 19E and 19F, lymphoblasts from Mer transgenic mice have been characterized by flow cytometry as T cell (Thy 1.2 positive) (FIG. 19E) and Mer positive (FIG. 19F).

Each publication referenced herein is incorporated herein by reference in its entirety.

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following claims.

What is claimed is:

1. A method of diagnosing leukemia or lymphoma, comprising detecting aberrant glycoforms of Mer transmembrane receptor tyrosine kinase (Mer) in lymphocytes from an individual, wherein the expression of aberrant glycoforms of the Mer transmembrane receptor tyrosine kinase by lymphocytes in the individual is indicative of leukemia or lymphoma.

2. The method of claim 1, wherein the method comprises detecting an aberrant glycoform of Mer selected from the group consisting of: a Mer glycoform having a molecular weight between about 170 kD and about 190 kD, a Mer glycoform having a molecular weight between about 135 kD and about 140 kD, and a Mer glycoform having a molecular weight between about 190 kD and about 195 kD.

3. The method of claim 1, wherein the leukemia is a lymphoblastic leukemia, and wherein the method includes the detection of at least one Mer glycoform having a molecular weight of between about 170 kD and about 190 kD, or having a molecular weight between about 135 kD and about 140 kD.

4. The method of claim 3, wherein when a Mer glycoform having a molecular weight of between about 170 kD and about 190 kD is detected, the method further comprises detecting the amount of the Mer glycoform expressed by the cells, wherein expression of Mer and lack of CD3 expression by the cells, indicates a poor prognosis for the individual.

5. The method of claim 1, wherein the leukemia is a myelogenous leukemia or lymphoma, and wherein the method includes the detection of a Mer glycoform having a molecular weight of between about 190 kD and about 195 kD.

6. The method of anyone of claims 1 to 5, wherein the step of detection comprises contacting the sample with an antibody or antigen binding fragment thereof that selectively binds to said Mer transmembrane receptor tyrosine kinase glycoform.

7. A method of identifying a Mer-positive tumor, comprising detecting aberrant glycoforms of Mer transmembrane receptor tyrosine kinase (Mer) in lymphocytes from an individual, wherein the detecting comprises at least one technique selected from flow cytometry, Western blot analysis, and immunohistochemistry (IHC).

8. The method of claim 7, wherein the Mer-positive tumor is a solid tumor and the lymphocytes are lymphoma cells.

9. The method of claim 7, wherein the method comprises detecting an aberrant glycoform of Mer selected from the group consisting of: a Mer glycoform having a molecular weight between about 170 kD and about 190 kD, a Mer glycoform having a molecular weight between about 135 kD and about 140 kD, and a Mer glycoform having a molecular weight between about 190 kD and about 195 kD.

10. The method of claim 7, wherein the tumor is a lymphoblastic leukemia, and wherein the method includes the detection of at least one Mer glycoform having a molecular weight of between about 170 kD and about 190 kD, or having a molecular weight between about 135 kD and about 140 kD.

11. The method of claim 10, wherein when a Mer glycoform having a molecular weight of between about 170 kD and about 190 kD is detected, the method further comprises detecting the amount of the Mer glycoform expressed by the cells, wherein expression of Mer and lack of CD3 expression by the cells, indicates a poor prognosis for the individual.

12. The method of claim 7, wherein the leukemia is a myelogenous leukemia or lymphoma, and wherein the method includes the detection of a Mer glycoform having a molecular weight of between about 190 kD and about 195 kD.


SEQUENCE LISTING (8 sequences)

SEQ ID NO: 1 - DNA, 3632 bp, Homo sapiens
  CDS (123)..(3122)
actcactgcccgggccgcccggacagggagcttcgctggcgcgcttggccggcgacaggacaggttcgggacgtccatctgtccatccgtccggagagaaattacagatccgcagccccgggatggggccggccccgctgccgctgctgctgggcctcttcctccccgcgctctggcgtagagctatcactgaggcaagggaagaagccaagccttacccgctattcccgggaccttttccagggagcctgcaaactgaccacacaccgctgttatcccttcctcacgccagtgggtaccagcctgccttgatgttttcaccaacccagcctggaagaccacatacaggaaacgtagccattccccaggtgacctctgtcgaatcaaagcccctaccgcctcttgccttcaaacacacagttggacacataatactttctgaacataaaggtgtcaaatttaattgctcaatcagtgtacctaatatataccaggacaccacaatttcttggtggaaagatgggaaggaattgcttggggcacatcatgcaattacacagttttatccagatgatgaagttacagcaataatcgcttccttcagcataaccagtgtgcagcgttcagacaatgggtcgtatatctgtaagatgaaaataaacaatgaagagatcgtgtctgatcccatctacatcgaagtacaaggacttcctcactttactaagcagcctgagagcatgaatgtcaccagaaacacagccttcaacctcacctgtcaggctgtgggcccgcctgagcccgtcaacattttctgggttcaaaacagtagccgtgttaacgaacagcctgaaaaatccccctccgtgctaactgttccaggcctgacggagatggcggtcttcagttgtgaggcccacaatgacaaagggctgaccgtgtccaagggagtgcagatcaacatcaaagcaattccctccccaccaactgaagtcagcatccgtaacagcactgcacacagcattctgatctcctgggttcctggttttgatggatactccccgttcaggaattgcagcattcaggtcaaggaagctgatccgctgagtaatggctcagtcatgatttttaacacctctgccttaccacatctgtaccaaatcaagcagctgcaagccctggctaattacagcattggtgtttcctgcatgaatgaaataggctggtctgcagtgagcccttggattctagccagcacgactgaaggagccccatcagtagcacctttaaatgtcactgtgtttctgaatgaatctagtgataatgtggacatcagatggatgaagcctccgactaagcagcaggatggagaactggtgggctaccggatatcccacgtgtggcagagtgcagggatttccaaagagctcttggaggaagttggccagaatggcagccgagctcggatctctgttcaagtccacaatgctacgtgcacagtgaggattgcagccgtcaccagagggggagttgggcccttcagtgatccagtgaaaatatttatccctgcacacggttgggtagattatgccccctcttcaactccggcgcctggcaacgcagatcctgtgctcatcatctttggctgcttttgtggatttattttgattgggttgattttatacatctccttggccatcagaaaaagagtccaggagacaaagtttgggaatgcattcacagaggaggattctgaattagtggtgaattatatagcaaagaaatccttctgtcggcgagccattgaacttaccttacatagcttgggagtcagtgaggaactacaaaataaactagaagatgttgtgattgacaggaatcttctaattcttggaaaaattctgggtgaaggagagtttgggtctgtaatggaaggaaatcttaagcaggaagatgggacctctctgaaagtggcagtgaagaccatgaagttggacaactcttcacagcgggagatcgaggagtttctcagtgaggcagcgtgcatgaaagacttcagccacccaaatgtcattcgacttctaggtgtgtgtatagaaatgagctctcaaggcatcccaaagcccatggtaattttacccttcatgaaatacggggacctgcatacttacttactttattcccgattggagacaggaccaaagcatattcctctgcagacactattgaagttcatggtggatattgccctgggaatggagtatctgagcaacaggaattttcttcatcgagatttagctgctcgaaactgcatgttgcgagatgacatgactgtctgtgttgcggacttcggcctctctaagaagatttacagtggcgattattaccgccaaggccgcattgctaagatgcctgttaaatggatcgccatagaaagtcttgcagaccgagtctacacaagtaaaagtgatgtgtgggcatttggcgtgaccatgtgggaaatagctacgcggggaatgactccctatcctggggtccagaaccatgagatgtatgactatcttctccatggccacaggttgaagcagcccgaagactgcctggatgaactgtatgaaataatgtactcttgctggagaaccgatcccttagaccgccccaccttttcagtattgaggctgcagctagaaaaactcttagaaagtttgcctgacgttcggaaccaagcagacgttatttacgtcaatacacagttgctggagagctctgagggcctggcccagggctccacccttgctccactggacttgaacatcgaccctgactctataattgcctcctgcactccccgcgctgccatcagtgtggtcacagcagaagttcatgacagcaaacctcatgaaggacggtacatcctgaatgggggcagtgaggaatgggaagatctgacttctgccccctctgctgcagtcacagctgaaaagaacagtgttttaccgggggagagacttgttaggaatggggtctcctggtcccattcgagcatgctgcccttgggaagctcattgcccgatgaacttttgtttgctgacgactcctcagaaggctcagaagtcctgatgtgaggagaggtgcggggagacattccaaaaatcaagccaattcttctgctgtaggagaatccaattgtacctgatgtttttggtatttgtcttccttaccaagtgaactccatggccccaaagcaccagatgaatgttgttaagtaagctgtcattaaaaatacataatatatatttatttaaagagaaaaaatatgtgtatatcatggaaaaagacaaggatattttaataaaacattacttatttcatttcacttatcttgcatatcttaaaattaagcttcagctgctccttgatattaacatttgtacagagttgaagttgttttttcaagttcttttctttttcatgactattaaatgtaaaaatatttgtaaaatgaaatgccatatttgacttggcttctggtcttgatgtatttgataagaatgattcattcaatgtttaaagttgtataactgattaattttctgatatggcttcctaataaaatatgaataaggaagaaaaaa

SEQ ID NO: 2 - protein, 999 aa, Homo sapiens
MGPAPLPLLLGLFLPALWRRAITEAREEAKPYPLFPGPFPGSLQTDHTPLLSLPHASGYQPALMFSPTQPGRPHTGNVAIPQVTSVESKPLPPLAFKHTVGHIILSEHKGVKFNCSISVPNIYQDTTISWWKDGKELLGAHHAITQFYPDDEVTAIIASFSITSVQRSDNGSYICKMKINNEEIVSDPIYIEVQGLPHFTKQPESMNVTRNTAFNLTCQAVGPPEPVNIFWVQNSSRVNEQPEKSPSVLTVPGLTEMAVFSCEAHNDKGLTVSKGVQINIKAIPSPPTEVSIRNSTAHSILISWVPGFDGYSPFRNCSIQVKEADPLSNGSVMIFNTSALPHLYQIKQLQALANYSIGVSCMNEIGWSAVSPWILASTTEGAPSVAPLNVTVFLNESSDNVDIRWMKPPTKQQDGELVGYRISHVWQSAGISKELLEEVGQNGSRARISVQVHNATCTVRIAAVTRGGVGPFSDPVKIFIPAHGWVDYAPSSTPAPGNADPVLIIFGCFCGFILIGLILYISLAIRKRVQETKFGNAFTEEDSELVVNYIAKKSFCRRAIELTLHSLGVSEELQNKLEDVVIDRNLLILGKILGEGEFGSVMEGNLKQEDGTSLKVAVKTMKLDNSSQREIEEFLSEAACMKDFSHPNVIRLLGVCIEMSSQGIPKPMVILPFMKYGDLHTYLLYSRLETGPKHIPLQTLLKFMVDIALGMEYLSNRNFLHRDLAARNCMLRDDMTVCVADFGLSKKIYSGDYYRQGRIAKMPVKWIAIESLADRVYTSKSDVWAFGVTMWEIATRGMTPYPGVQNHEMYDYLLHGHRLKQPEDCLDELYEIMYSCWRTDPLDRPTFSVLRLQLEKLLESLPDVRNQADVIYVNTQLLESSEGLAQGSTLAPLDLNIDPDSIIASCTPRAAISVVTAEVHDSKPHEGRYILNGGSEEWEDLTSAPSAAVTAEKNSVLPGERLVRNGVSWSHSSMLPLGSSLPDELLFADDSSEGSEVLM

SEQ ID NO: 3 - DNA, 3564 bp, Mus musculus
  CDS (50)..(3034)
tggatacgtgcatctgtccggagagaactgccagatccgcggccccgcgatggttctggccccactgctactggggctgctgctgctacccgcgctctggagtggaggcactgccgagaagtgggaagagaccgagctagatcagctattttcagggcctttaccagggagactcccagtcaaccacaggccattctctgctcctcactccagccgggaccagctgccaccaccccagactggaagatcacatccagcacacacagccgctccccaggtgacctccacagcatcaaagctcctacctcctgttgcgtttaatcacaccattggacacatagtactgtcggaacataaaaatgtcaaatttaattgctccatcaatattcctaacacataccaagaaacagctggcatttcatggtggaaagatggaaaggaattgctcggggcacatcattcaatcacacagttttatcctgatgaggaaggggtatcaataattgcattgttcagcatagccagtgtgcagcgctcagacaatgggtcgtacttctgtaagatgaaggtgaacaatagagagattgtatctgatcccatatacgtggaagttcaaggactcccttactttattaagcagcctgagagtgtgaatgtcaccagaaacacagccttcaacctcacctgccaggccgtgggccctcctgagcccgtcaatatcttctgggttcaaaatagcagccgtgttaatgaaaaaccggaaaggtccccgtctgtcctaaccgtacctggtctgacagagacagcagtcttcagctgtgaggcccacaatgacaaaggactgacggtgtccaagggtgtacatatcaacatcaaagtaatcccctccccgcccactgaagtccatatcctcaacagtacagcacacagcatcctggtctcctgggtccctggttttgatggctactccccacttcagaactgcagcattcaggtcaaggaagctgaccggctgagtaatggctcagtcatggtttttaatacctctgcttcgccacatctgtatgagatccagcagctgcaagccctggctaattacagcatcgctgtgtcctgtcggaatgagattggctggtctgcagtaagcccttggattctggccagcacaacagaaggagctccatctgtagcacctttaaacatcactgtgtttctgaacgaatctaacaatatcctggatattagatggacgaagcctccaattaagcggcaggatggggaactggtgggctaccggatatctcacgtgtgggaaagcgcagggacttacaaagagctttctgaagaagtcagccagaatggcagctgggctcagattcctgtccaaatccacaatgccacctgcacagtgagaatcgcggccattactaaagggggcatcgggcccttcagtgagccagtgaatatcatcattcctgaacacagtaaggtagattacgcaccctcgtcaaccccagcccctggcaacaccgactctatgttcatcatcctcggctgcttctgtggattcattttaatcgggttaattttgtgtatttctctggccctcagaaggagagtccaggaaacaaagtttgggggagcattctctgaggaggattcccaactggtcgtaaattatagagcgaagaagtccttctgccggcgagccatcgagcttaccttgcagagcctgggagtgagcgaggagctgcagaataagctggaagatgttgtgattgacagaaaccttctggttctcggcaaagttctgggtgaaggagagtttgggtctgtaatggaaggaaatttgaagcaagaagatgggacttctcagaaggtggcagtgaagaccatgaagttggacaacttttctcaacgggagatcgaggagtttctcagcgaagcagcatgcatgaaagacttcaaccacccaaatgtcatccgacttctaggcgtgtgtatagaactgagctctcaaggcatcccgaagcccatggtgattttacccttcatgaaatacggagacctccacaccttcctgttatattcccgattaaacacaggacccaagtacattcacctgcagacactactgaagttcatgatggacattgcccagggaatggagtatctgagcaacaggaattttcttcatagggatttggcagctcgaaactgcatgttgcgggatgacatgactgtctgcgtggcagactttggcctctcaaagaagatttacagtggtgattattaccgccaaggccgcattgccaaaatgcctgtgaagtggatcgccatcgagagcctggcggaccgagtctacacaagcaaaagtgacgtgtgggcttttggcgtgaccatgtgggaaataacaacacggggaatgactccctatcccggagttcagaaccatgagatgtacgactaccttctccacggccacaggctgaagcagcctgaggactgcttggatgaactgtatgacatcatgtactcttgctggagtgctgatcccttggatcgacccaccttctctgtgttgaggctgcagctggaaaagctctccgagagtttgcctgatgcgcaggacaaagaatccatcatctacatcaatacccagttgctagagagctgcgagggcatagccaatgggccctcactcacggggctagacatgaacattgaccctgactccatcattgcctcttgcacaccaggcgctgccgtcagcgtggtcacggcagaagttcacgagaacaaccttcgtgaggaaagatacatcttgaatgggggcaatgaggaatgggaagatgtgtcctccactccttttgctgcagtcacacctgaaaaggatggtgtcttaccggaggacagactcaccaaaaatggcgtctcctggtctcaccatagtacactacccttggggagcccatcaccagatgaacttttatttgtagatgactccttggaagactctgaagttctgatgtgaagccagctgagaggaggcatgagagaaccaagcaaatacagcttcctgggatctggtggtcttagatactttgttattgctctgataaaacatcatgaccaaggcaatcttcaagagaaagtgtttaactaggtttactgtttcagggggttagagtctatgattgcagaaggaagttatgatggcaggaacagctgagtgcttatatctttaagagcaagcaggagacagagagcactctgggaaatggcacctatcttttgaaacctcaaagcctgctcccagtgacaaaagtccttcaacaggccatacctcctaatccttcccaaacaattccaccaactggagaccaaacattcaaatgtatgcgcgtattggggccattcttaagcaaaccactacactggttatacctgaggttttggtacttgttttccttaccaagtagagttcatggccggacagcaccaggtgaaagctgtcaagtcaggtttgcaaatacataaccaaggtcttgagagctcgtgccg

SEQ ID NO: 4 - protein, 994 aa, Mus musculus
MVLAPLLLGLLLLPALWSGGTAEKWEETELDQLFSGPLPGRLPVNHRPFSAPHSSRDQLPPPQTGRSHPAHTAAPQVTSTASKLLPPVAFNHTIGHIVLSEHKNVKFNCSINIPNTYQETAGISWWKDGKELLGAHHSITQFYPDEEGVSIIALFSIASVQRSDNGSYFCKMKVNNREIVSDPIYVEVQGLPYFIKQPESVNVTRNTAFNLTCQAVGPPEPVNIFWVQNSSRVNEKPERSPSVLTVPGLTETAVFSCEAHNDKGLTVSKGVHINIKVIPSPPTEVHILNSTAHSILVSWVPGFDGYSPLQNCSIQVKEADRLSNGSVMVFNTSASPHLYEIQQLQALANYSIAVSCRNEIGWSAVSPWILASTTEGAPSVAPLNITVFLNESNNILDIRWTKPPIKRQDGELVGYRISHVWESAGTYKELSEEVSQNGSWAQIPVQIHNATCTVRIAAITKGGIGPFSEPVNIIIPEHSKVDYAPSSTPAPGNTDSMFIILGCFCGFILIGLILCISLALRRRVQETKFGGAFSEEDSQLVVNYRAKKSFCRRAIELTLQSLGVSEELQNKLEDVVIDRNLLVLGKVLGEGEFGSVMEGNLKQEDGTSQKVAVKTMKLDNFSQREIEEFLSEAACMKDFNHPNVIRLLGVCIELSSQGIPKPMVILPFMKYGDLHTFLLYSRLNTGPKYIHLQTLLKFMMDIAQGMEYLSNRNFLHRDLAARNCMLRDDMTVCVADFGLSKKIYSGDYYRQGRIAKMPVKWIAIESLADRVYTSKSDVWAFGVTMWEITTRGMTPYPGVQNHEMYDYLLHGHRLKQPEDCLDELYDIMYSCWSADPLDRPTFSVLRLQLEKLSESLPDAQDKESIIYINTQLLESCEGIANGPSLTGLDMNIDPDSIIASCTPGAAVSVVTAEVHENNLREERYILNGGNEEWEDVSSTPFAAVTPEKDGVLPEDRLTKNGVSWSHHSTLPLGSPSPDELLFVDDSLEDSEVLM

SEQ ID NO: 5 - DNA, 3024 bp, Rattus norvegicus
  CDS (40)..(3024)
catctgtccggagagaactgccagatccgcggccccgcgatggttctggccccactactgctggggctgctgctgctatccgcactctggaatggaggcactgctgaaaaggaggaagaaatcaagccagatcagccattttcagggcctttaccagggagcctaccagctgaccacaggccattcttcgcccctcactccagtggggaccagctgtcaccatcccagactggaagatcacatccagcacacacagccactccccagatgacctctgcagcatcaaacctcctgcctcctgttgcatttaaaaacacaattggacgcatagtactttcggaacataaaagtgtcaaatttaattgctcgatcaacattcctaacgtgtatcaagaaacagctggcatttcgtggtggaaagatggaaaggaactgcttggggcacatcattcaatcacacagttttatcctgatgaggaaggggtatcaataattgcattgttcagcataaccagtgtgcagcgctcagacaatgggtcgtacatctgtaagatgaaggtgaacgatagagaggttgtgtctgatcccatatacgtggaagtccaaggactcccttactttactaagcagcctgagagcgtgaatgtcaccagaaacacagccttcaacctcacctgccaggctgtgggaccccctgagcccgtcaacatcttctgggttcaaaatagcagccgtgttaatgaaaatccagaaaggtccccgtctgtcctaactgtcgctggtctgacagagaccgcagtcttcagctgtgaggcccacaacgacaaaggactgacggtgtccaagggtgtacagatcaacatcaaagtcatcccttccccacccactgaagtccatatcctcaacagcacagcgcacagcatcctggtctcctgggtcccagggtttgatggctactccccacttcagaactgcagcattcaggtcaaggaagctgaccagctgagtaatggctcagtcatggtttttaatacctctgcttcgccacatctgtatgaagtccagcagctgcaagccctggctaattacagcgtcactgtgtcctgtcggaatgagattggctggtctgcagtgagcccttggatcctggccagcactacagaaggagctccagcagtggcgcctctaaacatcactgtgtttctgaacgaatccagcaacaacctggaaatcagatggacgaagcctccaattaagcggcaggatggggaactggtgggctaccggatatctcacgtgtgggagagtgcggggacttccaaagagctttctgaagaagtcagccagaatggcagctgggctcaggttcctgtccaaatgcacaatgccacctgcacagtgagaatcgccgtcatcactaaagggggcattgggcccttcagtgagccagtggacgtagccattccggagcacagtagggtagattacgcaccctcatcaaccccagcccctggcaacaccgagtctatgctcatcatcctgggctgcttctgcgggtttgttctcatggggttgattctgtatctttctctggccatcaaaaggagagtccaggaaacaaagtttgggggtgcattctccgaggaggactcccaattagttgtaaattacagagcaaagaagtccttctgtcggcgagccatcgagcttaccttgcaaagcctgggggtgagcgaggagctgcaaaacaagctagaagatgttgtggttgacagaaatcttctaattcttgggaaagttctgggcgaaggagagtttgggtctgtaatggaaggaaatctgaagcaggaagatgggacttctcagaaggtggcagtgaagaccatgaagttggacaacttttctctacgggagatcgaggagtttctcagcgaagcagcgtgcatgaaagacttcaatcacccaaacgtcatccggcttctaggcgtgtgtatagaactgagctctcaaggcatcccgaagcccatggtgattttacccttcatgaaatacggagacctccacaccttcctgctatattcccggatagaatcagtaccgaagtccatccccctgcagacactgctgaagttcatggtggacattgcccagggaatggagtacctgagcagcaggaattttctccacagggatttagctgctcggaattgcatgttgcgggatgacatgactgtctgcgtggcagactttggcctctctaagaagatttacagtggtgattattaccgccaaggccgcattgccaaaatgcctgtgaagtggatcgccatagagagcctggcggaccgagtctacacaagcaagagtgacgtgtgggcttttggcgtgaccatgtgggaaatagcaacacggggaatgactccctatcctggagtccagaaccatgagatgtacgattaccttctccacggccacaggctgaagcagcccgaggactgcctggatgatctgtatgaaatcatgtactcttgctggagtgctgatcccctggaccgacccaccttctcagtgctgaggctgcagctggaaaagctctccgagagtttgcctgatgcccaggacaaagaatccatcatctacatcaatacacagttactagagagctgcgagggcctagccaacaggtcctccctcgcagggctagacatgaacattgatcctgactccatcattgcctcttgcacagcaggtgctgctgtcagcgtggtcatggcggaagttcacgagaacaaccttcatgaggaaagatacatcttaaatgggggcaacgaggaatgggaagatgtggcctccactccatttgctacagtcacagctggaaaagatggtgtgttaccagaggacagactcaccaaaaatggcatctcctggtctcaccatagtacgctacccttggggagcccatcaccagatgaacttctgtttgcagacgactcctcgggagactctgaagttctgatgtga

SEQ ID NO: 6 - protein, 994 aa, Rattus norvegicus
MVLAPLLLGLLLLSALWNGGTAEKEEEIKPDQPFSGPLPGSLPADHRPFFAPHSSGDQLSPSQTGRSHPAHTATPQMTSAASNLLPPVAFKNTIGRIVLSEHKSVKFNCSINIPNVYQETAGISWWKDGKELLGAHHSITQFYPDEEGVSIIALFSITSVQRSDNGSYICKMKVNDREVVSDPIYVEVQGLPYFTKQPESVNVTRNTAFNLTCQAVGPPEPVNIFWVQNSSRVNENPERSPSVLTVAGLTETAVFSCEAHNDKGLTVSKGVQINIKVIPSPPTEVHILNSTAHSILVSWVPGFDGYSPLQNCSIQVKEADQLSNGSVMVFNTSASPHLYEVQQLQALANYSVTVSCRNEIGWSAVSPWILASTTEGAPAVAPLNITVFLNESSNNLEIRWTKPPIKRQDGELVGYRISHVWESAGTSKELSEEVSQNGSWAQVPVQMHNATCTVRIAVITKGGIGPFSEPVDVAIPEHSRVDYAPSSTPAPGNTESMLIILGCFCGFVLMGLILYLSLAIKRRVQETKFGGAFSEEDSQLVVNYRAKKSFCRRAIELTLQSLGVSEELQNKLEDVVVDRNLLILGKVLGEGEFGSVMEGNLKQEDGTSQKVAVKTMKLDNFSLREIEEFLSEAACMKDFNHPNVIRLLGVCIELSSQGIPKPMVILPFMKYGDLHTFLLYSRIESVPKSIPLQTLLKFMVDIAQGMEYLSSRNFLHRDLAARNCMLRDDMTVCVADFGLSKKIYSGDYYRQGRIAKMPVKWIAIESLADRVYTSKSDVWAFGVTMWEIATRGMTPYPGVQNHEMYDYLLHGHRLKQPEDCLDDLYEIMYSCWSADPLDRPTFSVLRLQLEKLSESLPDAQDKESIIYINTQLLESCEGLANRSSLAGLDMNIDPDSIIASCTAGAAVSVVMAEVHENNLHEERYILNGGNEEWEDVASTPFATVTAGKDGVLPEDRLTKNGISWSHHSTLPLGSPSPDELLFADDSSGDSEVLM

SEQ ID NO: 7 - DNA, 3037 bp, Gallus gallus
  CDS (26)..(2950)
gaattccgctcggtgcgggcacgggatgggcggcgggcgctgcgcgctgctctgcgcgttgctctgcgctctgcccctcccgcgctgcggggccgctgagtttggtggtgctggtggacatggaggcctgaggtccctatctgcaggagccccgctggcaaggcccctgtgggcaaagcaccaccgccccaaacgaggcctcaccagtggccgctggcctccccagggcgcaaagccttctgccacctccgtgggacagctgaaatttaaccccacagtgggacacgttgtgataaatgagctcaaagatgtcacatttaactgctccatcaaagtacctcagctgctagtccggccagactcccctggcatttccctgtggaaggatggcagggagctgcacacgctggaccgcatcgccaccagccactttgagatccttgatgaggaggaggtagccatgacctctacattcagcatccgtgctgctcagcgctcggataacggctcctacgtctgcaaactcaacatctctggcattgagattgcatctgatcccatcttggtacagctggaagggctcccacacttcattcaacagcctgagaagctgaatgtcaccaggaacagccccttcaacctcacgtgccaagctgtgggcccaccagagcctgtggaaatctactggtttcgtaacaatgtccaactcaacatgaagccctacatctccccatcagttctgactgtcccaggtctcaatgaaacagcactgttcagttgtgaggctcacaacagcaaagggctgactgcttccaaccccgggcaggtcaacgtgaaaggaataccatctgcaccaaaagctgtgcatgtcctgaagagaatggcccacagcattgtgatctcctgggtgccaggcttcgatgcgttctctgccttgaacagctgcagtgtgcaggtcaaggaagccgttccacaaagcaatgtctcacttctgctctttaacacgtcggtgcctccccatgtgtatcgcatccagcagctgtggcccatggcagactataacatcagtgtttcctgcaagaatgaagtcggttggtcggcatttagcccctggataacagccagtaccacggaaggagctccaactacccagccactgaatgtcacagtgtcactcaacgaatccagctccttcctggaaatccgatgggtgaagccaccccttgagaggacacacggggagctgcagggatatcatatctggcacacgtggcaggactccaaggggctgcagaacatctccttggaagcccagcctaatgccacagtggccatcctgcctgtggtggccaccaatgccacgtgctcagtgcgtgtggctgctgtcaccaagggaggcgtggggcccttcagcagcccagtggaggtctttgttcctgccagtgggctaataacctcatctccctcttcgacaccagcatctgggaacacagactcctttatagtagcactgggcttcgtctgtggtacggttgctgttgggctgatcctctgcttgtctgtggtcatccagaaaagatgcatggaaacaaagtatgggaatgccttcagcagaaatgattcagagctggtggtaaactacacagccaagaagtcctactgccggagagccgtcgaactgacattgggtagcctgggagtcagcagcgagctccagcagaagctgcaggacgttgtcattgacagaaatgccctcagcctggggaaggtcctgggagagggggagttcgggtcagtgatggagggacgtctcagccagccagaaggcaccccacagaaggtggctgtcaagaccatgaagttggataacttttcccatagagagatagaagaattcctcagtgaagcagcatgcatgaaggactttgaccaccccaatgtcatcaagctcctaggtgtgtgcatcgagctgagctctcagcagatccccaagcccatggtggttctcccattcatgaaatatggtgacctgcacagcttcctgcttcgctcccggctggagatggccccccagttcgtgcccctgcagatgctgctgaagttcatggtggatattgccctgggaatggagtacctgagcagtcggcagtttcttcacagggatttggcggctcggaactgcatgttacgggatgacatgacggtgtgtgtggcagactttgggctgtccaagaagatctacagcggcgattactaccgtcagggccgaatagcaaaaatgccagtgaagtggattgcgatagagtccctggctgaccgtgtctacaccaccaagagtgatgtgtgggcatttggcgttaccatgtgggagatagcgaccagagggatgactccgtacccaggggtgcagaaccacgagatttatgagtatctattccacgggcagcggctcaaaaagcctgagaactgcttagatgaactgtacgatatcatgtcctcctgctggagggctgaacctgctgaccgaccgacgttctcacagctgaaagttcatctggagaagcttttggaaagccttcctgccccgagggggtccaaggacgtcatctacgtcaacaccagcctgccagaggagagccccgactccacccaggatttgggcttggattcggtcatcccccaagcggactctgacttggaccccggggacattgctgagccctgctgctcccacacgaaggcagcgctggtggcagtggatatccacgatgggggctcgaggtacgtccttgaaagtgagggcagccccacagaggatgcttacgtcccactgctgccccacgagggctcggcgtggaccgaggccagcaccttgcctgttggcagctcgcttgcagctcagctgccatgtgctgatggctgcctggaggactccgaagcgctgctgtgaatggagcctgcgcaaagcagggtgtgaatcaggacagagagattttattttaataaacagtcttatttcttcttgcattattttcat

SEQ ID NO: 8 - protein, 974 aa, Gallus gallus
MGGGRCALLCALLCALPLPRCGAAEFGGAGGHGGLRSLSAGAPLARPLWAKHHRPKRGLTSGRWPPQGAKPSATSVGQLKFNPTVGHVVINELKDVTFNCSIKVPQLLVRPDSPGISLWKDGRELHTLDRIATSHFEILDEEEVAMTSTFSIRAAQRSDNGSYVCKLNISGIEIASDPILVQLEGLPHFIQQPEKLNVTRNSPFNLTCQAVGPPEPVEIYWFRNNVQLNMKPYISPSVLTVPGLNETALFSCEAHNSKGLTASNPGQVNVKGIPSAPKAVHVLKRMAHSIVISWVPGFDAFSALNSCSVQVKEAVPQSNVSLLLFNTSVPPHVYRIQQLWPMADYNISVSCKNEVGWSAFSPWITASTTEGAPTTQPLNVTVSLNESSSFLEIRWVKPPLERTHGELQGYHIWHTWQDSKGLQNISLEAQPNATVAILPVVATNATCSVRVAAVTKGGVGPFSSPVEVFVPASGLITSSPSSTPASGNTDSFIVALGFVCGTVAVGLILCLSVVIQKRCMETKYGNAFSRNDSELVVNYTAKKSYCRRAVELTLGSLGVSSELQQKLQDVVIDRNALSLGKVLGEGEFGSVMEGRLSQPEGTPQKVAVKTMKLDNFSHREIEEFLSEAACMKDFDHPNVIKLLGVCIELSSQQIPKPMVVLPFMKYGDLHSFLLRSRLEMAPQFVPLQMLLKFMVDIALGMEYLSSRQFLHRDLAARNCMLRDDMTVCVADFGLSKKIYSGDYYRQGRIAKMPVKWIAIESLADRVYTTKSDVWAFGVTMWEIATRGMTPYPGVQNHEIYEYLFHGQRLKKPENCLDELYDIMSSCWRAEPADRPTFSQLKVHLEKLLESLPAPRGSKDVIYVNTSLPEESPDSTQDLGLDSVIPQADSDLDPGDIAEPCCSHTKAALVAVDIHDGGSRYVLESEGSPTEDAYVPLLPHEGSAWTEASTLPVGSSLAAQLPCADGCLEDSEALL